(19) United States
(12) Patent Application Publication
(10) Pub. No.: US 2023/0131596 A1
(43) Pub. Date: Apr. 27, 2023

(54) CUSTOMIZABLE COMMUNICATION PLATFORM BUILDING

(71) Applicant: CAREMINDR Corporation, Los Gatos, CA (US)

(72) Inventors: Harry Raymond Soza, San Jose, CA (US); Irina Yermilov, Los Angeles, CA (US)

(73) Assignee: CAREMINDR Corporation, Los Gatos, CA (US)

(21) Appl. No.: 17/879,943

(22) Filed: Aug. 3, 2022

Related U.S. Application Data
(60) Provisional application No. 63/272,016, filed on Oct. 26, 2021.

(51) Int. Cl. G16H 40/20; G06F 16/22; H04L 67/02

(52) U.S. Cl. CPC G16H 40/20; G06F 16/2282; H04L 67/02

(57) ABSTRACT

A method of building a treatment plan method may include receiving a set of queries of a health related issue, wherein the response is at least one of biometric, objective and subjective, parsing the set of queries to determine keywords and key questions, searching a healthcare provider database to find alert guidelines for the keywords and key questions, populating a programming list with the set of queries and the alert guidelines for those queries and creating a query list based on the programming list.

### TECHNICAL FIELD OF THE APPLICATION

This application relates to a customizable communication platform and more particularly to providing customized healthcare communication to a user device by integrating various personal records with an ongoing communication regiment, wherein the communication may include tags pertaining to urgent healthcare issues.

### BACKGROUND OF THE APPLICATION

Conventionally, the approach to providing users with ongoing communications regarding a multi-step healthcare plan or other repetitive course of action may leave the majority of the work to the user. In this case, smartphones and other personal computing devices may not be optimally utilized by a professional service provider when offering users options for maintaining a course of treatment or a set of goals. As a result, the professional service provider may not effectively communicate with the user and the user may not adequately understand the next steps. This lack of effectiveness can lead to health problems for the user and lost work and revenue for providers, insurers, etc., as well as the users.

### BRIEF DESCRIPTION OF THE DRAWINGS

FIG. 1A illustrates an example of the integrated application platform according to example embodiments.

FIG. 1B illustrates an example of communication between a software stack of the front-end and back-end of the software application according to example embodiments.

FIG. 1C illustrates an example of an HTTP request message that may be transmitted between the front-end and back-end of the software application according to example embodiments.

FIG. 1D illustrates an example of a process of tagging patient status bubbles with visual alerts according to example embodiments.

FIG. 1E illustrates an example of a tag-to-alert mapping table according to example embodiments.

FIG. 2 illustrates a network configuration of the third-party participants of the integrated application according to example embodiments.

FIG. 3 illustrates a user smartphone interface of an example treatment plan according to example embodiments.

FIG. 4A illustrates an example setup configuration for a new course of treatment according to example embodiments.

FIG. 4B illustrates an example database entry for the new course of treatment according to example embodiments.

FIG. 4C illustrates a flow diagram configuration for the new course of treatment according to example embodiments.

FIG. 4D illustrates an example list of messages for the ongoing course of treatment according to example embodiments.

FIG. 4E illustrates an example setup configuration for various courses of treatment according to example embodiments.

FIG. 4F illustrates an example set of details of an ongoing course of treatment according to example embodiments.

FIG. 4G illustrates an example network configuration of the various third parties involved in the application operation and compliance according to example embodiments.

FIG. 5 illustrates a logic module configured to process the input and output parameters of the application according to example embodiments.

FIG. 6 illustrates an example network entity device configured to store instructions, software, and corresponding hardware for executing the same, according to example embodiments of the present application.

FIG. 7A illustrates a first portion of a data file defining tags and levels of urgency, according to example embodiments of the present application.

FIG. 7B illustrates a second portion of the data file defining tags and levels of urgency, according to example embodiments of the present application.

FIG. 8 illustrates a patient reply, according to example embodiments of the present application.

FIG. 9A illustrates a first portion of an electronic report to a healthcare provider with alert tags, according to example embodiments of the present application.

FIG. 9B illustrates a second portion of the electronic report to a healthcare provider with alert tags, according to example embodiments of the present application.

FIG. 10 illustrates system architecture, according to example embodiments of the present application.

FIG. 11 illustrates a first method, according to example embodiments of the present application.

FIG. 12 illustrates a second method, according to example embodiments of the present application.

FIG. 13 illustrates a first non-transitory computer readable medium, according to example embodiments of the present application.

FIG. 14 illustrates an example onboarding session with a patient, according to example embodiments of the present application.

FIG. 15 illustrates example questions asked of a patient, according to example embodiments of the present application.

FIG. 16 illustrates an example of the layout of the questions asked by the system, according to example embodiments of the present application.

FIG. 17 illustrates an example of the files used to program the system are placed, according to example embodiments of the present application.

FIGS. 18A-18B are diagrams illustrating an Excel file with some information for the hypertension and weekly blood pressure journey, according to example embodiments of the present application.

FIG. 19 illustrates another example Excel file with some information for the hypertension and weekly blood pressure journey, according to example embodiments of the present application.

FIG. 20 illustrates an example of message texts associated with the hypertension and weekly blood pressure journey, according to example embodiments of the present application.

FIG. 21 illustrates a portion of the Excel form and indicates answers to questions, according to example embodiments of the present application.

FIG. 22 illustrates an example of guardrail values and their possible resetting by medical personnel, according to example embodiments of the present application.

FIG. 23 illustrates a review chart having the most critical patients sorted by criticality, according to example embodiments of the present application.

FIG. 24 illustrates a review of the topmost critical patient, according to example embodiments of the present application.

FIG. 25 illustrates environmental condition questions that may have an impact on health issues, according to example embodiments of the present application.

FIG. 26 illustrates example questions related to spreading illnesses such as COVID-19, according to example embodiments of the present application.

FIG. 27 illustrates example questions related to spreading illnesses such as COVID-19, according to example embodiments of the present application.

FIG. 28 illustrates an example of a hypertension and weekly blood pressure journey message flow for different blood pressures, according to example embodiments of the present application.

FIGS. 29A-29J are diagrams illustrating a foundation journey according to example embodiments of the present application.

FIGS. 30A-30H are diagrams illustrating a journey for all medications according to example embodiments of the present application.

FIGS. 31A-31J are diagrams illustrating a pediatric asthma journey according to example embodiments of the present application.

FIGS. 32A-32F are diagrams illustrating a depression journey according to example embodiments of the present application.

FIGS. 33A-33E are diagrams illustrating a diabetes journey according to example embodiments of the present application.

FIGS. 34A-34E are diagrams illustrating a hypertension journey according to example embodiments of the present application.

FIGS. 35A-35D are diagrams illustrating a workplace monitoring journey according to example embodiments of the present application.

FIG. 36 illustrates a method of displaying an alert via a software application.

### DETAILED DESCRIPTION OF THE APPLICATION

It will be readily understood that the components of the present application, as generally described and illustrated in the figures herein, may be arranged and designed in a wide variety of different configurations. Thus, the following detailed description of the embodiments of a method, apparatus, and system, as represented in the attached figures, is not intended to limit the scope of the application as claimed, but is merely representative of selected embodiments of the application.

The features, structures, or characteristics of the application described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present application. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

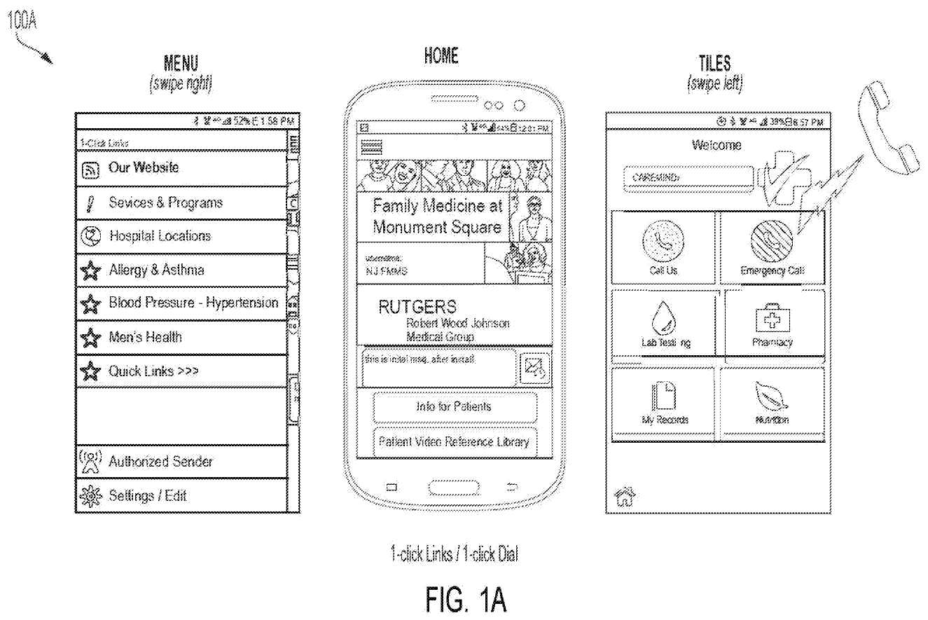

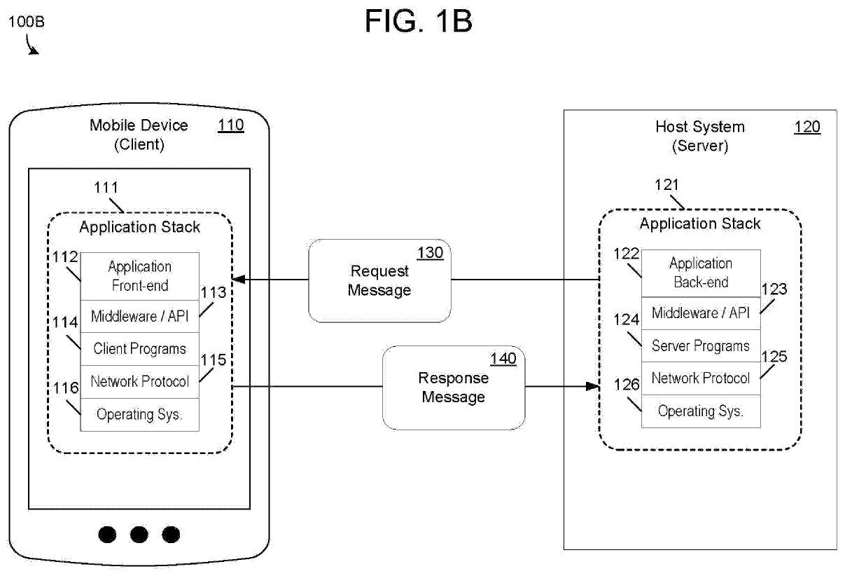

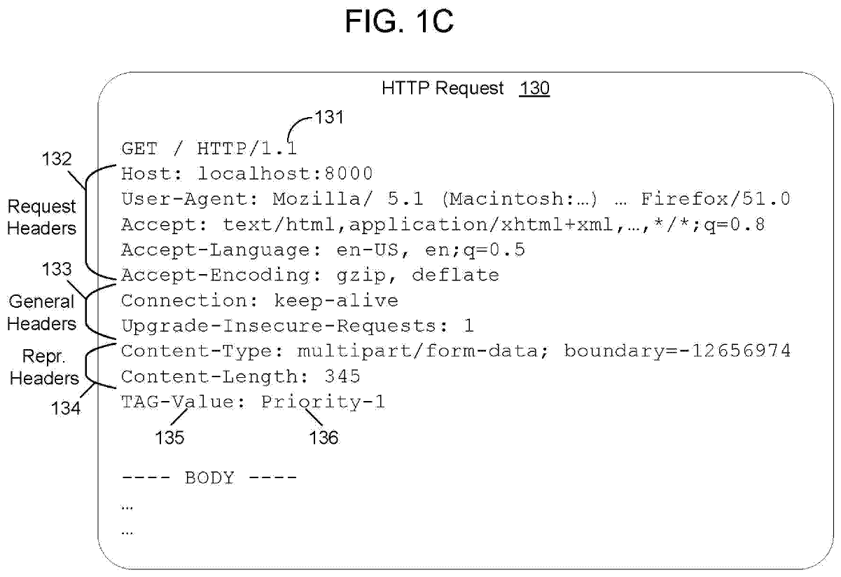

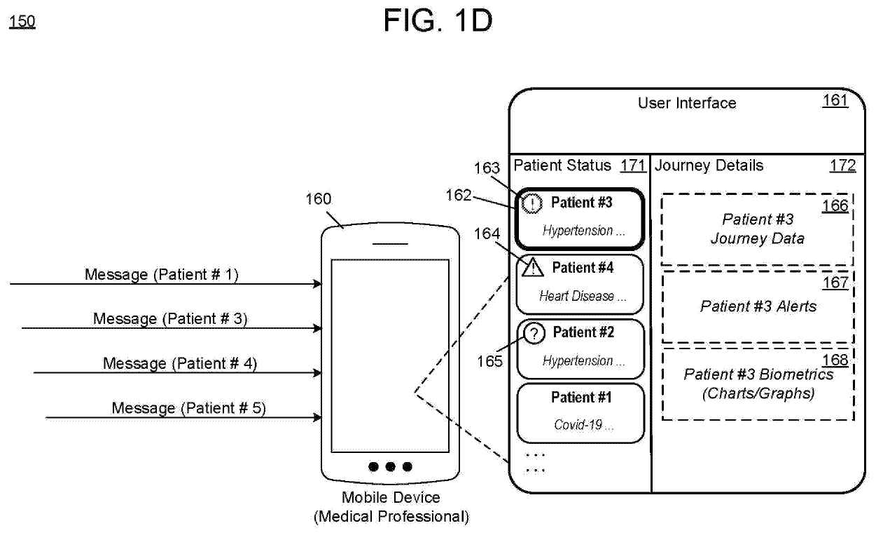

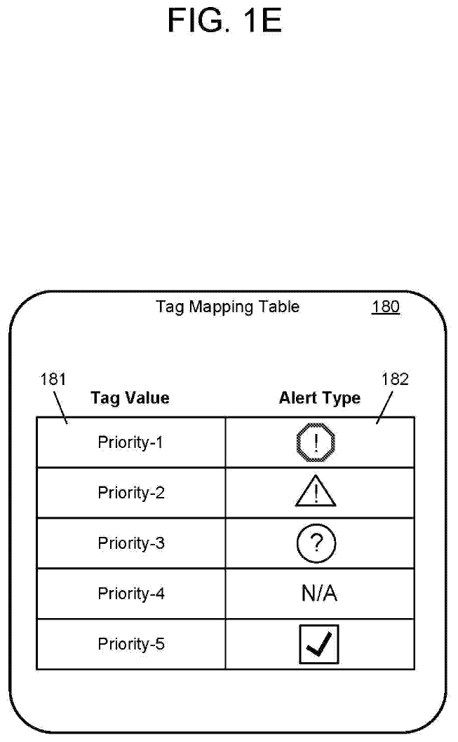

FIG. 1A illustrates an example of the integrated application platform 100 according to example embodiments. Referring to FIG. 1A, the application platform 100 includes a user interface that may be interacted with by a user based on touch commands such as swipe commands or other user interface interactions. In this example, a home user interface 102 is initially displayed in the middle of the page when the application is first launched on a mobile device of a user such as a patient being remotely monitored by the application. The user interface 102 may include details of the patient and the patient's journey, etc. If the user (e.g., the patient, etc.) were to swipe right on the home user interface 102, the application may open or otherwise navigate to a menu user interface 104 with links to additional pages and content of the application that can be accessed with a single click or other user interface actions. As another example, if the user were to swipe left on the home user interface 102, the application may open a tiles menu 106 with selectable tiles for accessing third-party resources such as emergency services, educational materials, the application technical assistance contact, lab test results, medical records, pharmacy information, and the like.

FIG. 1B illustrates an example of communication process 100B between a front-end software stack 111 and a back-end software stack 121 of the software application according to example embodiments. Referring to FIG. 1B, the software application described herein may be a mobile application, a progressive web application, a browser-based web application, and/or the like, which includes a front-end or client-side and a back-end or server-side. In FIG. 1B, a host system 120 (e.g., host of the software application described herein, etc.) performs the role of the server. The host system 120 may include one or more of a could platform, a web server, a virtual machine, a database, a distributed network of computers, and the like. The host system 120 may register users/clients such as medical professionals and patients.

Meanwhile, the patients and the medical professionals may perform the role of the client-side and may access and install the front-end application stack 111 via a mobile device 110 such as a tablet, a smartphone, a laptop, a notebook, a medical machine, or the like. For example, a medical professional may use mobile device 110 to download and install the front-end application stack 111 of the application. The user may also use the front-end application stack 111, such as programs, user interfaces, processes, functions, etc. within an application front-end 112 to register with the host system 120 and setup one or more patients of the medical professional. As another example, a patient could use mobile device 110 to register with the host system 120 as a patient of one or more medical professionals that are also participating.

As noted in FIG. 1B, the front-end application stack 111 and the back-end application stack 121 include common components, including middleware 113 and 123, respectively, network protocol configurations 115 and 125, respectively, and operating system configurations 116 and 126. Meanwhile, the front-end application stack 111 may include an application front-end 112 while the back-end application stack 121 may include an application back-end 122. The application back-end 122 may include server-side programs, functions, content, and the like, which are not shared with an application front-end 112. Likewise, the application front-end 112 may include other programs, functions, user interface, content, and the like, which are not included in the application back-end 122.

The middleware 113 and 123 is the software which connects the application front-end 112 and the application back-end 122. It enables connectivity between the client and the server and can enhance process management, user engagement, authentication, content management, and the like. The middleware 113/123 may also include one or more application programming interfaces (APIs) which allow the client-side or the front-end application 112 on the mobile device 110 to access (e.g., read, write, modify, create, delete, etc.) data stored by the host system 120 (or other third-parties), software functions, and other software and data resources of the back-end application 122 on the host system 120. In addition, the application-front end 112 may access client-specific programs and content 114 such as executable scripts, style sheets (e.g., CSS, etc.), and the like, when displaying user interfaces, populating messages, retrieving user preferences, and the like. Meanwhile, the application back-end may access server-specific programs and content 124 such as executable scripts, style sheets (e.g., CSS, etc.), and the like, which are held/contained by the server-side and not the client-side.

In the example of FIG. 1B, the host system 120 sends a request message 130 to the mobile device 110. In particular, the back-end application 122 sends the request message 130 to the front-end application 112, via network configurations and other information contained in the software stack 121. Likewise, the application front-end 112 may transmit a response message 140 to the back-end application 122 on the host system 120.

FIG. 1C illustrates an example of a Hypertext Transfer Protocol (HTTP) request message 130 that may be transmitted in FIG. 1C, however, embodiments are not limited thereto. It should also be appreciated that the messages may not be in HTTP form, but may be in another format. The messages may include instant messages that are transmitted in-app. As another example, the messages may also include SMS messages, emails, or other types of electronic messages.

Referring to FIG. 1C, the request message 130 includes a start line 131 describing the requests to be implemented including a type of command (e.g., POST, PUT, GET, etc.) and a URL/network address. For example, “GET” refers to data being fetched by the server while “POST” refers to data being pushed to the server. The request message 130 also includes optional headers such as request headers 132, general headers 133, representative headers 134, and the like.

According to various embodiments, the messages that are transmitted between the front-end and the back-end of the application described herein may include tag values (e.g., tag value 136 in tag field 135. The tag values may represent visual tags that are to be displayed on a user interface, such as a patient status screen. The tags may represent priority among the different patients such as priority based on urgency of care needed. The host system may determine the urgency based on the response from a patient or lack of responses from the patient, and may generate an alert of some kind in response. The alert may be a visual alert that is to be displayed on a screen of a medical professional.

FIG. 1D illustrates an example of a process 150 of tagging patient status bubbles (e.g., bubble 162, etc.) with visual alerts (e.g., visual alert 163, etc.) according to example embodiments. Referring to FIG. 1D, the mobile device 160 may receive messages from the host system (not shown) with updates to patient status. In response, the application may generate a user interface 161, or update the user interfaced 161, with details from the received messages. In the example of FIG. 1D, the user interface 160 includes a patient status window 171 and a journey details window 172 within the user interface 161 which display additional details from the messages.

The patient status window 171 includes a plurality of message bubbles such as bubble 162 with status information of Patient #3. In this example, the sender of the messages (e.g., the host system 120 of the back-end software application 122 in FIG. 1B, etc.) may insert a respective alert tag into each message indicating a level of urgency of each respective patient. In response, the mobile device 160 (e.g., the front-end of the application 112 in FIG. 1B, etc.) may read the alert tag and generate a visual alert icon, graphic, or other visual element on the screen.

In FIG. 1D, the host system embeds a tag that identifies Patient #3 as having the most severe urgency level. In response, the mobile device may populate the bubble 162 of Patient #3 with the visual alert 163. The visual alert 163 may be embedded with the status bubble of Patient #3 thereby enabling the viewer to easily and quickly comprehend that Patient #3 needs attention first. In addition, the front-end of the application may arrange, order, re-arrange, etc., the ordering of the bubbles in the patient status window 171 based on urgency. For example, the application may move status bubbles of patients with the most severe level of urgency to the top, followed by patients with a less severe level of urgency, etc. Also, the application may populate the journey details window 172 with patient data of the currently selected patient within the patient status window 171. The patient data may include journey data 166, alert messages 167, biometrics 168 such as charts, graphs, readings, etc., and the like. In this example, the currently selected patient is Patient #3 because the user pressed/touched the message bubble 162 of Patient #3.

FIG. 1E illustrates an example of a tag-to-alert mapping table 180 according to example embodiments. Referring to FIG. 1E, the table 180 includes a plurality of tag values stored in a first column 181 that are paired with (i.e., mapped to) a plurality of alert types in a second column 182. The plurality of tag values may include a plurality of identifiers that can be embedded within tag fields of messages transmitted between the front-end and back-end of the application, and vice versa. Meanwhile, the alert types include visual alerts (e.g., icons, graphics, symbols, images, etc.) which should be displayed on the screen in response to receiving a message with a corresponding tag value. In some embodiments, the alert types may include attributes of the visual display such as CSS styling information, one or more scripts for executing and displaying the alert type and additional data, and the like.

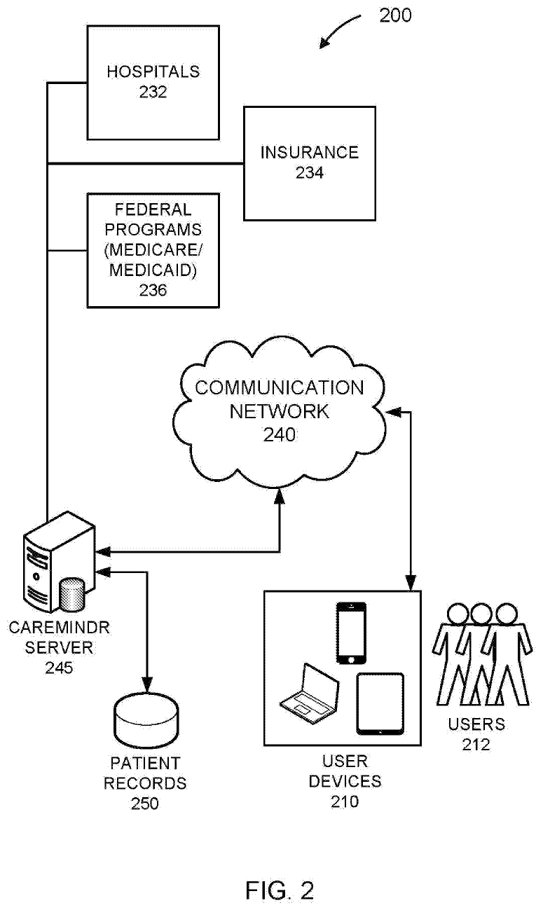

FIG. 2 illustrates a network configuration of the third-party participants of the integrated application according to example embodiments. Referring to FIG. 2, the network includes a central server 245 with patient records 250. The information needed to provide treatment plans and other integrated services may require access to hospital and other provider services 232, insurance company information 234, drug providers, federal program administrators 236, etc. The information may be incorporated into any treatment plan or other integrated service model accessed by a user device 210 operated by a user 212. The servers and third-party modules may operate on-site or in a cloud network managed by the providers.

Examples of treatment plans and other objectives may include a care management service for assessment of patient medical needs. The system and application may ensure timely receipt of all recommended treatment actions, drugs, third-party services and over a designated period. Also, referrals to other providers and additional services may provide emergency visits, discharge instructions, nursing facility operations, and home health care functions. In operation, the procedure may begin with the medical treatment provider creating a treatment plan or ‘journey’ for each patient. Each journey is generally for a single chronic condition or objective. One patient may have multiple journeys integrated into a single application. Also, the journeys may originate from various providers and service entities. The journey will provide the healthcare provider with biometric, objective and subjective data to enable evidence-based medical decisions. As an example, the biometric data may be glucometer data collected from a blue tooth enabled device and made available to the physician, objective data such as whether the patient visited an emergency room or hospital and subjective data such as how the patient is feeling.

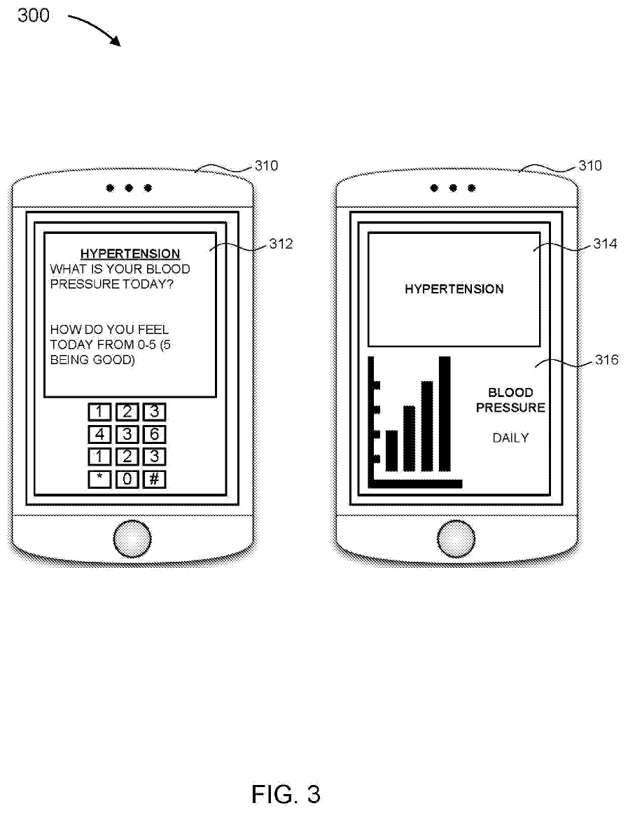

FIG. 3 illustrates a user smartphone interface of an example treatment plan according to example embodiments. Referring to FIG. 3, the journey for “hypertension” may have been created or modified by a patient doctor and may include an interface 300 with a smartphone device 310 and a screen option configuration providing questions 312, information about the treatment, reminders and other functions. The example in FIG. 3 provides for a set of questions 312 and a journey topic 314 along with a graph of blood pressure records 316 as measured over time from various interactions.

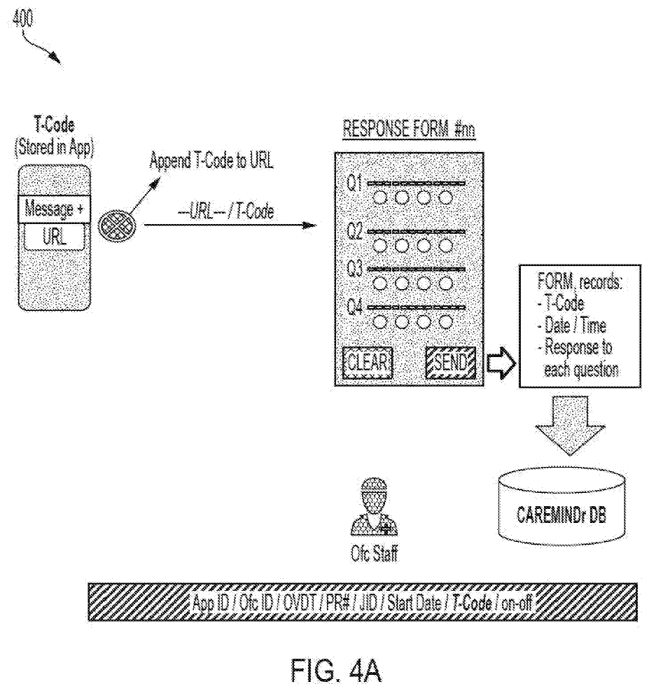

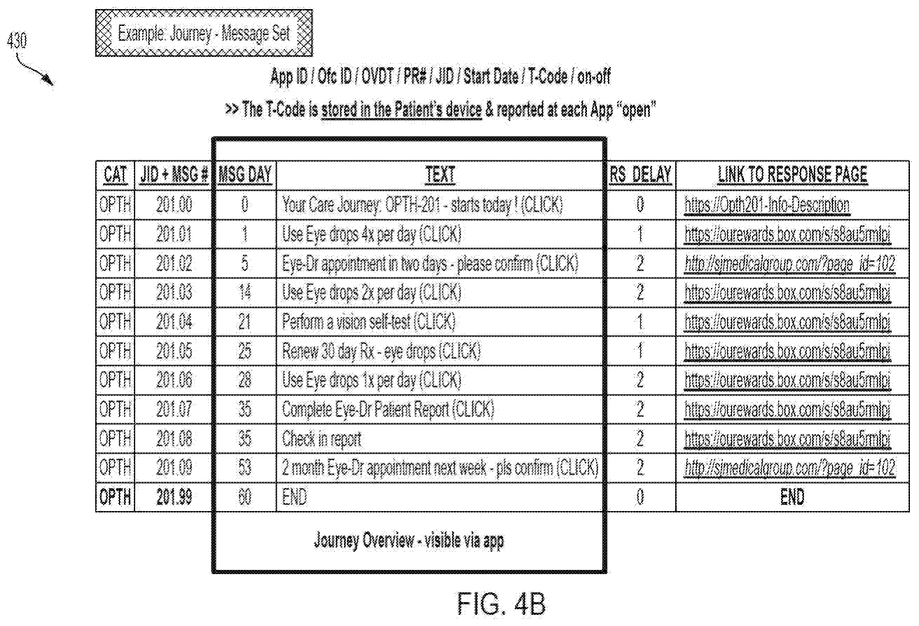

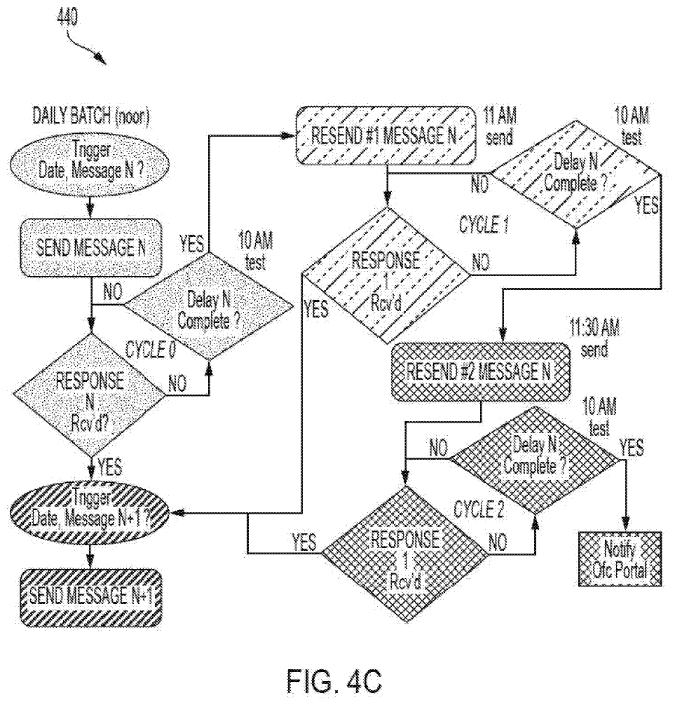

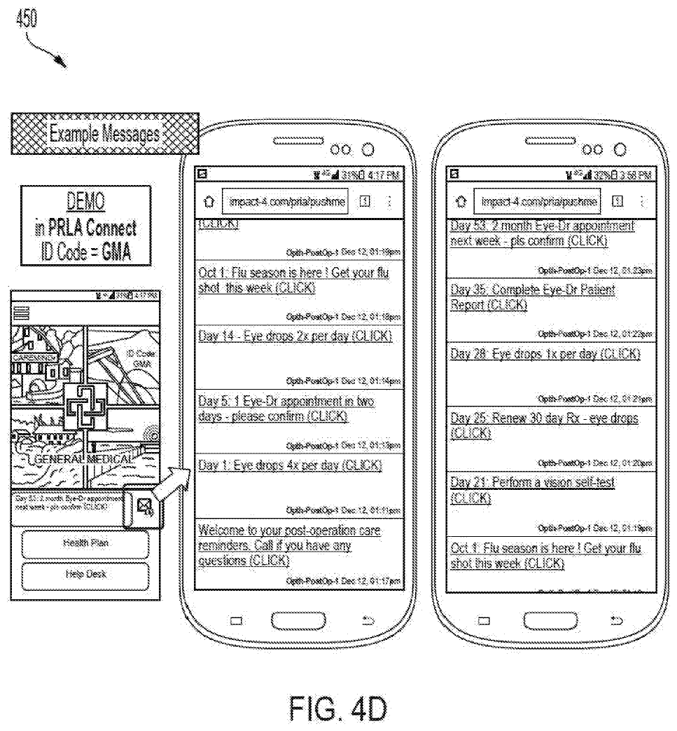

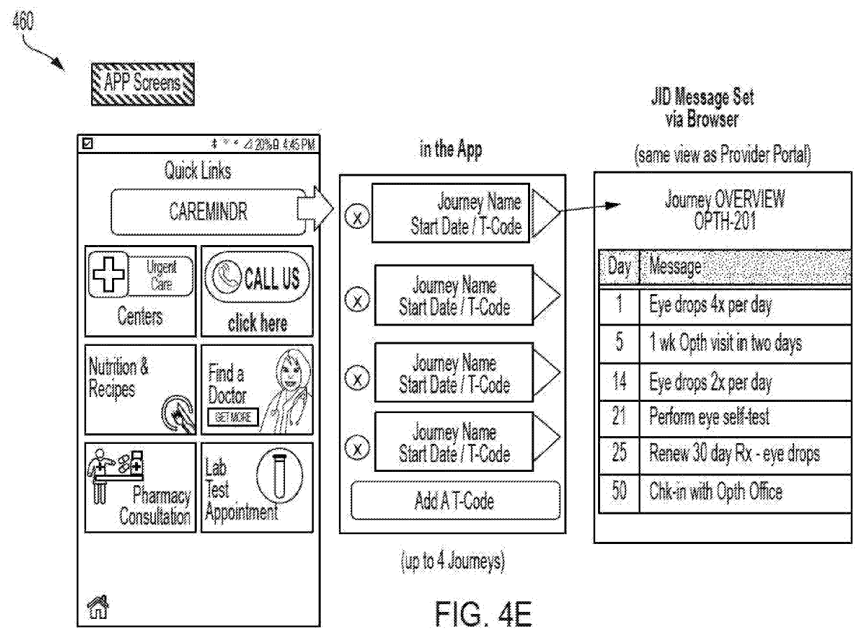

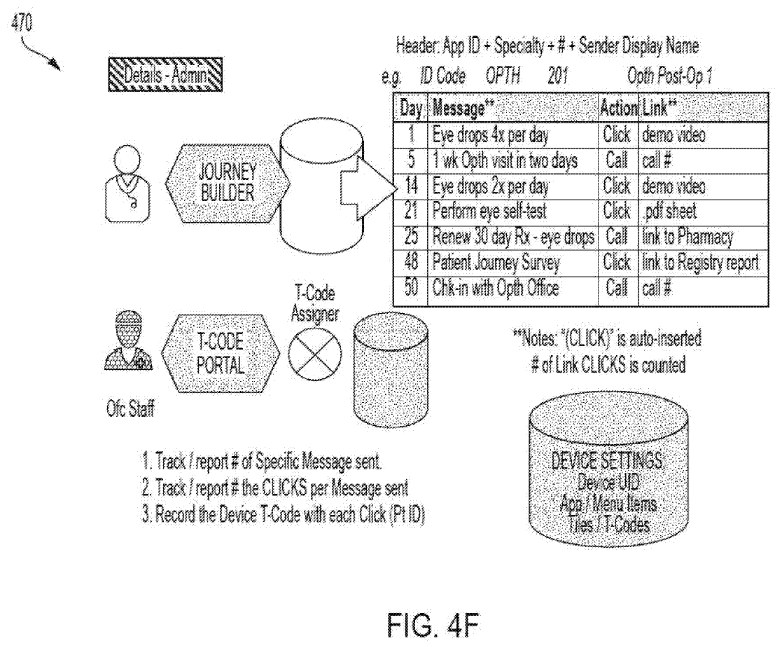

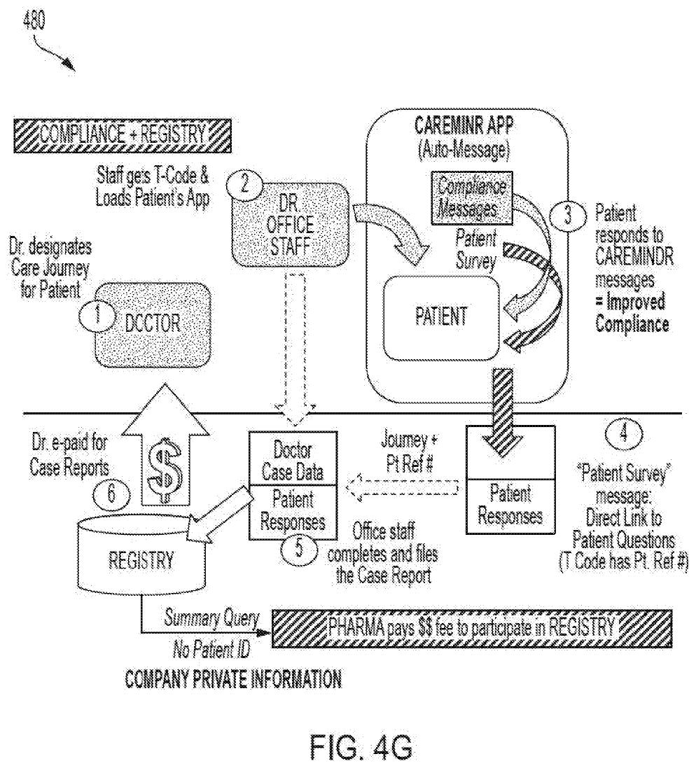

FIG. 4A illustrates an example setup configuration for a new course of treatment according to example embodiments. Referring to FIG. 4A, the illustration 400 includes the basic setup functions of linking a particular journey T-code (unique code) to the message and/or universal resource locator (URL) to link the application of the user to a customized template, such as a response form, questionnaire, etc. The unique T-code, date, time, response, and other records for each instance may be stored in a patient record managed by the application system database.

FIG. 4B illustrates an example database entry for the new course of treatment according to example embodiments. Referring to FIG. 4B, the example configuration 430 includes a database entry of messages which are organized by a category, in this case ophthalmology, and with a message content, including a link to a response page. The context and add-ons of a particular message may be customized based on a preferred layout or a default layout.

FIG. 4C illustrates a flow diagram 440 configuration for the new course of treatment according to example embodiments. Referring to FIG. 4C, the flow diagram includes a daily batch of messages which are setup to be delivered to one or more assigned patients. The process begins with a trigger to send a message, such as a matured date or time. The process then continues to deliver additional messages once confirmation of delivery is made. If the message is delayed or the response required is not received, the message may be resent as a late message requiring immediate attention. The process may continue to cycle to identify whether any messages are outstanding or have not been confirmed.

FIG. 4D illustrates an example list of messages for the ongoing course of treatment according to example embodiments. Referring to FIG. 4D, in this illustration 450, the various messages intended for a particular patient are illustrated by date. FIG. 4E illustrates an example setup configuration for various courses of treatment according to example embodiments. Referring to FIG. 4E, the configuration 460 includes a menu of options along with a set of potential journeys the user may be assigned to manage the ongoing health care treatment plans for that user. The overview of treatment options and dates are included for reference purposes.

FIG. 4F illustrates an example set of details of an ongoing course of treatment according to example embodiments. Referring to FIG. 4F, the details of the administrator are shown to include a journey builder function based on certain parameters, such as an identification code, specialty, a number and a sender name. The number of messages, responses and actions are recorded to demonstrate the user's interaction with the application and the specific treatment plan(s).

FIG. 4G illustrates an example network configuration of the various third parties involved in the application operation and compliance according to example embodiments. Referring to FIG. 4G, the large-scale network of communications among the integrated platform 480 demonstrates the process initiating with the doctor's office establishing a journey for the patient and assigning a T-code (unique code). The patient's responses are identified along with links and references to third party message links and other information sources.

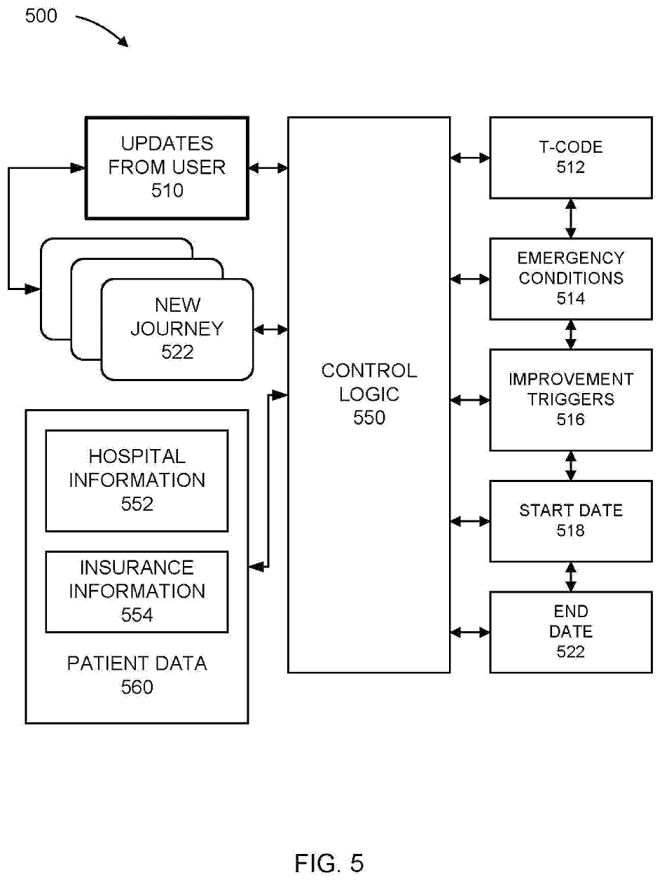

FIG. 5 illustrates a logic module configured to process the input and output parameters of the application according to example embodiments. Referring to FIG. 5, the control logic platform 500 includes a control logic unit 555, such as a processor or other processing entity that may receive updates from a user 510, new journey information 522 and/or patient data 560 including hospital 552, insurance 554, and other information. The logic may be configured to identify and link the unique T-code 512, emergency conditions 514, improvement triggers 516 for optimal changes to the treatment plan, along with dates 518 and new journey information 522 to perform the treatment plan.

In addition, while the term “message” has been used in the description of embodiments of the present application, the application may be applied to many types of network data, such as, packet, frame, datagram, etc. For purposes of this application, the term “message” also includes packet, frame, datagram, and any equivalents thereof. Furthermore, while certain types of messages and signaling are depicted in exemplary embodiments of the application, the application is not limited to a certain type of message, and the application is not limited to a certain type of signaling.

According to example embodiments, a user device, such as a smartphone, cellular phone, tablet device, laptop or other computing device with a memory and processor, may communicate with another computing device and/or a server to provide an integrated communication platform.

Example embodiments provide a computer system programmed to use automated messaging from medical offices to specific patients. The application is not limited to medical procedures and functions and may be used with other configurations for various purposes and services benefitting the end user. Example embodiments include three main computer systems, which work together in an integrated manner including a management platform that controls set-up, functionality, activity reporting, and messaging credentials for the users. An administrative platform that the doctor and doctor's office can access via the internet, and a mobile application that a patient can download into a mobile computer device such as a smartphone or tablet. The management platform acts as the nexus of the system sending outgoing messages on behalf of the healthcare provider and forwarding patient responses to the healthcare provider's administrative platform. The medical office may have a specific identification that is stored within the management platform.

The integrated platform provides a way of checking-in with a patient at prescribed intervals during times between office visits and when undergoing certain treatment that the doctor is providing or overseeing for the patient. The patient dialog may gather relevant information about the status of the patient's conditions or recovery and can be modified or tailored to specifically meet the dialog requirements of the treating physician. Once initiated by the doctor's office, the application operates in an autonomous manner by delivering messages to the patient to prompt responses if needed. The application functions are monitored to assure that the patient replies to the information requests from the doctor, otherwise a no-response alert is sent to the doctor's office. The interactions are recorded and time-stamped, providing an auditable record of the dialog, suitable for insurance billing purposes. The application can also support biometric information from devices that measure certain body functions, such as diabetes glucometers, or blood pressure cuffs, or any sensory readable health care metric. The application may also create a longitudinal record of information for the patient to illustrate week-to-week trends.

Response Alert Tags

As has been stated earlier, this method and system is utilized when a patient visits a healthcare provider for an illness/condition which is diagnosed and treated. The treatment occurs over a period and is referred to as a journey. The system tracks a patient's progress along the journey for that illness or condition and solicits health information from the patient at clinically relevant intervals, across an extended time period to enable evidence-based medicine. The specific information sought, the intervals, and the time period duration apply to specific conditions or illnesses for which a specific patient is being treated.

This solicitation for patient information may take the form of queries sent to the patient for information and the responses to those queries are delivered to the patient's healthcare provider (e.g. physician). The patient's journey may have several waypoints occurring at the clinically relevant intervals. The responses to the queries at these waypoints are meant to determine a patient's progress and status and to present to the healthcare provider evidence upon which to conduct evidence-based medicine. The responses are collected by the system and measured against historical norms for the patient and/or expected answers for similar patients on similar journeys.

In the event of an unexpected response to a query at that waypoint, the response is treated as notable. Notable events may be considered non-urgent or may be considered urgent and/or emergent. This divergence from the expected response outcome is graded for severity or urgency. If the severity or urgency of the response exceeds a predetermined threshold for that patient for that journey for that illness or condition at that waypoint, an urgent tag may be created and sent to the healthcare provider. The grading may be one of an immediate medical action advisory, a follow-up advisory, a medical history review advisory, and the like.

The information requested in the query is sent in a structured format to allow ease in answering and the response data is delivered to the healthcare provider in a structured data format to facilitate ease in analysis and trend detection. As an example, the request may be sent in the form of an electronic communication via the application. Here, the host of the application may populate a user interface on a front-end of the application with a message, interactive buttons, menu items, options, tiles, links, selectable actions, and the like.

The response alert tag is a feature that “tags” certain responses provided by the patient as information that requires follow-up or special notice by the patient's healthcare provider. The tag may indicate a level of severity or urgency, thus alerting the provider to information that may need immediate medical action, additional follow-up with the patient or a specific review of the patient's medical history.

The tag may be communicated to the provider through multiple channels depending upon circumstance and urgency and in an immediate manner or in a weekly aggregated format depending in part upon urgency.

Workflow instructions may be electronically linked to a tag, so that the specific healthcare provider that reviews the data will have guidance about the actions to be taken when a tag appears and any escalation of clinical review that might be appropriate.

Each patient for each illness or condition is interacted with by the system at intervals which are relevant to that illness or condition and the queries are sent to determine the patient's progress or status. The received response to the query is measured against an expected response, and anomalies or offsets are noted. If these response anomalies or offsets are larger than a predetermined amount, an urgent or severe issue may need to be addressed. Thus, the response is tagged as an urgent tagged response and may be sent utilizing a priority delivery schedule, a priority delivery indicia on the response and may be made to a priority delivery list determined by the healthcare provider. The response may be tagged as non-urgent if the determined urgency level does not meet the predetermined urgency threshold of the patient for the health-related issue.

The structured format allows an overlap of queries so that the patient is not answering multiple identical queries at any one point in time. Additionally, the structured format allows the data to be collected and logged in a structured format and assembled for future review both by the practitioner and the patient to determine trends.

In one example a method, includes requesting via a cloud-based system from a patient response to a query and receiving the response to the health-related query, determining an urgency level of the response based on the patient health-related issue and tagging the response as an urgent tagged response if the determined urgency level exceeds a predetermined urgency threshold of the patient for the health-related issue.

The method also includes providing the urgent tagged response to the health provider, where the urgent tagged response may be sent utilizing a priority delivery schedule, a priority delivery indicia for the response and may be made to a priority delivery list.

The method may also include tagging the response as non-urgent if the determined urgency level does not meet the predetermined urgency threshold of the patient for the health-related issue.

If the determined urgency level of the response is such that it rises to the level of a medical emergency, then the primary care physician may be immediately notified as well as emergency services such as 911 and if deemed appropriate, dispatched to the location identified either by the patient or gathered from a location indicator in his mobile device. If the response is deemed critical, in situations where the primary physician is not immediately available, an emergency medical specialist may be placed in active direct communication with the patient. The system would make available to the first responder the query and response to provide context for the escalation.

The response may be graded as to the tagged urgency level of the response, where the grading is at least one of an immediate medical action advisory, a follow-up advisory and a medical history review advisory. A follow-on query may be sent based on the urgent tagged response to give the provider context to the urgent tagged response. As an example, if the patient responds that they have been to the emergency room (ER) that may trigger another set of queries about the ER visit to add context to the response. This second set of queries may determine whether the ER visit was related to conditions or illnesses related to the journey, or whether visit was for a condition unrelated to the journey, but still of interest to the healthcare provider.

In another example a cloud-based system links a mobile device and a healthcare provider server. The cloud-based system requests a response to a query from a patient pertaining to a health-related issue, receives the response to the query and determines an urgency level of the response based on the patient health-related issue. The system also tags the response as an urgent tagged response if the determined urgency level exceeds a predetermined urgency threshold of the patient for the health-related issue and provides the urgent tagged response to health provider.

The cloud-based system may receive via the mobile device a sensor signal provided by a medical device in response to the query. The medical device may be a blood pressure monitor, a glucometer, a pulse meter, a continuous positive airway pressure device, a heart monitor, an implanted medical device and the like.

The cloud-based system may receive via the mobile device an audio or text message indicating a medical distress condition in response to the query or may overhear the patient indicating a medical distress condition in conversations or texts in an unsolicited message.

The system may also interpret patient actions in regards to patient historical norms, such as, if the patient is overheard slurring his speech, he may be having a stroke, or if he is discussing that he has pressure in his chest or his left arm is numb, he may be having a heart attack. At this point the system may connect him directly to a medical specialist and take other appropriate action, such as determining his location and dispatching emergency services.

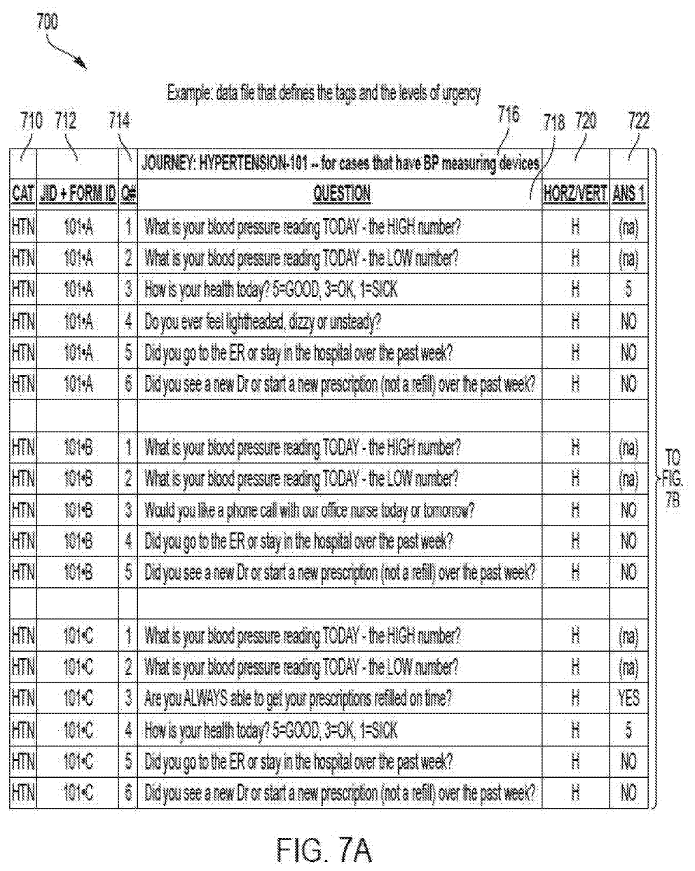

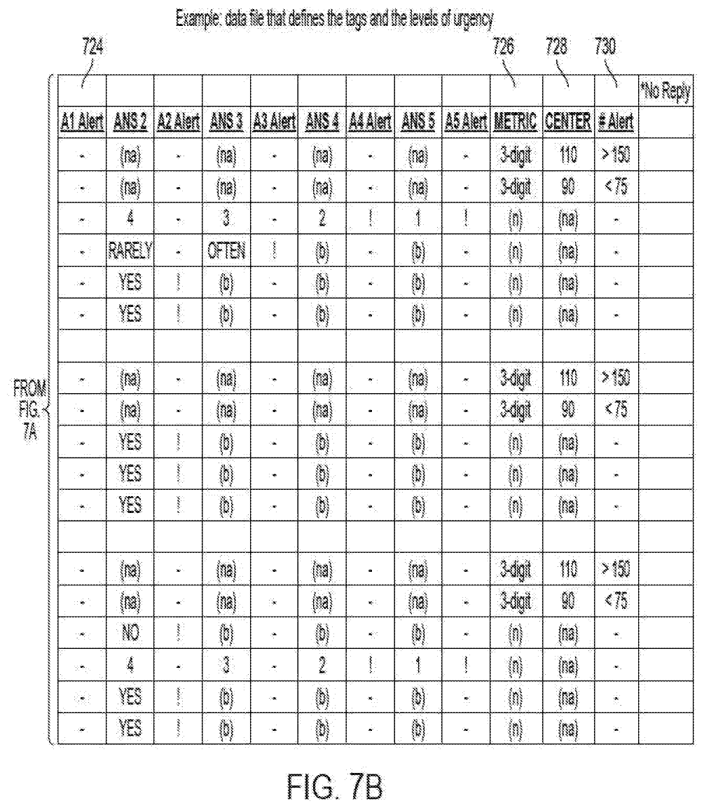

FIGS. 7A and 7B depict an example data file 700 that includes user answers to a set of questions and also tags that are generated in response to the user answers based on levels of urgency determined by the host system. The parts of the data file include a category of question 710, the journey ID and form ID 712, a question number 714, a patient journey for that specific illness or condition 716 and questions associated with that journey 718. The data layouts may be shown horizontally or vertically 720 which allows viewing of individual answers to queries. The historical and current query response 722 and the determined alert 724 are shown, which in a historical context allows detection of trends. The data output of the metric 726 may be numerical, yes or no, and the like, a median query answer 728 for that patient or that condition and an alert threshold 730 which may be modified by the healthcare profession are shown.

In the example of FIG. 7A, a first round of patient answers labeled as “ANS1” are received and analyzed by the host system which may be an application server. Here, the host system does not identify any matters that are urgent or otherwise need to be tagged. Thus, the column labeled “Alert A1” is empty. Meanwhile, in the example of FIG. 7B, a second round of patient responses labeled as “ANS2” may be received from a patient device and cause the host system to generate multiple alerts and corresponding tags. In particular, eight tags with the symbol “!” are generated and stored in a column labeled “Alert A2” in the data file 700 which is next to/adjacent to the column of data ANS2 which includes the patient responses. The host system may store this data file 700 in a back-end of a software application hosted by the host platform and may trigger messages, requests, responses, notifications, and the like, with different tags embedded therein to visually alert the reader as to the urgency of the message, etc.

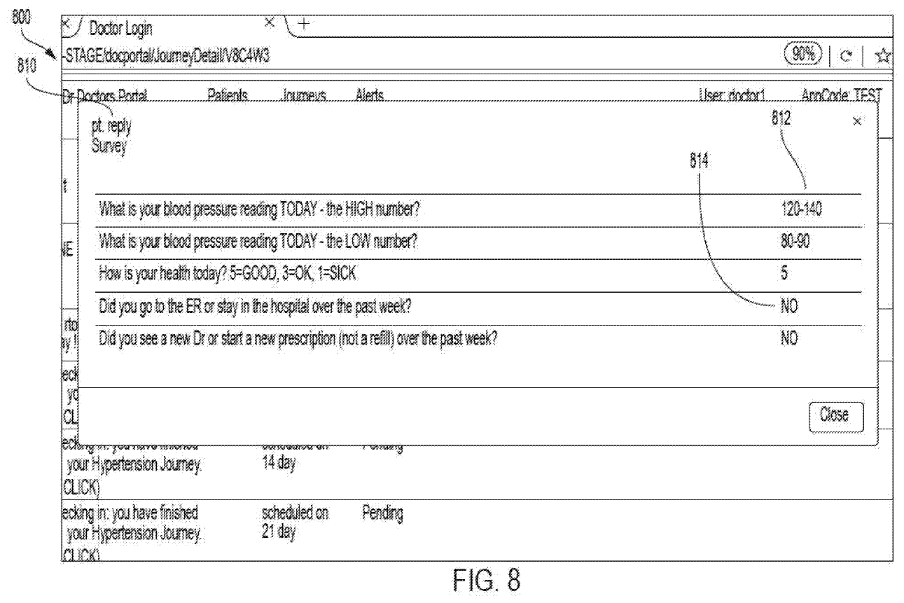

FIG. 8 depicts a patient survey reply 810 having a band of expected replies including a reply 812 for blood pressure, a reply 814 for whether the patient visited a hospital in the last week, and other replies including how the patient feels and whether the patient has started a new prescription.

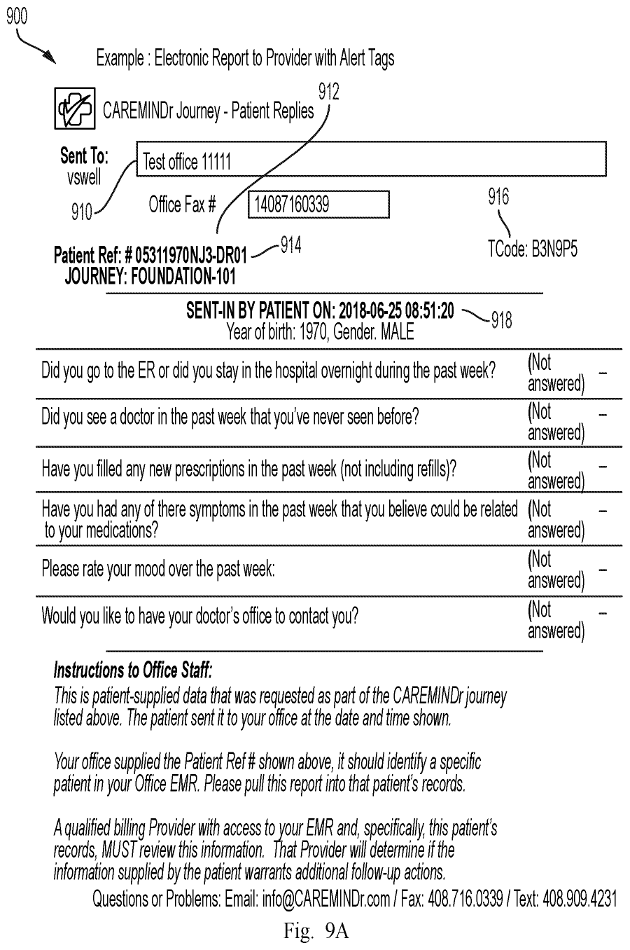

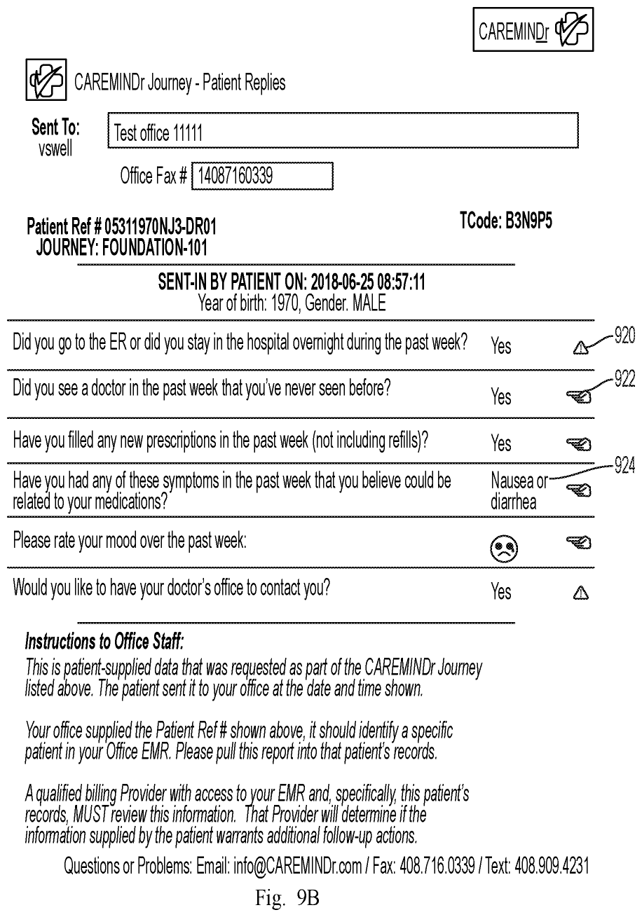

FIGS. 9A and 9B depict both a non-responsive reply set 900A and a responsive reply set 900B. The recipient 910, patient reference number 912, journey ID 914, unique T-Code 916 and timestamp 918 are depicted in both the non-responsive reply set 900A and the responsive reply set 900B. A responsive report having alerts indicates an urgent issue 920, a follow up item 922 and an emoticon 924 indicating a patient feeling is shown.

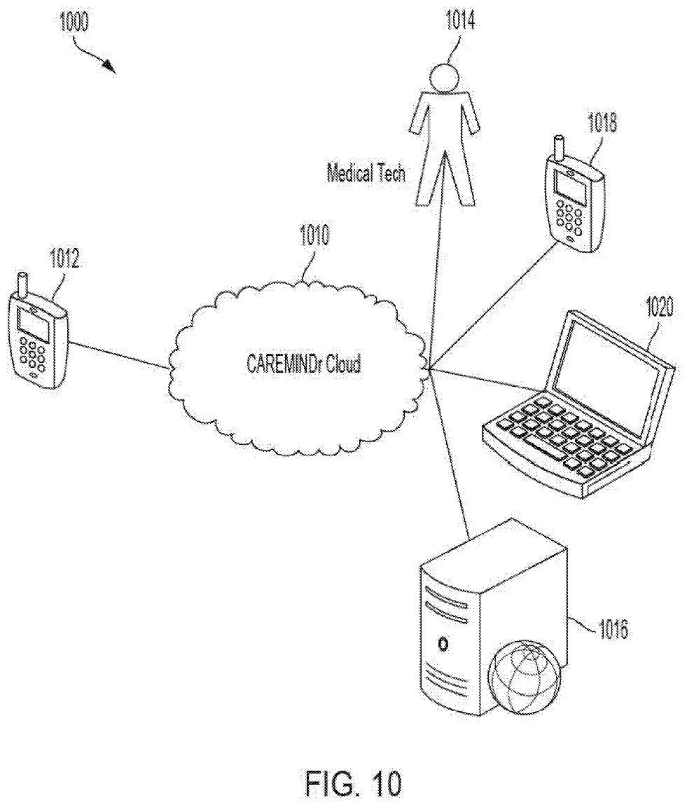

FIG. 10 depicts an example of a communication network 1000. The communication network 1000 includes a cloud-based system 1010 which sends a request with queries to a patient's communication device 1012 which the patient fills out and returns. The cloud-based system 1010 reviews the response and determines whether there are urgent or emergency issues and sends an urgent tagged response to the healthcare provider.

If there is an emergency issue the cloud-based system may contact or place the patient in contact with a medical technician 1014 in addition to notifying the healthcare provider by means of the healthcare provider's server 1016, the cloud-based system may issue a text or message to the healthcare provider. The communication route from the healthcare provider may be by means of mobile device 1018, a computer 1020, or the like. The cloud-based system 1010 may directly connect the patient via to the patient's communication device 1012 to the healthcare provider under appropriate circumstances. Non-urgent issues are sent to the healthcare provider for later review.

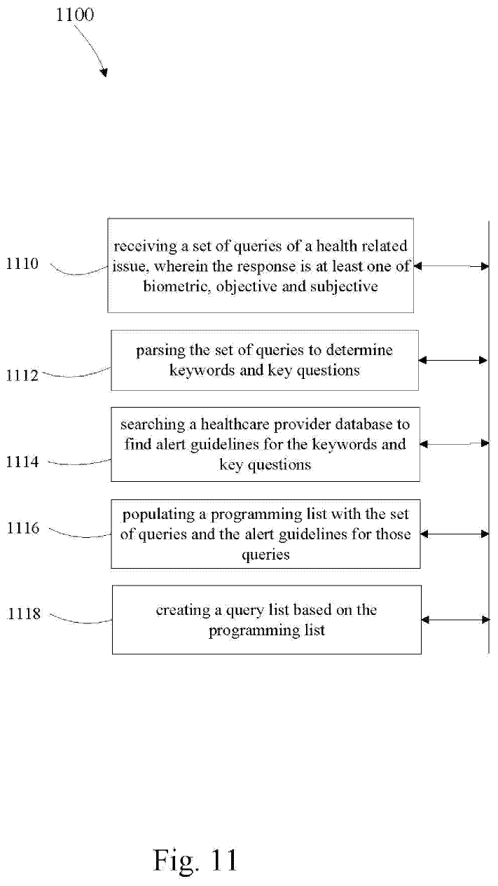

FIG. 11 illustrates a method 1100 related to building the query question platform in accordance with an example embodiment. Referring to FIG. 11, in 1110, the method may include receiving a set of queries regarding a health related issue, wherein the response is at least one biometric, objective and/or subjective piece of data. In 1112, the method may include parsing the set of queries to determine keywords and key questions. The method may also include searching a healthcare provider database to find alert guidelines for the keywords and key questions in 1114, populating a programming list with the set of queries and the alert guidelines for those queries in 1116, and creating a query list based on the programming list in 1118.

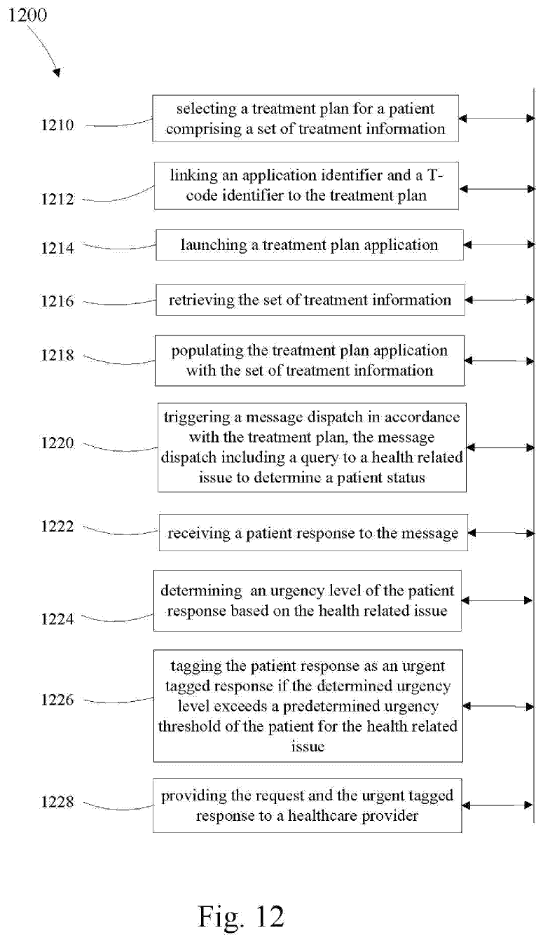

FIG. 12 illustrates an example of a method 1200 of tagging a message in accordance with an example embodiment. Referring to FIG. 12, in 1210, the method may include selecting a treatment plan for a patient comprising a set of treatment information, and linking an application identifier and a T-code identifier to the treatment plan in 1212. In 1214, the method may include launching a treatment plan application. In 1216, the method further includes retrieving the set of treatment information, populating the treatment plan application with the set of treatment information in 1218, and triggering a message dispatch in accordance with the treatment plan in 1220. The message dispatch includes a query to a health-related issue to determine a patient status and the system receives a patient response to the message in 1222. In 1224, the method may include determining an urgency level of the patient response based on the health-related issue, tagging the patient response as an urgent tagged response if the determined urgency level exceeds a predetermined urgency threshold of the patient for the health-related issue in 1226, and providing the request and the urgent tagged response to a healthcare provider in 1228. The method may also include proposing a set of workflow instructions linked to an urgent or non-urgent tagged response and presenting a clinical escalation review advisory if the response is tagged as an urgent tagged response.

With respect to the timing of patient responses, the method 1200 may also include, awaiting the patient response to the message for a late response duration and categorizing the patient response if the patient response is received within the late response duration. If the patient response is not received within the late response duration the method further comprises sending a duplicate message and flagging the patient response as non-responsive if the patient response to the duplicate message is not received within a second late response duration.

The timing of the message dispatches associated with the treatment plan is partly governed by a trigger table which may be embodied in tangible form as a file (e.g., an XML file, a JSON file, a spreadsheet file, an array, a database table, a document, etc.) As another example, the trigger table may be embodied as a queue where the individual messages are queued based on the order in which they are to be sent to the patient. As another example, the trigger table may be embodied in a cache. In some cases, the timing may be set using a time-to-live job or the like, which may be executed by the host system. In this case, the time-to-live job may include a pointer to the entry in the trigger table which corresponds to the message to be sent to the patient. The time-to-live job may include a timer that counts down or up until a threshold time. When the timer reaches the threshold time, the host platform may detect the expiration or the ending of the timer and send the corresponding message based on details included in the entry in the trigger table.

The method may include loading the trigger table with a set of entries having a set of trigger dates, respectively, based on a treatment plan of a patient. In this example, a message dispatch may transmit the set of messages according to the respective set of trigger dates. The method may further include receiving a message start date and receiving an initialization message from a patient mobile device to initiate the treatment plan and to initialize the set of trigger dates in the trigger table.

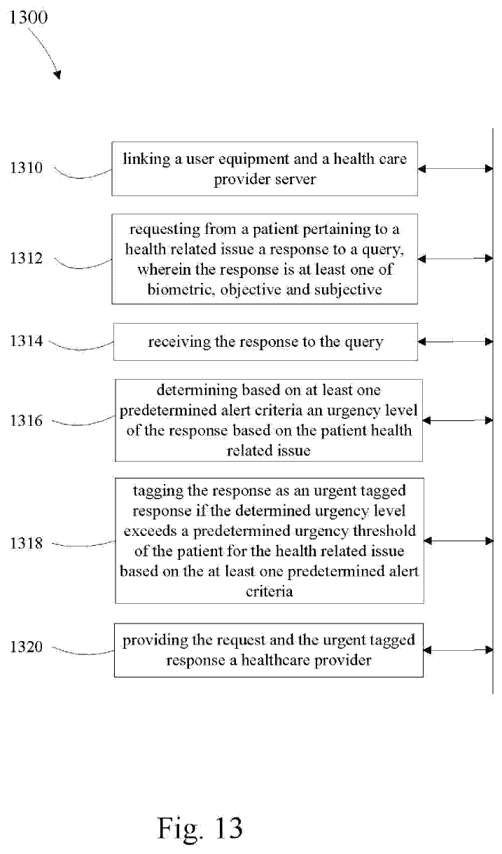

FIG. 13 illustrates a method 1300 associated with the tagging of responses in accordance with an example embodiment. Referring to FIG. 13, in 1310, the method may include linking a mobile device and a health care provider server, requesting from a patient pertaining to a health-related issue a response to a query in 1312, wherein the response is at least one of biometric, objective and/or subjective piece of data. Further, in 1314 the method may include receiving the response to the query. In 1316, the method may include determining, based on at least one predetermined alert criteria, an urgency level of the response based on the patient health-related issue. In 1318, the method may tag the response as an urgent tagged response if the determined urgency level exceeds a predetermined urgency threshold of the patient for the health-related issue based on the at least one predetermined alert criteria. In 1320, the method may provide the request and the urgent tagged response to a healthcare provider.

Most medical oversight is provided post-event in which a patient has been identified and is either currently in the throes of a medical condition or is recovering from a medical condition. In this example, a single patient is looked after by a group of people, this is a resource rich environment in which one patient has the attention of the medical community.

The example embodiments may provide a solution to issues currently present in performing medical oversight to a large community. In the example embodiments, the host system may be similar to a fire lookout tower in a forest. In the case of a fire lookout tower, a small number of people are looking after a vast number of trees within a large area of the forest. When a problem is detected by the lookout tower, personnel in the tower may trigger fire fighters to travel to that part of the forest to prevent a larger breakout. Likewise, the host system of the present application makes it possible for a small number of medical personnel to adequately look after an entire community. On a per person basis, this is a very medical resource restricted environment. In the example embodiments, the software application can assist the medical personal by identifying which patients need a communication today, and which can wait or which do not.

The COVID-19 pandemic has brought to the surface medical attention misallocation issues that exist in the current state of medical care. One issue is overtreatment and unnecessary treatment of patients, including office visits based on the calendar rather than current health needs, which is also described as low value care. Additionally, the pandemic has focused attention on elective procedures, which has subsequently shown that the performance of the elective procedure has had no demonstrable beneficial effect or reduced the recurrence of subsequent doctor visits. This low value care may be defined as medical actions in which the potential for harm far outweighs possible beneficial effects. Low value care also consumes doctor time and appointments on patients that do not require immediate attention, while taking away resources from patients with immediate needs, mostly because of a lack of awareness.

Another common misallocation of medical attention is in the current fee for service (FFS) model of medical payment —doctors are paid for specific work they do, unrelated to whether it is appropriate to a current patient need. While this FFS model, which requires doctors to perform certain tasks in certain very specific ways, may function in an age of a medically rich resource environment, it does not function well for all users, especially those in environments with fewer resources.

The example embodiments provide a solution that goes to the heart of the current medical misallocation issue by using an automated approach for periodically checking on and assessing all patients with a specific condition. This process will find that the majority of patients are fine and require no attention at this time. Further, it focuses medical attention on those patients that require it. The potential solution allows a resource sparse medical team to leverage the patient's inputs in conjunction with medically programmed and adjusted logic to direct focus on those patients needing immediate care, while continually keeping an eye on the remainder of the patient pool.

The questions in this possible solution are specific to the patient's condition, the responses may be numeric or multiple choice and may include one or more of biometric, objective, and subjective questions or requests. A biometric question would be one in which a request for blood pressure or other biometric data is requested and a number such as diastolic and systolic blood pressure are input, or a blood glucose level is input. An objective question pertains to facts such as “have you filled any new prescriptions in the past week?”, the answer of which is fact based and does not require patient interpretation or analysis. A subjective question would be one such as “please rate your mood over the last week” or “have you lost the sense of taste or smell over the last week?”. A request would be worded such as “would you like your doctor to contact you?”

The interpretation of the results is assembled in an array format that may be adjusted by the patient's doctor. The medical personnel in charge of the patient have the final say in how a response is interpreted and the medical weight given to the response. The triage is very simple, the highest alert level is the weight of the response, based on the input table any number or physician assigned emergency, critical, warning or nominal and tagged as such.

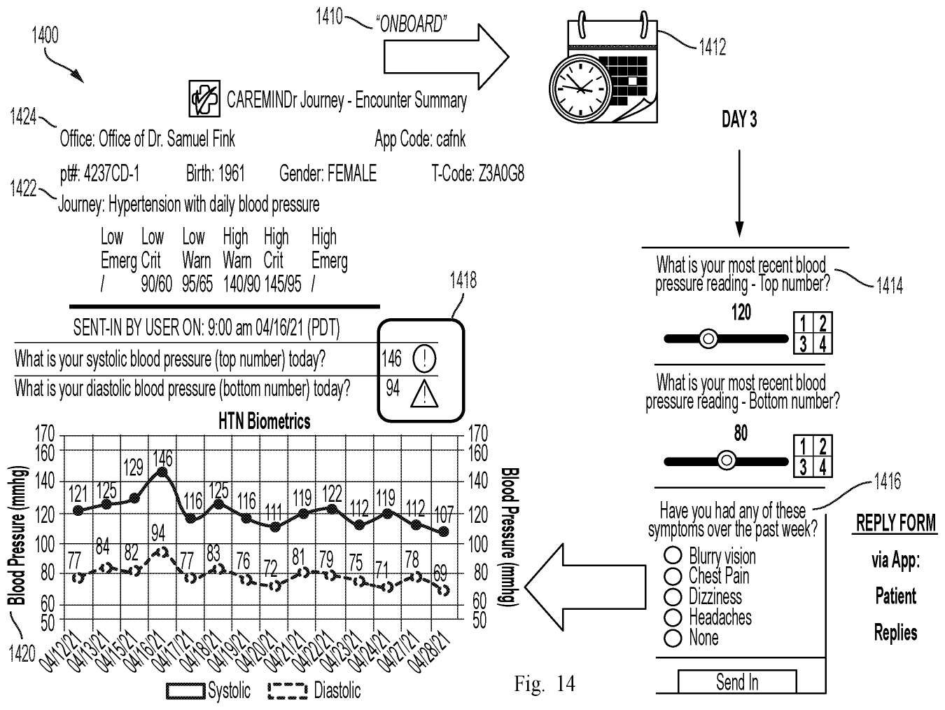

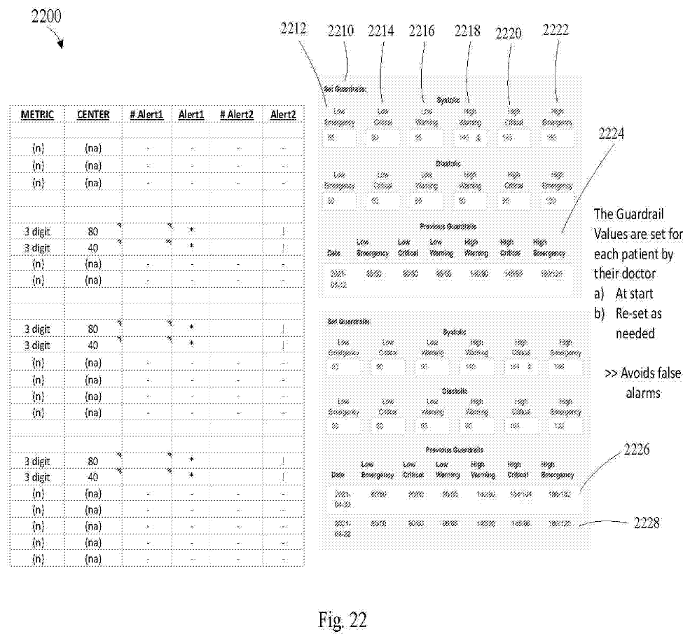

FIG. 14 depicts an example of an onboarding session 1400 with a patient. Referring to FIG. 14, patient data is loaded to a system or onboarded as indicated by item 1410 in FIG. 14 and a calendar 1412 is built within the application. The calendar 1412 is set up in this case for a journey for hypertension with daily blood pressure checks 1422 for a patient of a physician 1424. There are a series of questions on the journey and on day 3 the patient is asked for their most recent blood pressure top number, systolic, and bottom number, diastolic, via a message 1414, which would be described as biometric data as it is acquired from a device of some type, such as equipment used for reading blood pressure. The patient is also asked if they have had any symptoms over the last week via a message 1416, which would be considered an objective question. The patient's blood pressure readings are tallied and if the systolic and/or diastolic blood pressures are concerning, then they are flagged with a tag 1418 according to their severity and a biometric chart 1420 is provided for the healthcare provider. The exact criteria for an emergency, critical or warning for both systolic and diastolic blood pressure are shown in FIG. 22. These numbers are referred to as guardrails and may be reset by the medical staff to avoid false alarms.

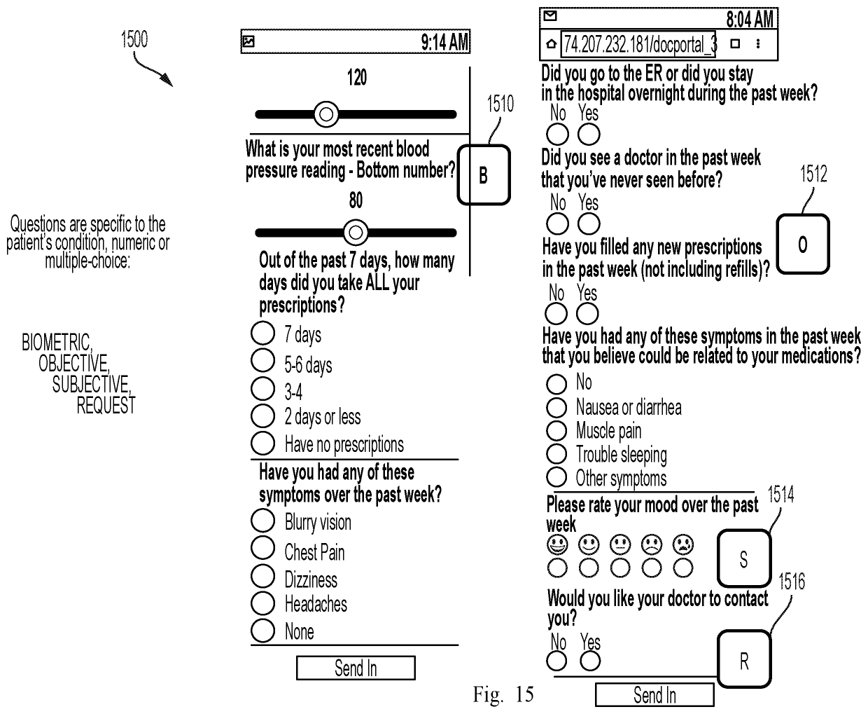

FIG. 15 depicts an example of a user interface 1500 with questions that may be asked of a patient embedded therein such as within content of a web page or a mobile application. In this example, these questions fall into one of three categories biometric, objective and subjective. Referring to FIG. 15, biometric questions 1510 are ones that have concrete device-provided data, such as blood pressure and fasting glucose levels. Since these numbers are captured by a device, and they are not subject to interpretation. Objective questions 1512 are questions asked of the patient that are also fact based, but not device derived. Fact-based questions have a correct answer. Examples of an objective question may include “out of the last seven days, how many days did you take all of your prescriptions?”, “Have you had any of the following symptoms over the past week?”, “Did you go to the ER or did you stay in the hospital overnight during the past week?”, “Did you see a doctor over the past week that you have never seen before?” and the like.

Subjective questions 1514 rely more on a patient's perceptions of the situation and not necessarily facts. Examples of subjective questions include “Please rate your mood over the last week” or “Have you lost either a sense of taste or smell over the last week”. Although subjective questions do rely on a patient's perception, it may indicate a specific medical condition or reaction to a medication. As another example, the patient may be asked for a request 1516. For example, the patient may be asked “Would you like for the doctor to contact you?”. This type of request is also considered by the system when assessing a patient. If a patient requests to see a doctor, there may be the concern of the seriousness of the condition that may not be directly assessed by the model and the questions asked.

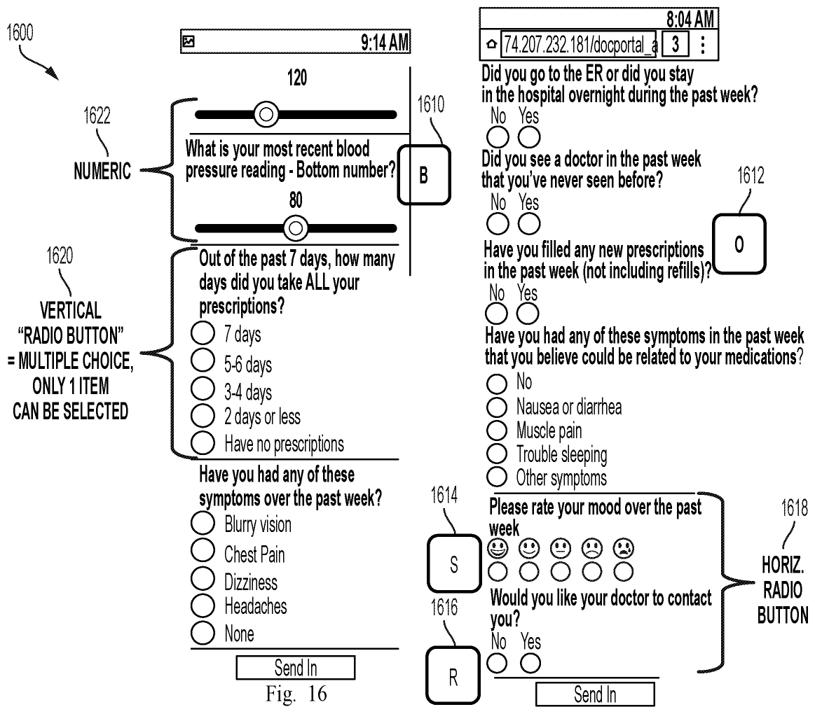

FIG. 16 depicts an example of a user interface 1600 that includes a layout of questions asked by the host system. Referring to FIG. 16, biometric queries 1610 are input numerically by the patient, who may use a slider 1622 to indicate the input. Objective questions 1612 asked of a patient may be vertically placed radio buttons if only one answer is allowed 1620 or may be side to side radio buttons in the case of a yes or no response. Subjective questions 1614 may be displayed and interacted with utilizing a horizontal radio button 1618. Requests 1616 having a yes or no response may be placed horizontally as side by side radio buttons.

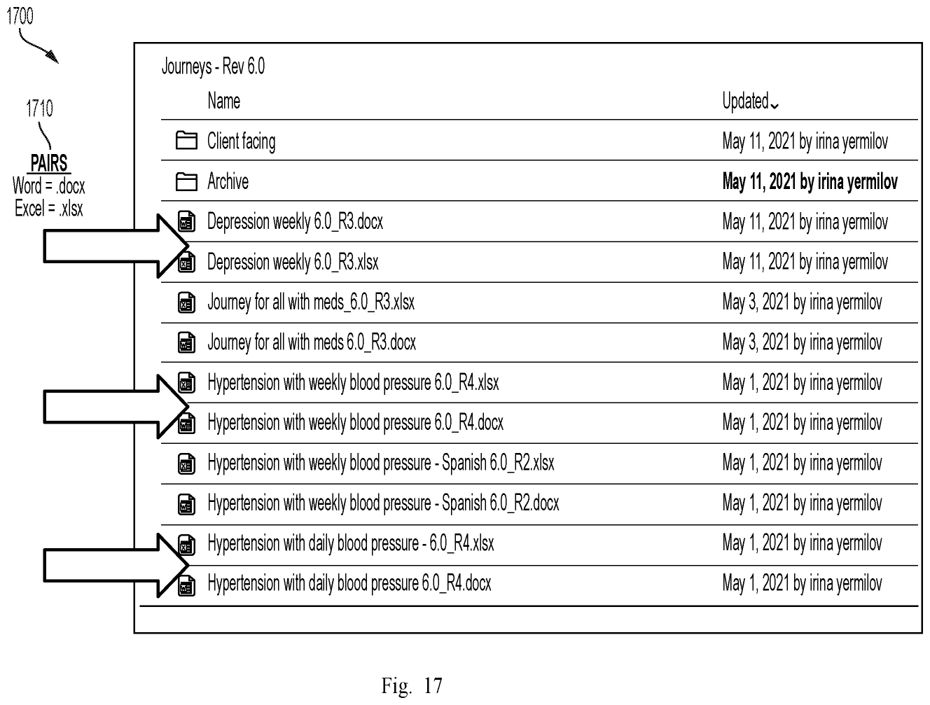

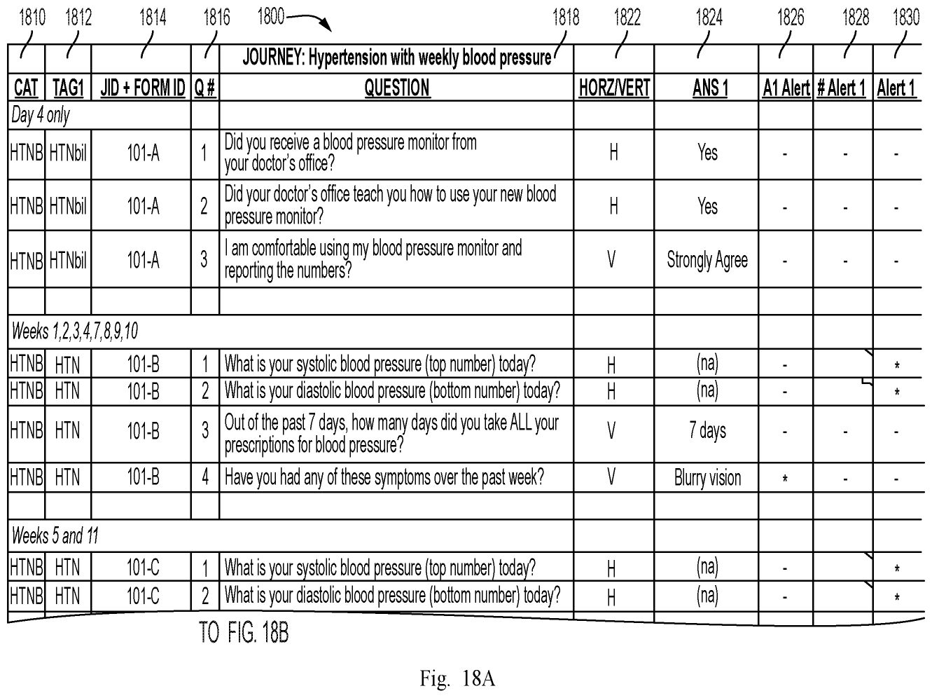

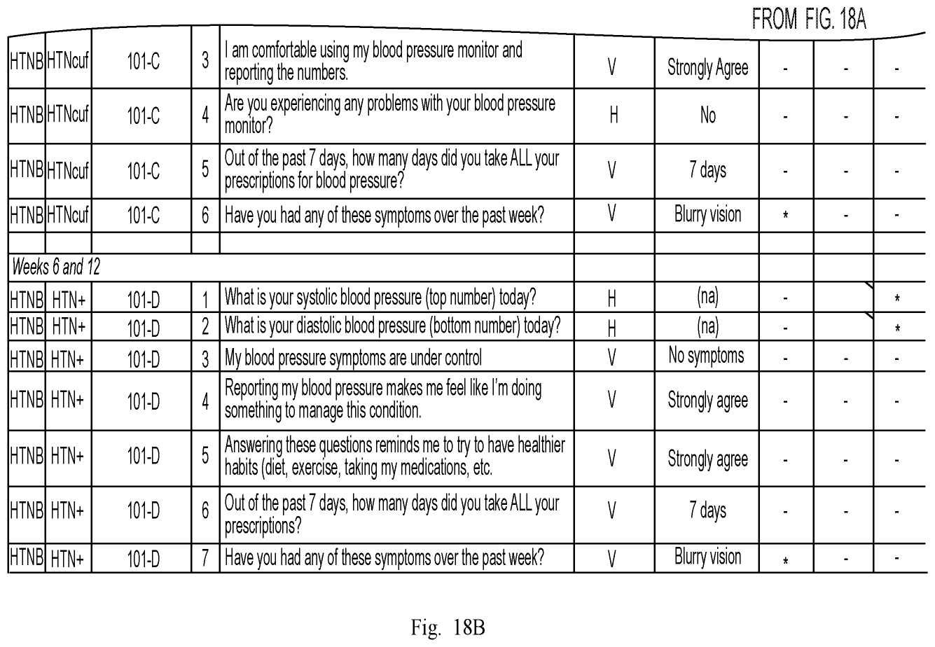

FIG. 17 depicts a display 1700 of a folder with a list of files used to program the host system. Referring to FIG. 17, a Word document and an Excel document are paired together in the list as shown at item 1710. The doctor will review the Word document to ensure that each of the questions asked and guardrails set are correct for their patient. The Word document information is translated into an Excel format for use by the system in setting up the specific patient journey. An example snippet from the translated Excel file is shown in FIG. 18.

FIG. 18 shows an example a spreadsheet 1800 file displayed via a user interface with some information for a “Hypertension and Weekly Blood Pressure Journey” 1818. The questions are grouped into four sets, including a first set with questions and responses/answers from day 4 only, a second set from weeks 1-4 and 7-10, a third set for weeks 5 and 11, and a fourth set for weeks 6 and 12. The spreadsheet 1800 includes a category column 1810, and a tag column 1812 which is broken into several types for this example representing different types of questions for the different weeks. A Journey ID and Form ID (JID+FORM ID) column 1814 is grouped with the four sets discussed above day 4 only being 101-A, weeks 1-4 and 7-10 being 101-B, weeks 5 and 11 being 101-C and weeks 6 and 12 being 101-D. The questions number 1816 are shown, and the question associated with the number is shown immediately to the left of the question number for that JID+FORM ID. Configuration data for displaying the questions may also be provided in the spreadsheet file 1800. For example, the direction of the radio buttons for the question is shown in a HORZ/VERT column 1822 and answer 1 is shown in an answer column 1824 with alert trip parameters shown in columns 1826, 1828, and 1830.

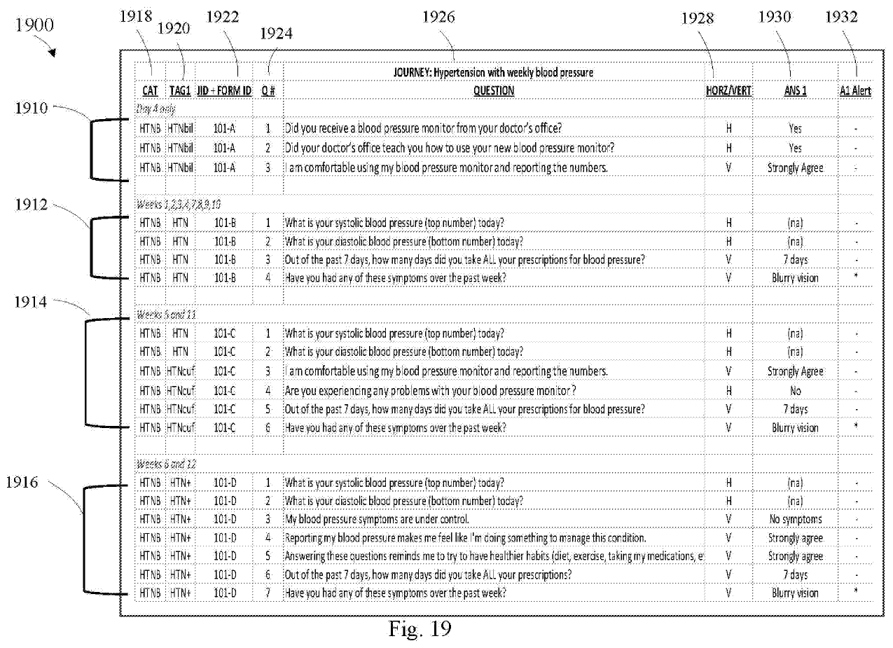

FIG. 19 shows another example of a spreadsheet file 1900 displayed via a user interface with some information for a “Hypertension and Weekly Blood Pressure Journey” 1926. The questions are grouped into four sets, including a first set 1910 from day 4 only, a second set 1912 from weeks 1-4 and 7-10, a third set 1914 from weeks 5 and 11, and a fourth set 1916 from weeks 6 and 12. The spreadsheet file 1900 also includes a category column 1918 and a tag column 1920, which is broken into several types for this example representing different types of questions for the different sets of questions. A JID+FORM ID column 1922 is grouped with the four sets discussed above 101-A for 1910, 101-B for 1912, 101-C for 1914 and 101-D for 1916. A question number column 1924 includes the numbering of the questions associated with the answers to the left of the question number for that JID+FORM ID. The layout of the radio buttons for the question is shown in a HORZ/VERT column 1928 and answer 1 is shown in column 1930 with alert trip points shown in column 1932.

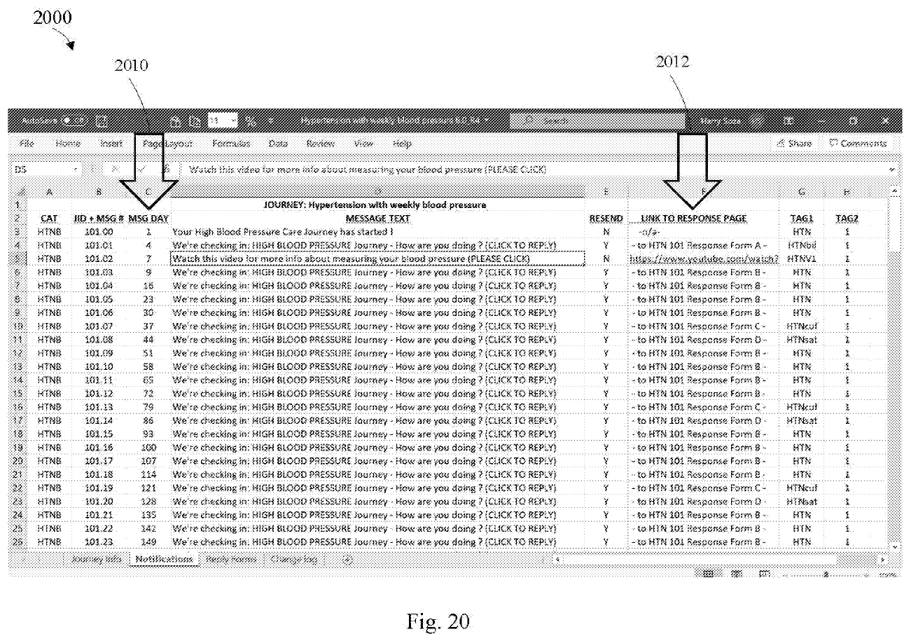

FIG. 20 shows an example of a spreadsheet file 2000 with message texts associated with the hypertension and weekly blood pressure journey. In this example, the texts are linked based on a category, a message number, and message day 2010, and a link to a response page 2012.

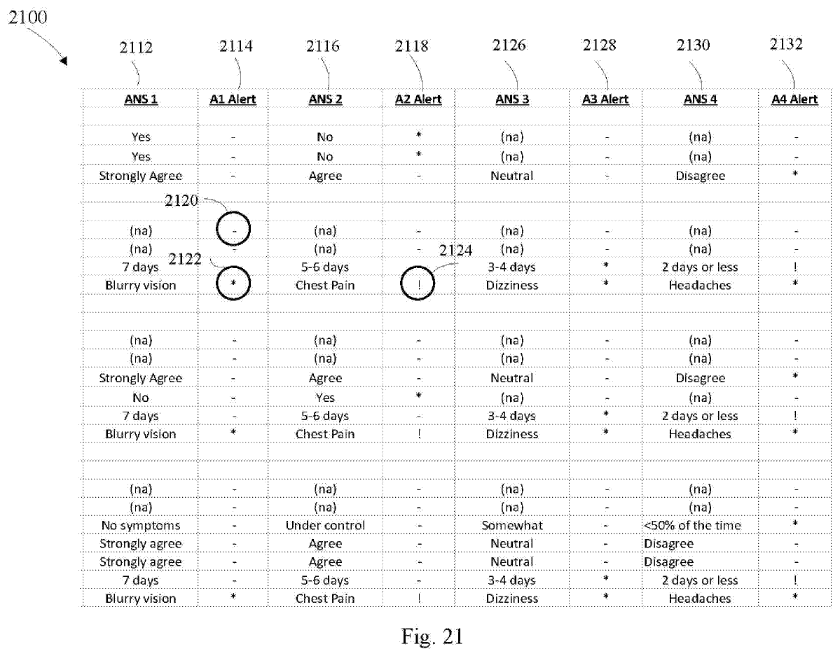

FIG. 21 depicts a portion of a spreadsheet file 2100 and indicates responses to particular questions in fields 2112, 2116, 2126, and 2130. The host system described herein may analyze the answers to the questions and fill-in an alert level with respect to each answer within the columns 2114, 2118, 2128, and 2132, which correspond to and are paired with columns 2112, 2116, 2126, and 2130, respectively. In this example, a tag 2120 includes a symbol “-” indicating a nominal alert, a tag 2122 includes a symbol “*” indicating a warning, and a tag 2124 includes a symbol “!” 2124 indicating a critical issue.

FIG. 22 depicts a user interface 2200 with guardrail values (e.g., alert thresholds, etc.) for blood pressure readings and their possible resetting by medical personnel. In this example the alert levels include “-” being nominal, “*” indicating warning and “!” indicating critical. A guardrails element 2210 includes fields for systolic blood pressure. These include an emergency-level low blood pressure value/threshold, a critical low blood pressure value 2214, a low blood pressure warning value 2216, a high blood pressure warning value 2218, a critically high blood pressure warning value 2220, and an emergency high blood pressure value 2222. These may be independently set for the systolic and diastolic blood pressures. The blood pressure alert level guardrails may be modified by the medical personnel and the dates for the modifications and values of levels are indicated in 2224, 2226, and 2228, to prevent false alarms.

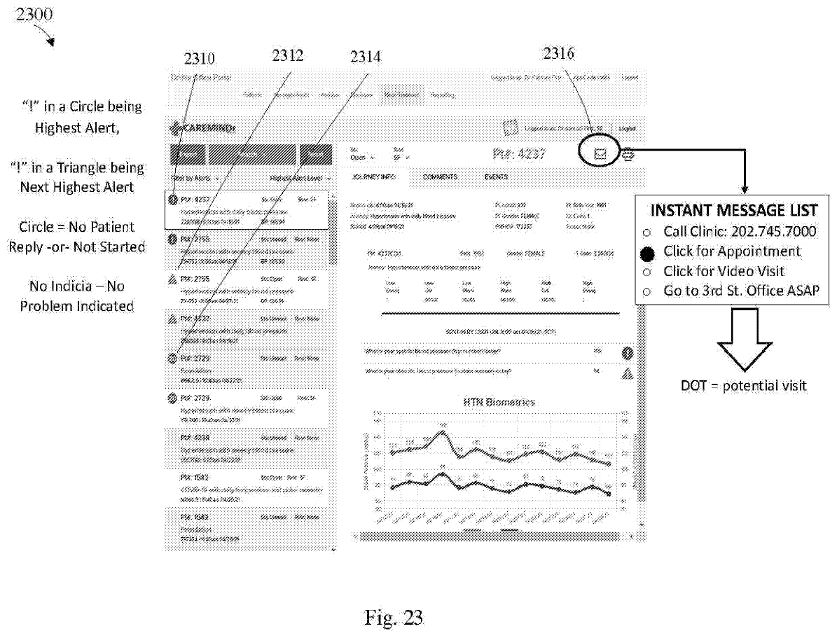

FIG. 23 depicts a review chart displayed via a user interface 2300 of a medical professional. In this example, the review chart includes a listing of a plurality of patients on a left-hand side thereof. Each patient may have an entry with a summary and details of the patient including an alert tag such as an alert tag 2310, which is displayed in the window. In this example, the software application may store the patients within the window by criticality. For example, patients that are in most critical condition may be rearranged to the top of the list while patients with less criticality may be lowered on the list. In this chart, the tags include a symbol “!” within a circle inside the patient summary which indicates the highest criticality (e.g., patient summary 2310) and a symbol “!” within a triangle is of next highest criticality (e.g., patient summary 2312). A circle without an exclamation point is an item to be reviewed by medical personnel (e.g., patient summary 2314), and responses without indicia indicate that no problem was detected. In this example the highest criticality case is for patient PT #: 4237 represented by patient summary 2310. In this example, a biometric history of the blood pressure values is shown in the bottom right-hand side of the screen and at the top right is a letter symbol 2316 that allows the medical personnel to send a message that may either ask the patient to call the clinic, click for an appointment, ask the patient to watch a video, or immediately go to a medical office. If clicked on, the application may trigger a corresponding action on the patient's instance of the software application. For example, in response to the doctor's input clicking on the letter symbol 2316, the application may send an instant message to the patient via the software application.

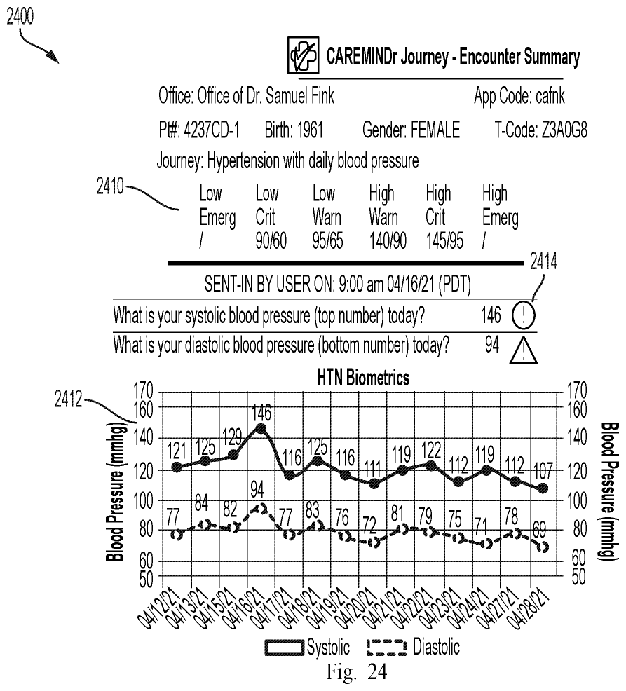

FIG. 24 is a user interface 2400 with a display of a review of the topmost critical patient from FIG. 23, patient PT #: 4237. In this chart the guardrails 2410 are identified, the biometric history 2412 is shown and the specific alert 2414 is shown. Here, the host system arranges the biometric data in the biometric history 2412 (which is a two-dimensional graph) to be displayed adjacent to a listing of the threshold values of the guardrails 2410. Furthermore, a display of any warnings may also be displayed including the specific alert 2414 along with the biometric data and the guardrail data.

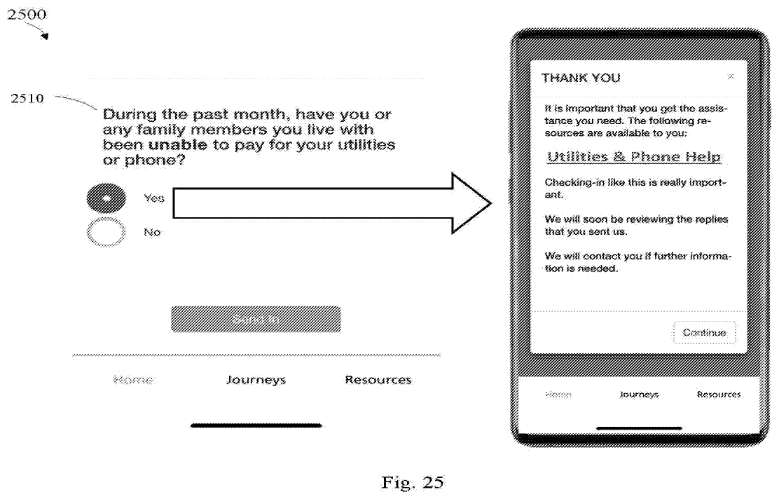

FIG. 25 illustrates a user interface 2500 with environmental condition questions that may have an impact on health issues. In this instance a question 2510 is asked, which wants to know whether the patient has been unable to pay utility bills or phone bills during the last month. The system then may identify a link and send the patient a link for help in paying the utility or phone bills. For example, the host system/application may send an instant message to the patient's instance of the application with a link therein that when selected by the patient, will navigate a user interface (of the application) from a current page to a payment page. The inability to afford to pay for services, see the doctor, or get to the medical office may affect a patient's health.

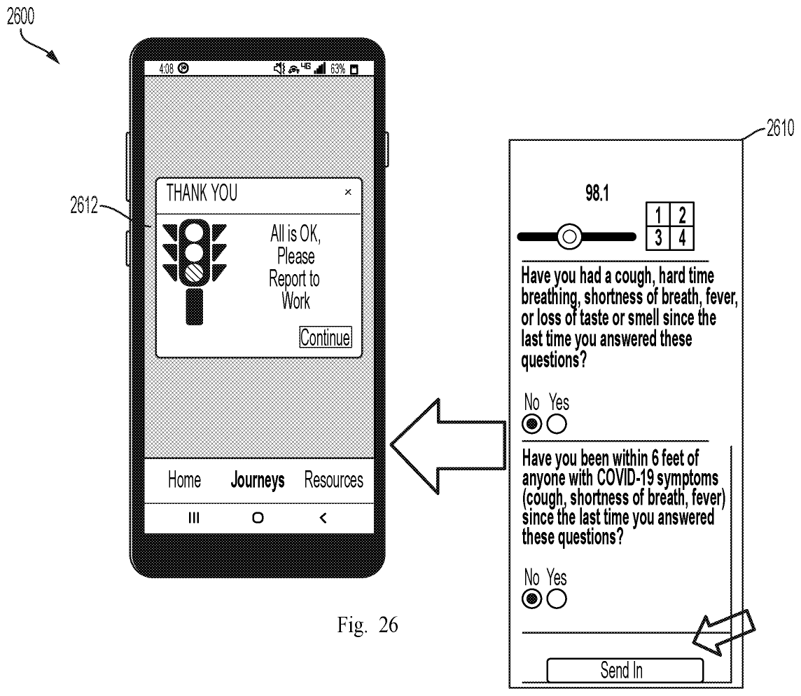

FIG. 26 illustrates a user interface 2600 with an example of questions related to spreading illnesses such as COVID-19 to fellow workers at a workplace. Here, the host system displays a message window 2610 on the patient/user device which asks a question for the temperature of a worker along with questions pertaining to cough, difficulty breathing, or loss of taste or smell and if they have been in contact with someone who has COVID-19. If the answers to the questions indicate a low risk having the disease the employee is instructed that it is okay to return to work via a pop-up window/display area 2612 that appears with an element such as a green light on a traffic light embedded therein.

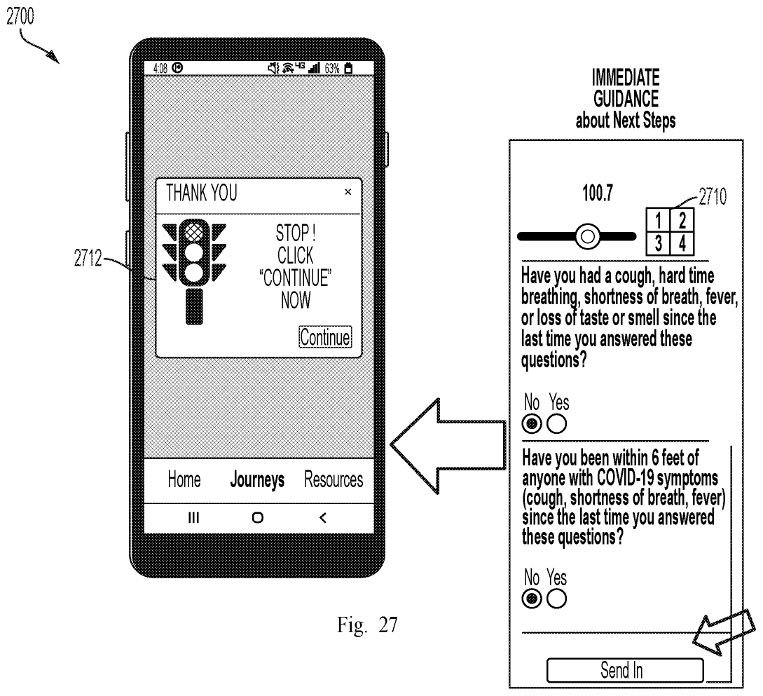

FIG. 27 illustrates a user interface 2700 with example questions related to spreading illnesses such as COVID-19 to fellow workers at a workplace. In this instance the patient has a slight fever of 100.7 as indicated by the slider input 2710 and is instructed to stop and press continue via a pop-up message 2712 which when selected displays additional instructions and protocols for handling a possible case of COVID-19.

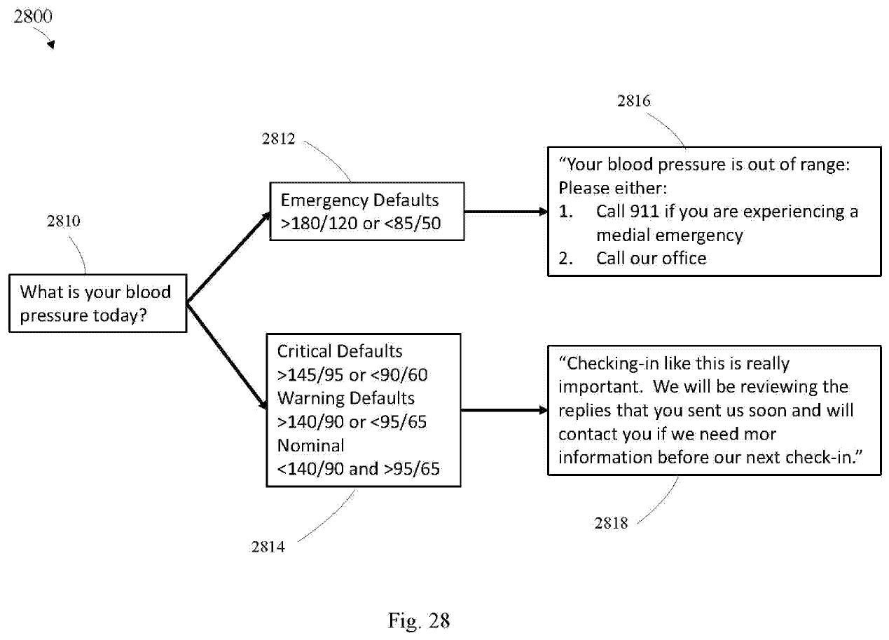

FIG. 28 illustrates a user interface 2800 with different sequences/branches of messages that can be generated and sent by the host system in an example of a hypertension and weekly blood pressure journey message flow for different blood pressures. In this example the patient is asked what their blood pressure level was today via a message 2810. The messages may be instant messages based on a client-server architecture in which HTML message content is transmitted. The host system may compare the response messages from the patient's mobile device (e.g., via instant message, etc.) and determine a level of urgency based on the response content. For example, an emergency may be indicated by reading levels above 180/120 or reading levels less than 85/50. The emergency thresholds/guardrails may be accessed by host system in 2812. For example, the host may access a table, a spreadsheet, a document, or other file that is accessible to the host system. The host system may also generate a response to the emergency which may include a suggestion to place a call to 911 or to immediately call a doctors office as noted in message 2816.

Other thresholds (guardrails) of other levels of urgency may also be compared to the message content (biometric reading) received from the user. As an example, the reading may be nominal if it is less than 140/90 and greater than 95/65. As another example, a warning urgency may be indicated if the reading is greater than 140/90 or less than 95/65 or critical at greater than 145/95 or less than 90/60 as indicated in the additional guardrails in file 2814. The system may send a response indicating that the data is being reviewed and that the office may contact the patient if more information is needed via an additional message 2818.

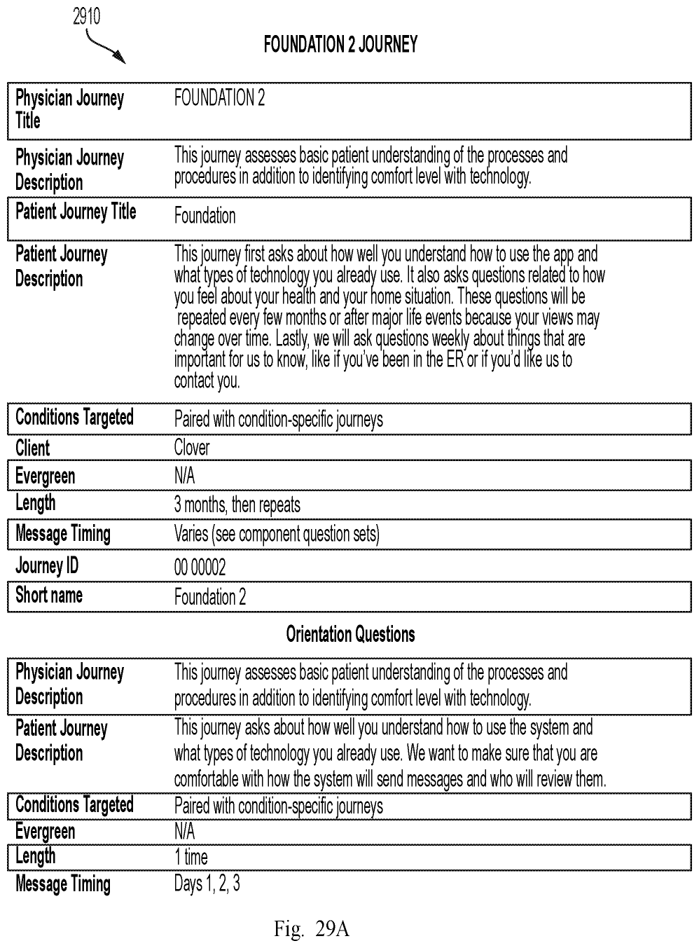

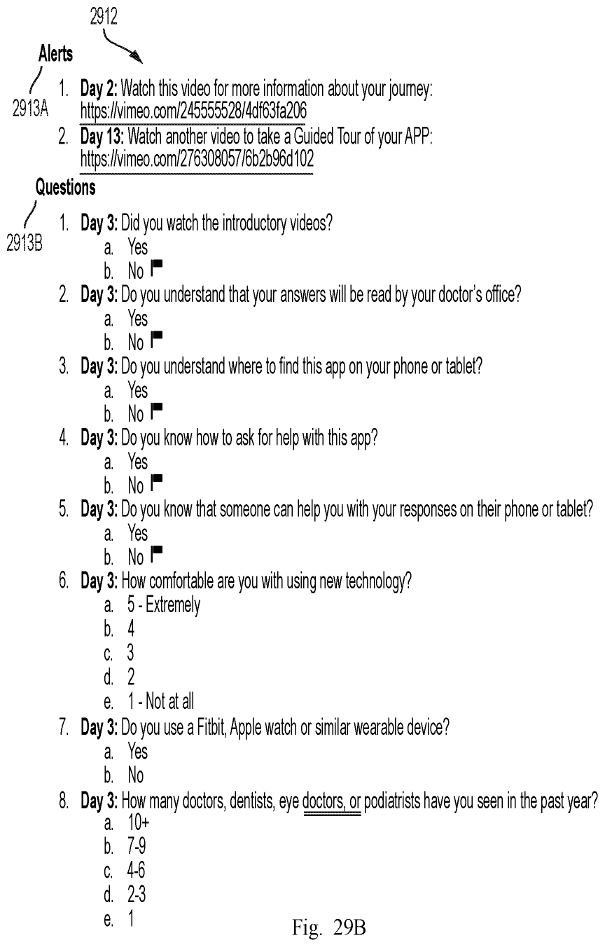

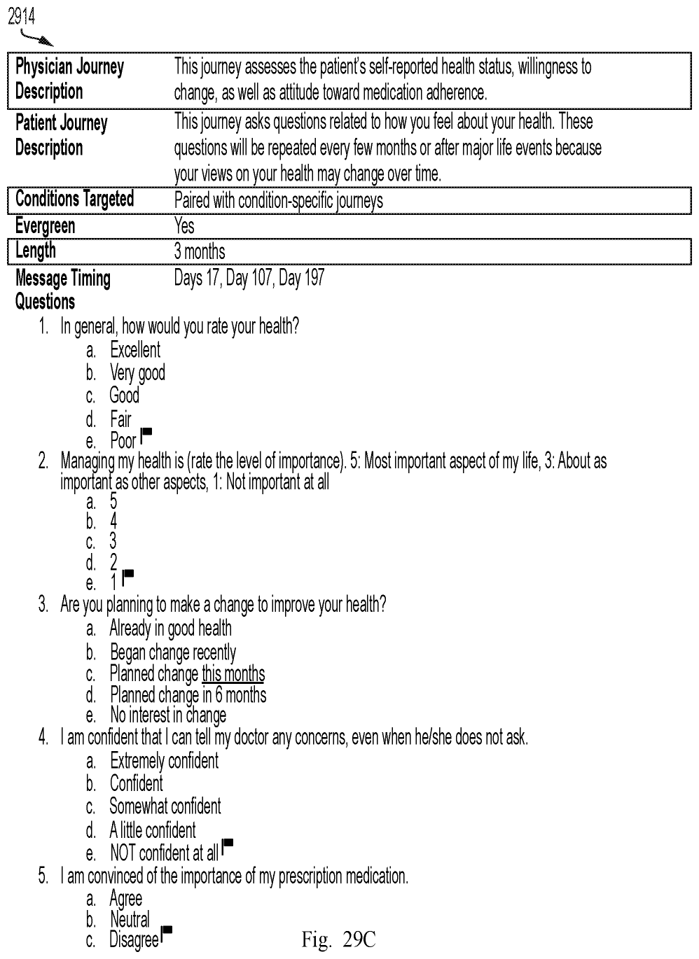

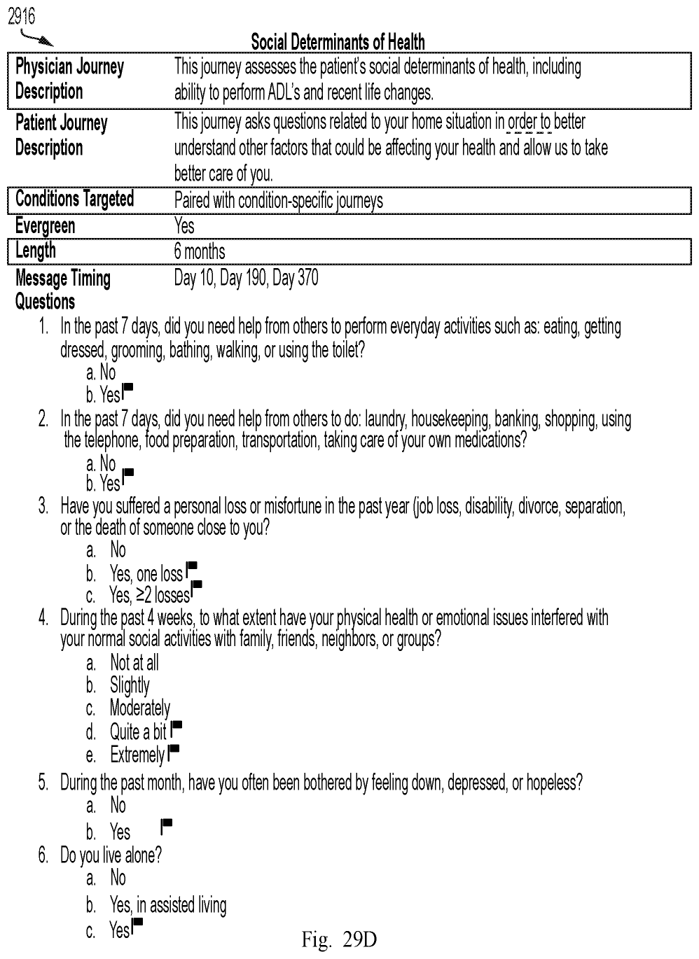

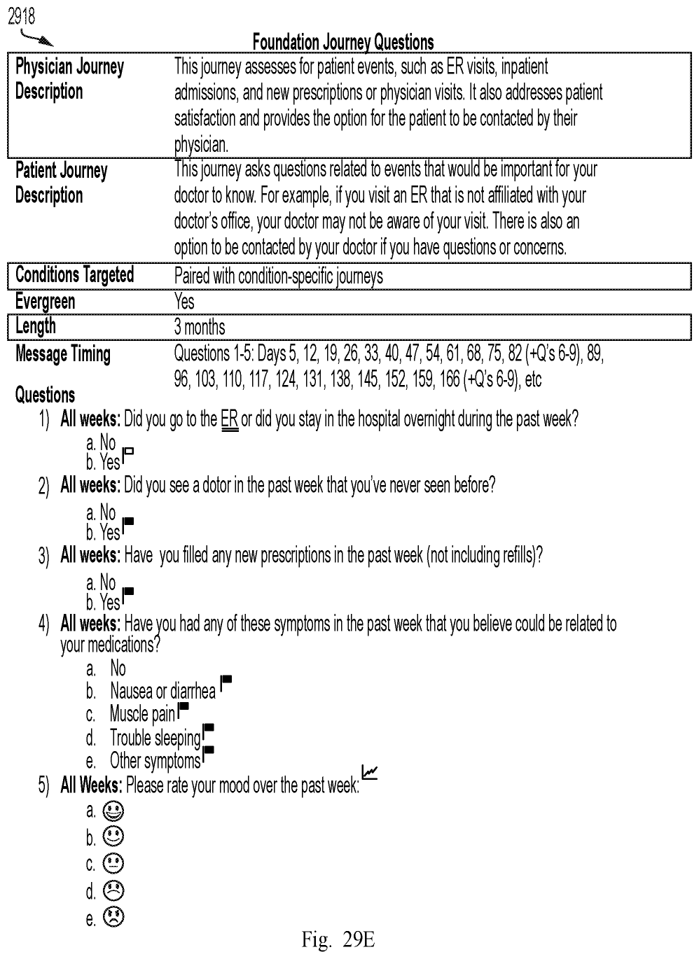

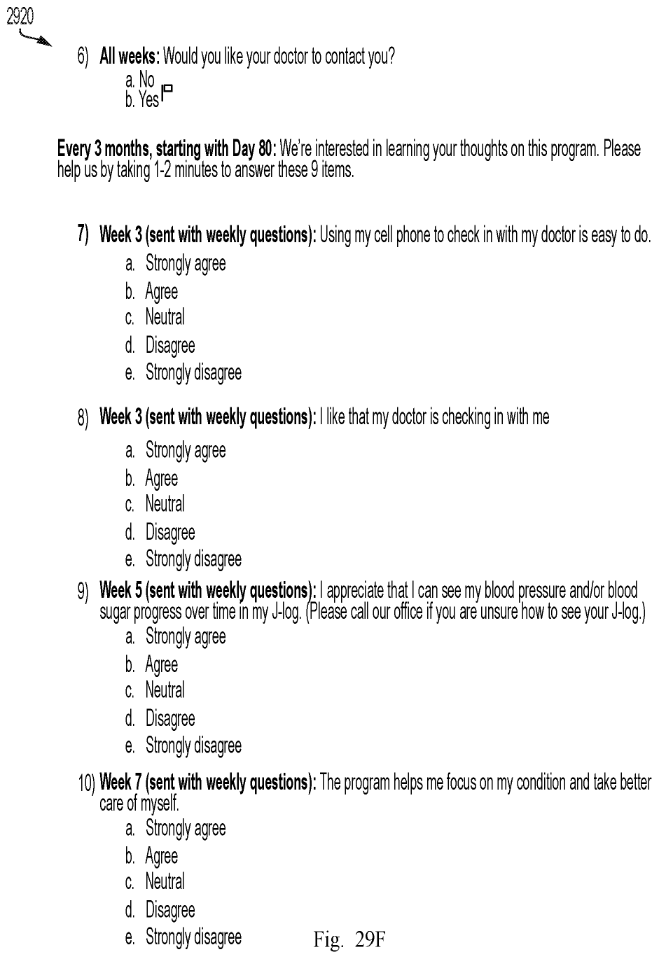

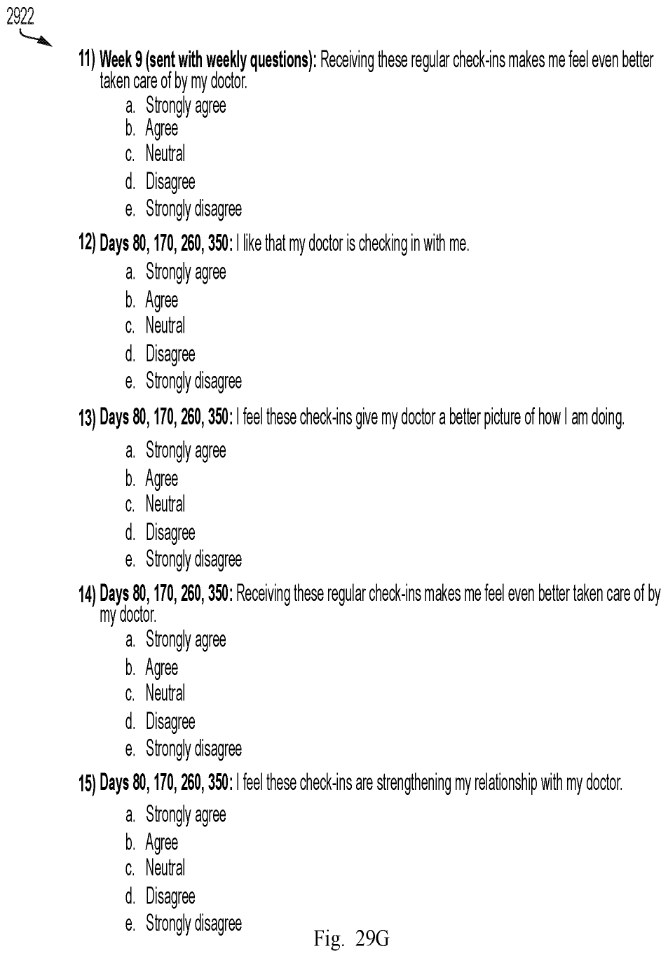

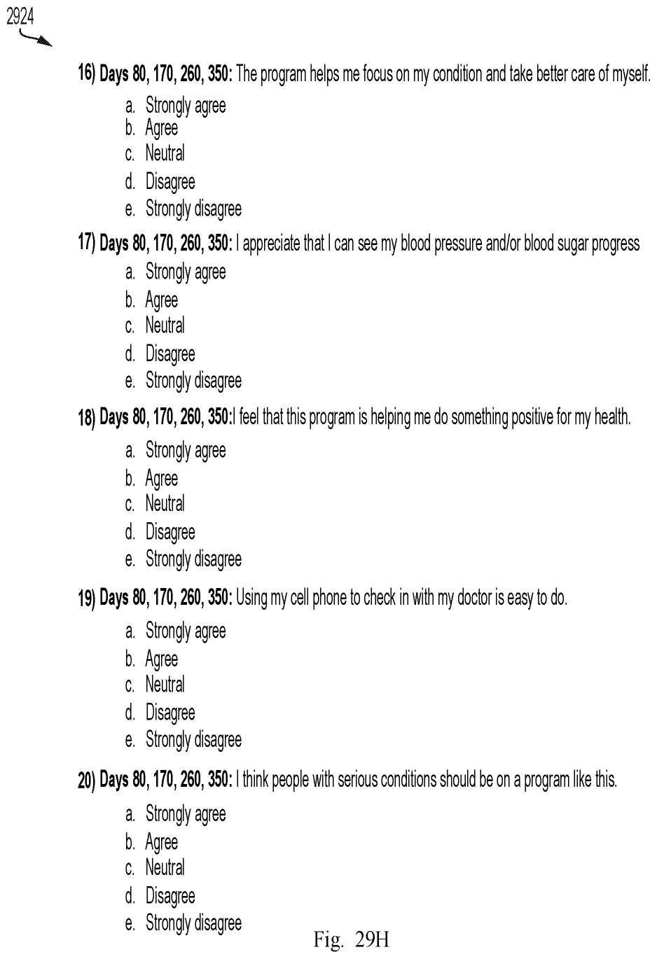

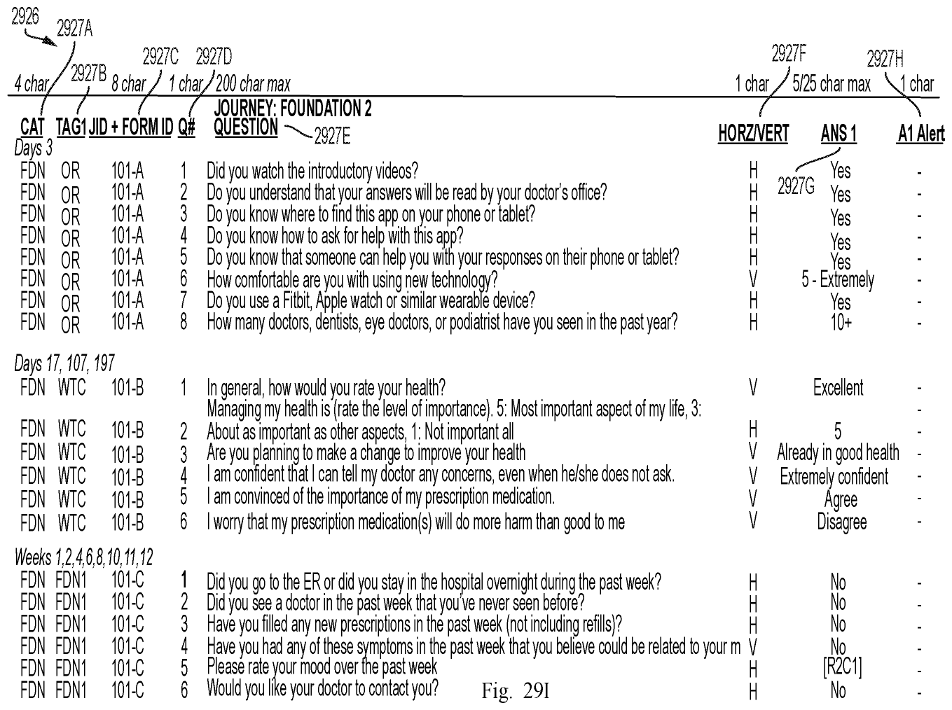

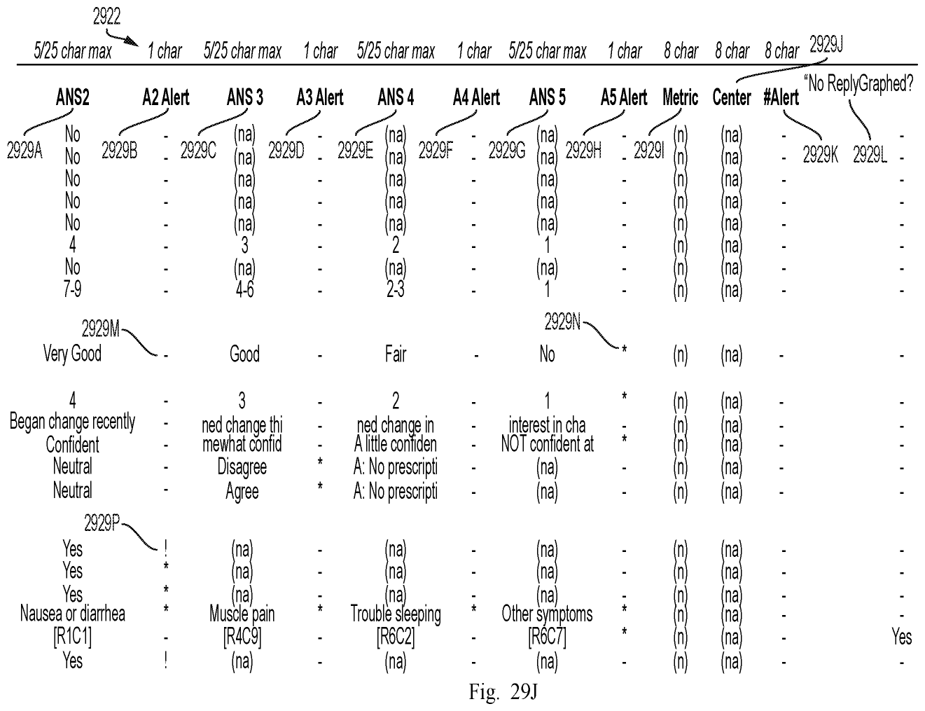

FIGS. 29A to 35D depict examples of journey queries for patients provided by healthcare providers and the subsequent building of the question set including alert guidelines.

FIG. 29A depicts an example of a page of a foundation journey 2910 that gives the health care provider a basic understanding of the processes and procedures of the journey and sets a baseline with respect to the patients' comfort with various types of technology currently used and an understanding of the application. The patient portion describes questions that will be asked and that are related to how the patient feels about their health and home situation. These questions will be repeated every few months or after major life events (e.g., medical events, age milestones, accidents, etc.) because patient views may change over time. Lastly, questions will be asked weekly pertaining to things that are important to know, such as whether the patient has been in the ER or if the patient would like to be contacted.

Example orientation questions pertain to about how well the patient understands how to use the system and what types of technology are already used. The foundation journey ensure that the patient is comfortable with how the system will send messages and who will review them.

FIG. 29B depicts an example of a foundation journey page 2912 that generates alerts 2913A for the patient about which videos to watch and questions 2913B that the health care provider may modify.

FIG. 29C depicts an example of a foundation journey page 2914 that includes an assessment of the patient's self-reported health status, willingness to change, as well as attitude toward medication adherence and asks questions related to how the patient feels about his health. These questions may be repeated every few months or after major life events because views on health may change over time.

FIG. 29C depicts an example of a journey page 2916 that includes questions into a patient's social determinants of health, including the ability to perform ADLs (activities of daily living) and recent life changes. This journey asks questions related to the patient's home situation to better understand other factors that could be affecting health.

FIG. 29D depicts an example of patient events questions 2918, such as ER visits, inpatient admissions, and new prescriptions or physician visits. It also addresses patient satisfaction and provides the option for the patient to be contacted by their physician. This journey asks questions related to events that would be important for the health care provider to know. For example, if a patient visits an ER that is not affiliated with the health care providers office, that the health care provider may not be aware of the visit. There is also an option to be contacted by the health care provider if the patient has questions or concerns. The example questions continue in FIGS. 29E, 29F, 29G and 29H, via journeys 2918, 2920, and 2922, respectively.

FIGS. 29I and 29J are examples of queries 2926 and 2928, respectively, which are built based on the healthcare provider input of FIGS. 29A to 29H. As is shown in FIG. 11, the inputs from FIGS. 29A to 29H are received and parsed and processed to look for key words and key questions, the database is searched for alert guidelines pertaining to those keywords and key questions and a set of queries are built.

FIGS. 29I and 29J are example question categories, tags, form-identification, question number, question, layout of the answers whether horizontal or vertical and answers and alerts associated with the patient answers and the graphing of the answers of the patient journey.

FIG. 29I shows an example of a spreadsheet file 2926 with some information for the foundation journey of FIG. 29A. A category column 2927A is indicated, a tag1 column 2927B is broken into several types for this example representing different types of questions for the different weeks. A Journey ID and Form ID (JID+FORM ID) column 2927C is grouped with three sets of questions including 101-A, 101-B and 101-C. A column for questions number 2927E is shown, and the question associated with the number is shown immediately to the left of the question number for that JID+FORM ID. The direction of the radio buttons for the question is shown in a HORZ/VERT column 2927F and an answer column (with answers to the questions) are shown in column 2927G adjacent with alert trip parameters shown in columns 2627H.

FIG. 29J depicts a portion of a spreadsheet file 2928 and indicates answers and alerts to particular questions of the foundation journey. Here, additional answers for additional rounds of questioning of the patient (e.g., repetition of the same questions every few hours or days, etc.) are shown in columns 2929A, 2929C, 2929E, and 2929G. In addition, alerts corresponding to the answers in columns 2929A, 2929C, 2929E, and 2929G, are shown in columns 2929B, 2929D, 2929F, and 2929H, respectively. The spreadsheet file 2928 also includes a column 2929I for metrics, a column 2929J for centering information, and a column 2929K for alert accumulation information. Also included is a column 2929L which indicates whether a reply was not received.

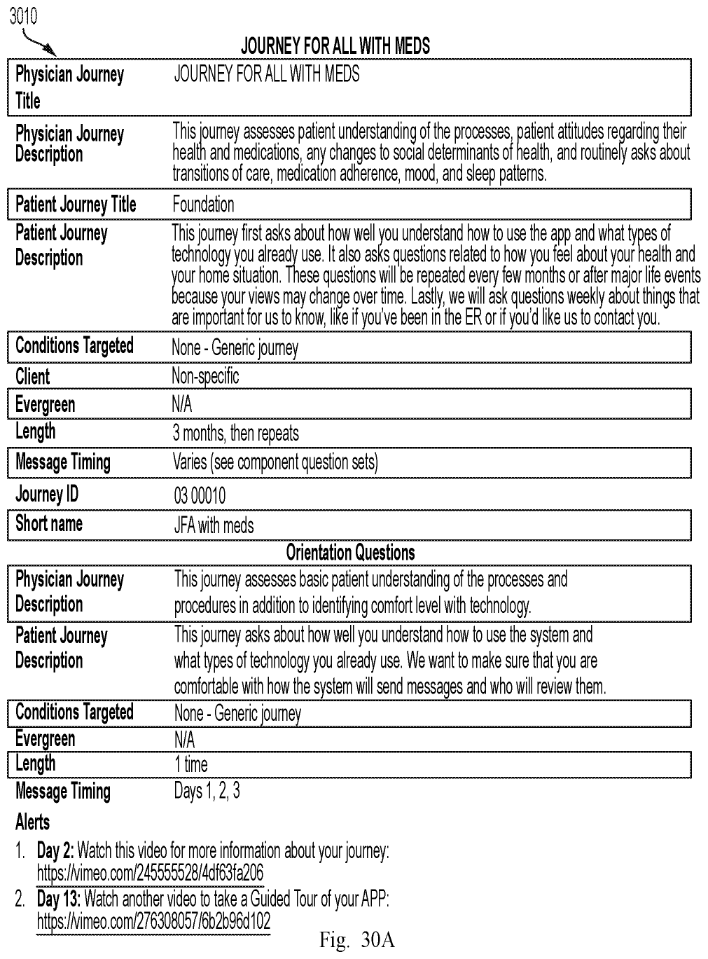

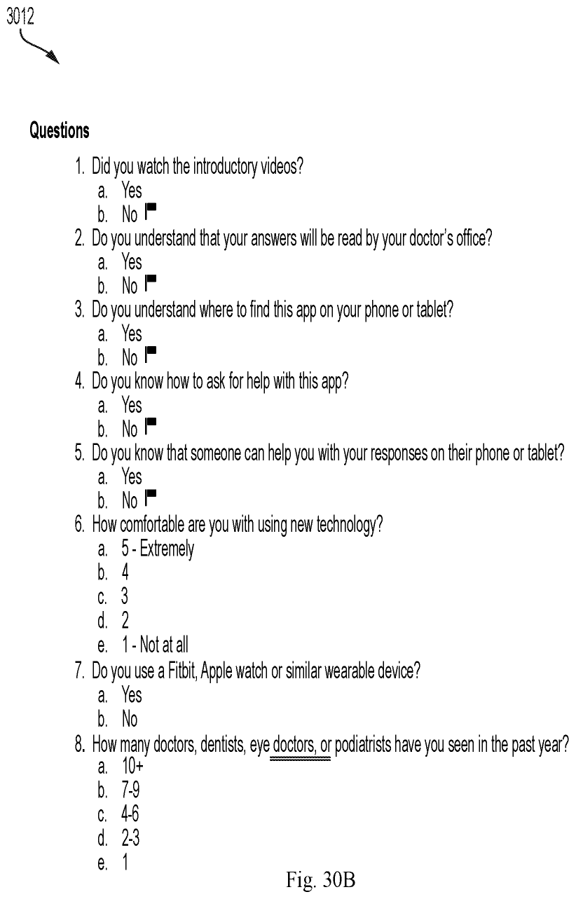

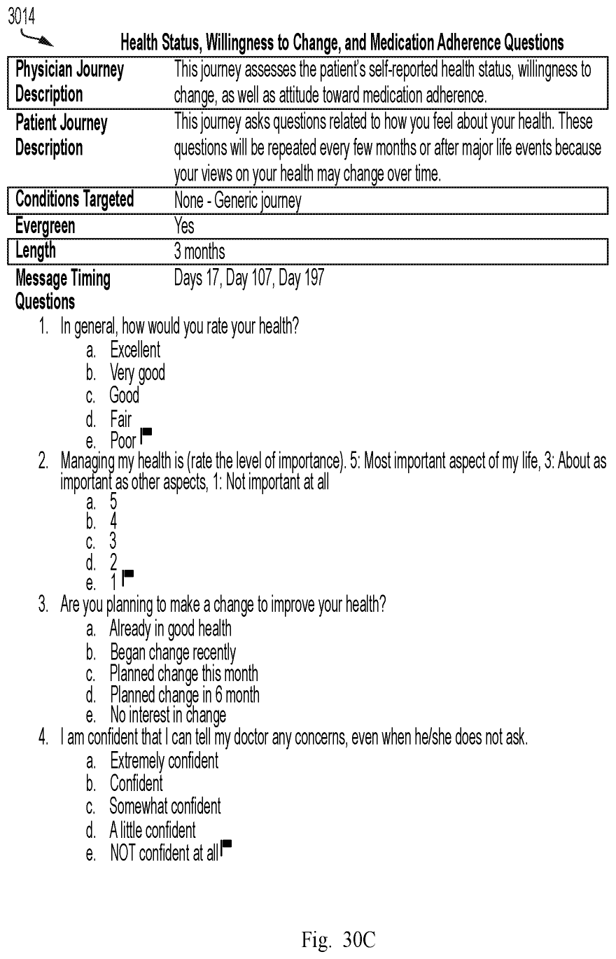

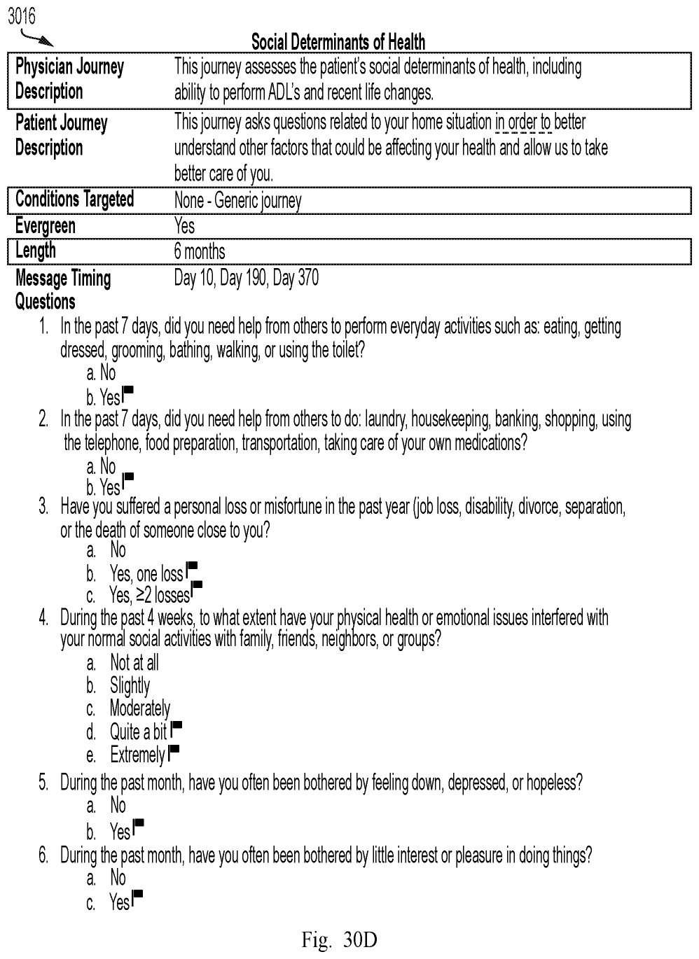

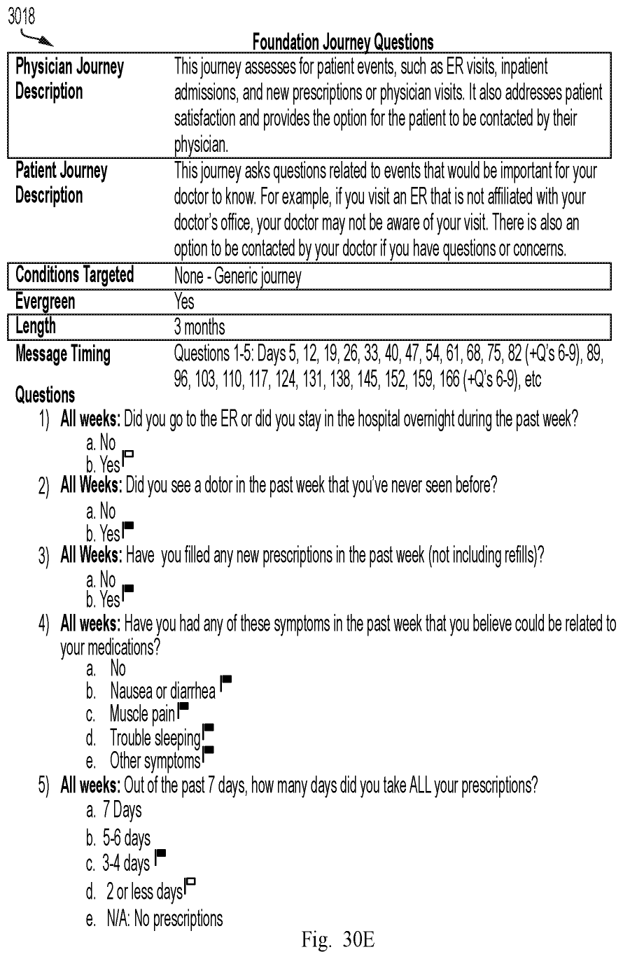

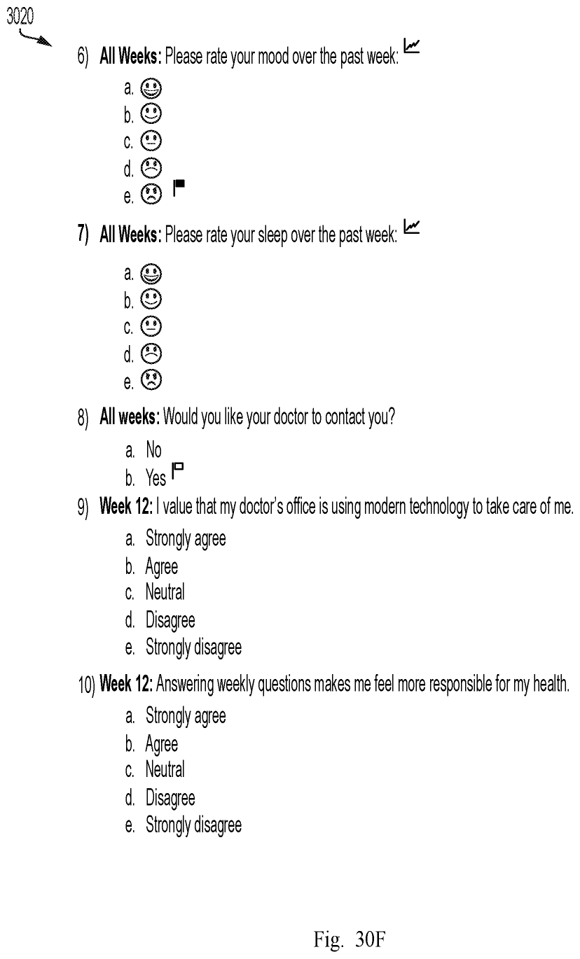

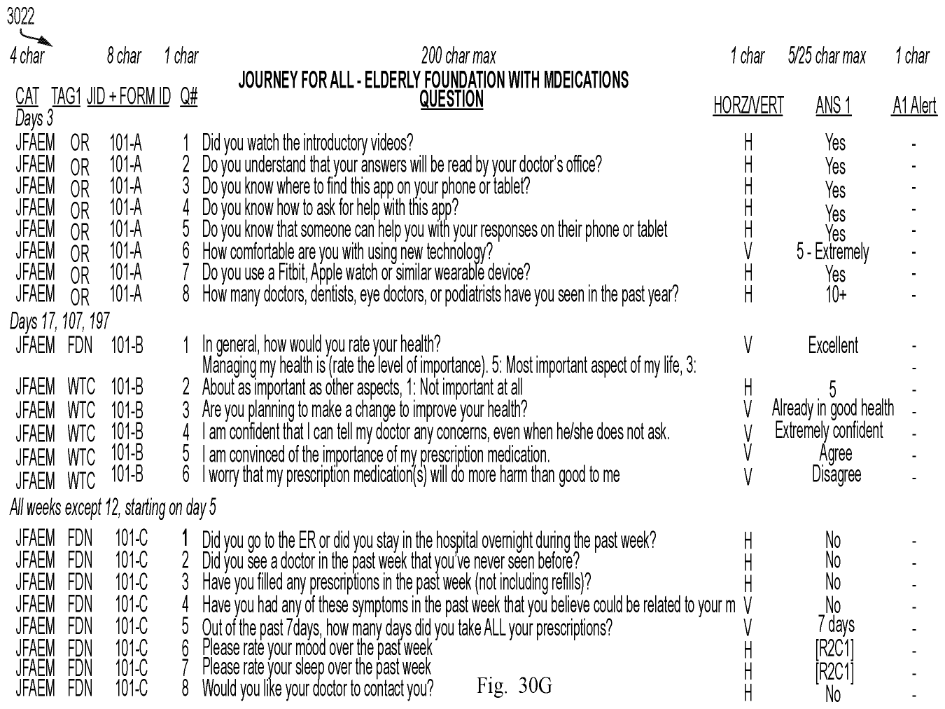

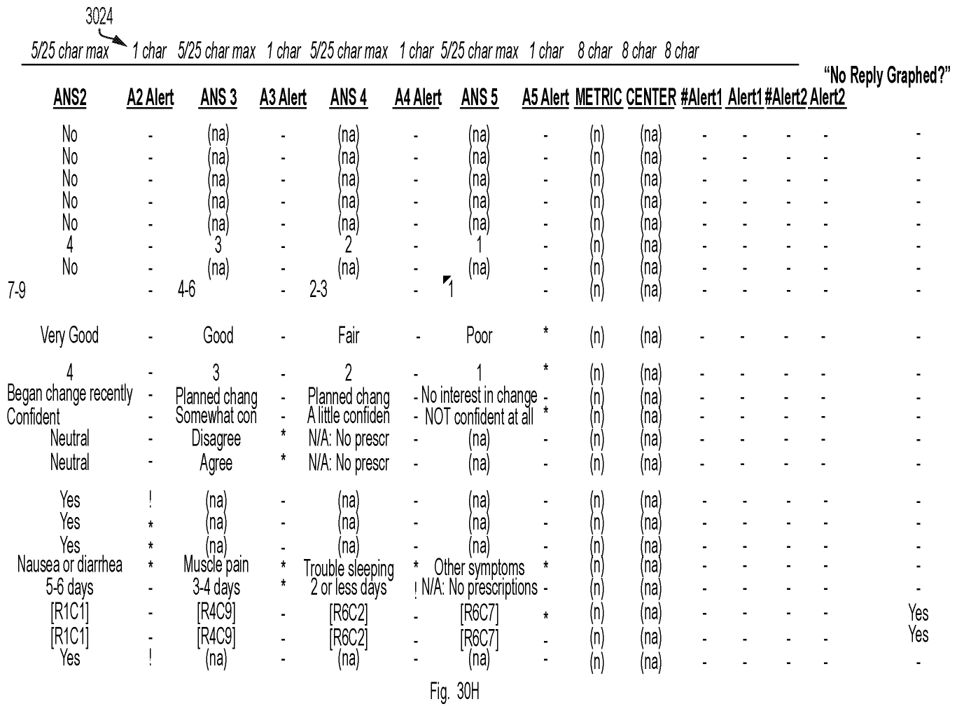

FIG. 30A depicts an example journey 3010 involving medications. Here, the journey data such as question, answers, alerts, etc. may be displayed on a user interface. In this example, the journey assesses patient understanding of the processes, patient attitudes regarding their health and medications, any changes to social determinants of health, and routinely asks about transitions of care, medication adherence, mood, and sleep patterns. This journey first asks about how well the patient understands how to use the app and what types of technology already in use by the patient. It also asks questions related to how the patient feels about his health and home situation. These questions will be repeated every few months or after major life events because views may change over time. Questions are also asked weekly about things that are important for us to know, such as if the patient has been in the ER or if the patient would like to be contacted. The journey assesses basic patient understanding of the processes and procedures in addition to identifying comfort level with technology. The journey asks about how well the patient understands how to use the system and what types of technology are already in use by the patient. The example seeks to ensure that the patient is comfortable with how the system will send messages and who will review them. Example questions are shown in the journey 3012 of FIG. 30B 3012.

FIG. 30C depicts an assessment 3014 of the patient's self-reported health status, willingness to change, as well as attitude toward medication adherence. This journey asks questions related to how the patient feels about his health. These questions will be repeated every few months or after major life events because views on health may change over time.

FIG. 30D is an example assessment 3016 of the patient's social determinants of health, including ability to perform ADL's and recent life changes. This journey asks questions related to the patient's home situation to better understand other factors that could be affecting health.

FIG. 30E is an example journey assessment 3018 for patient events, such as ER visits, in-patient admissions, and new prescriptions or physician visits. It also addresses patient satisfaction and provides the option for the patient to be contacted by their physician. This journey asks questions related to events that would be important for the health care provider to know. For example, if a patient visits an ER that is not affiliated with the health care provider's office. There is also an option to be contacted by the health care provider if there are questions or concerns. FIG. 30F 3020 depicts example questions for the assessment of 3018.

FIGS. 30G and 30H are examples of journeys 3022 and 3024, respectively, which include medication question categories, tags, form-identification, question number, question, layout of the answers whether horizontal or vertical and answers and alerts associated with the patient answers and the graphing of the answers. The figures are similar to FIGS. 29I and 29J.

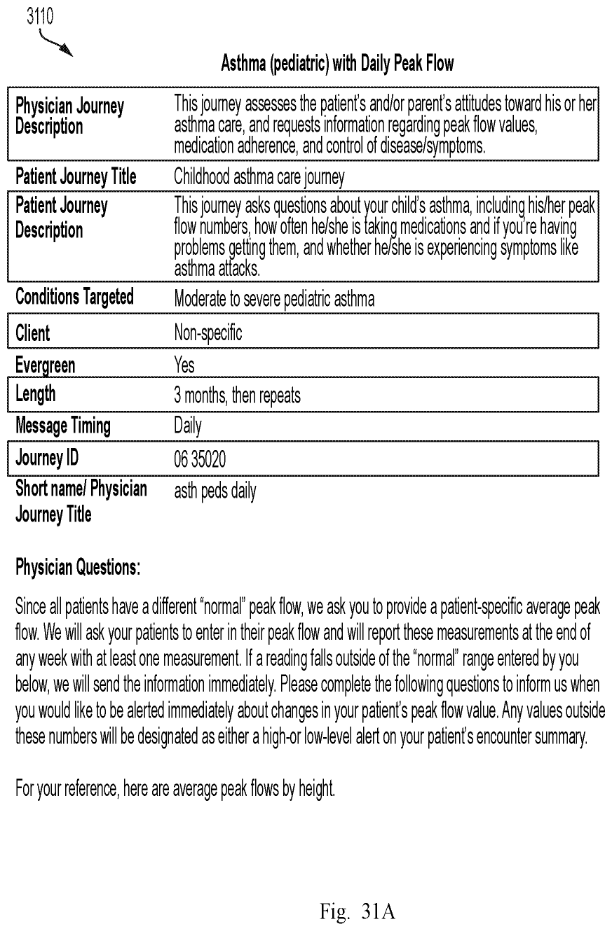

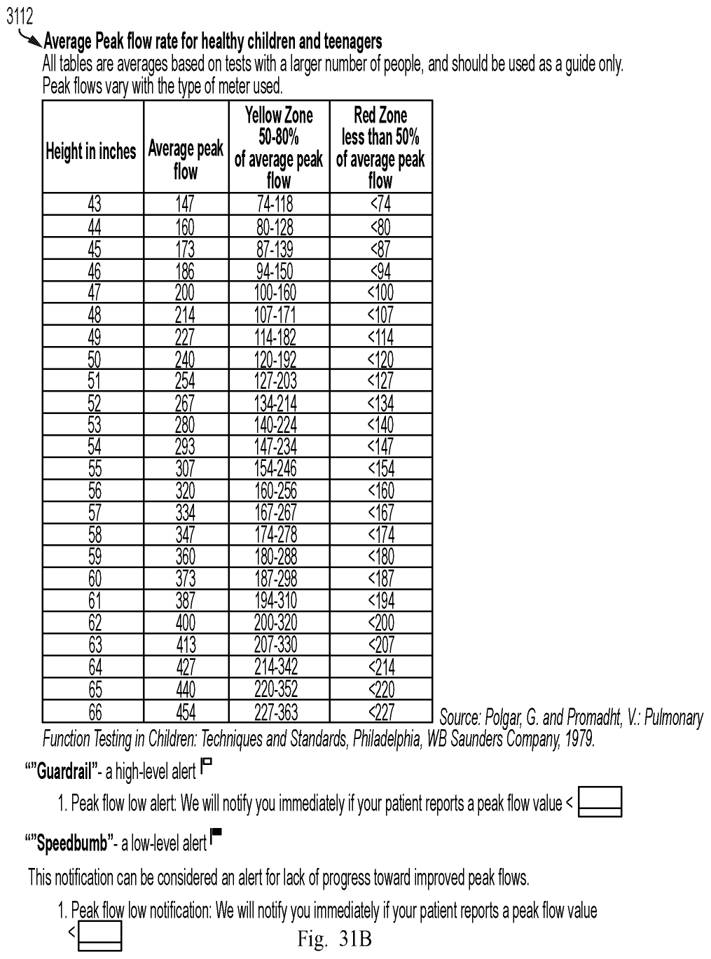

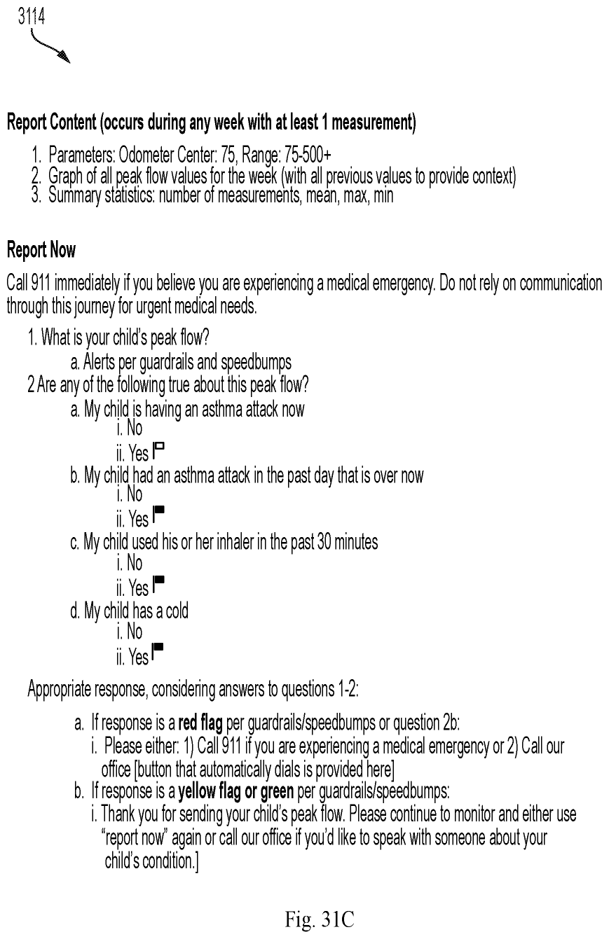

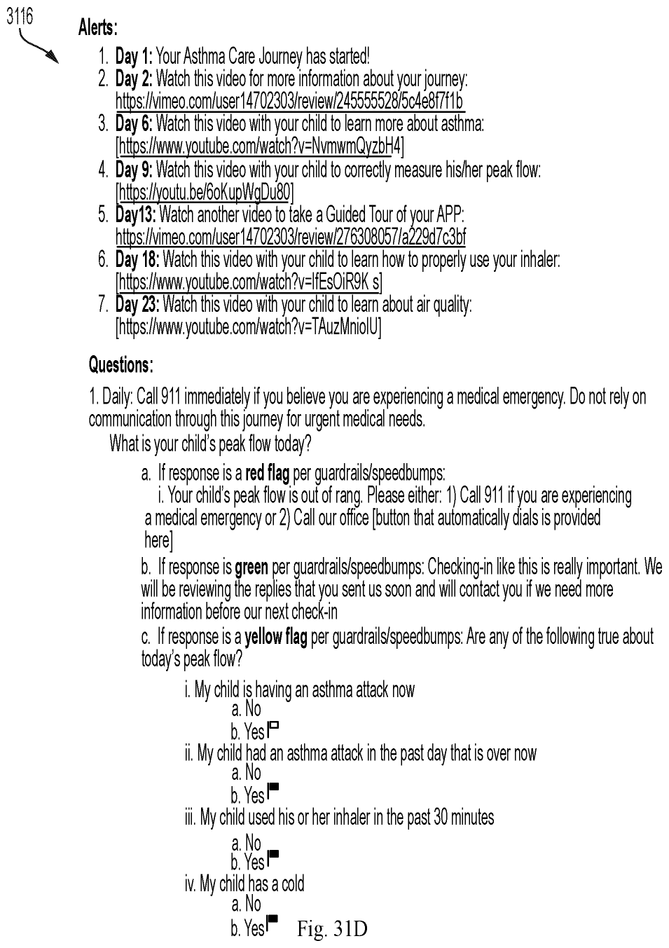

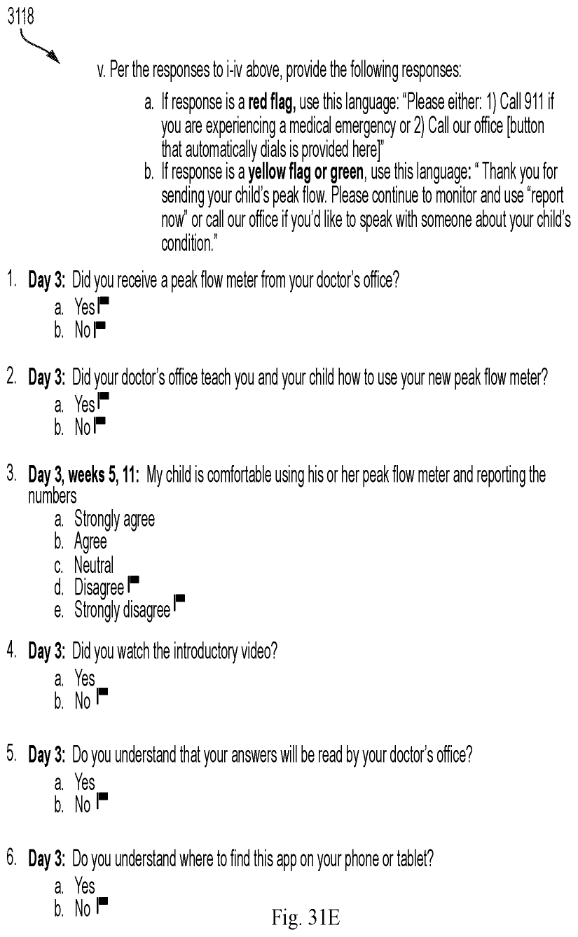

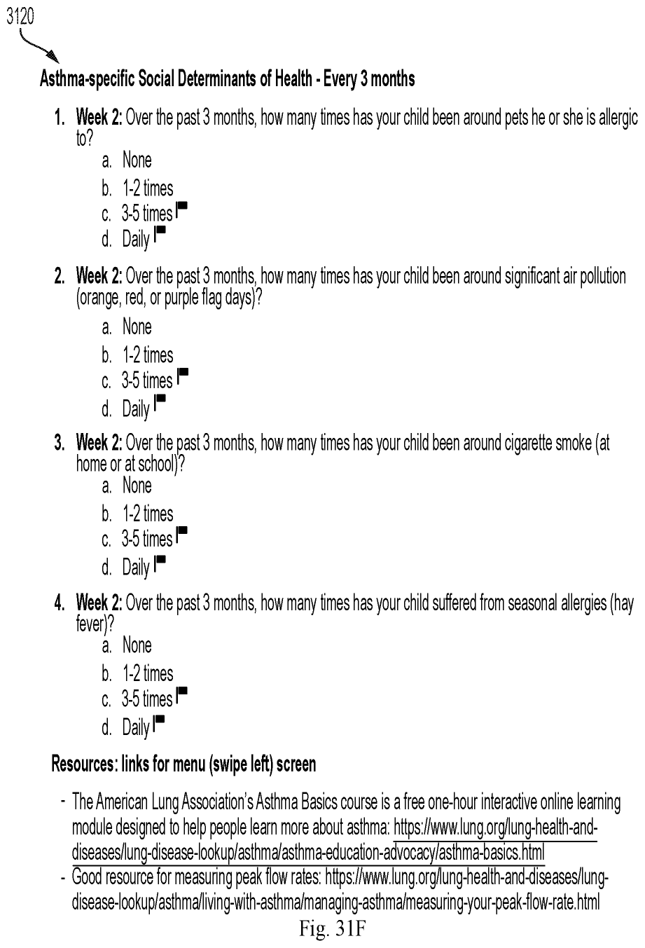

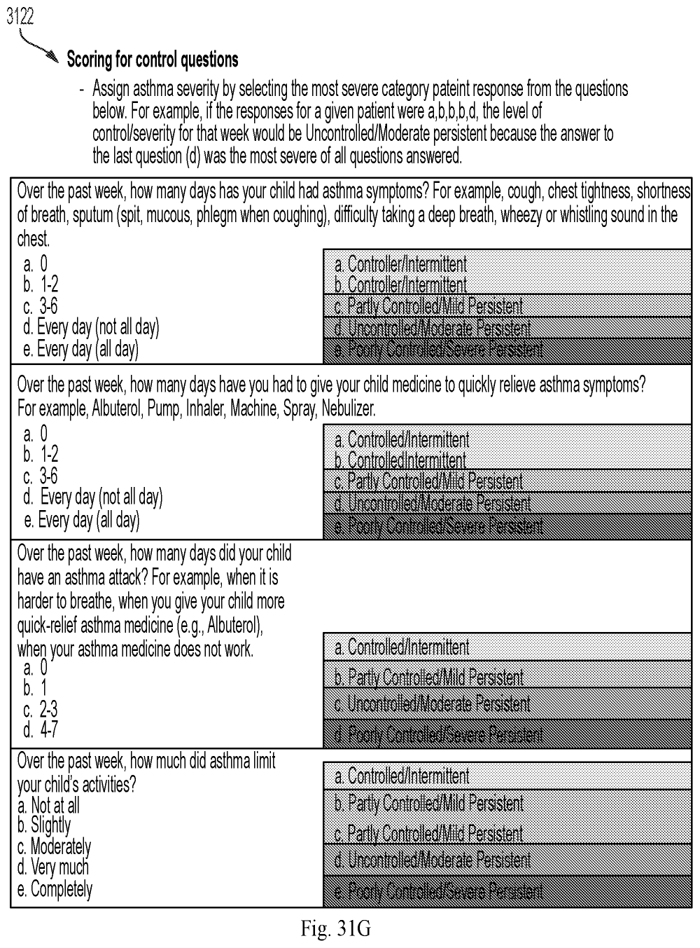

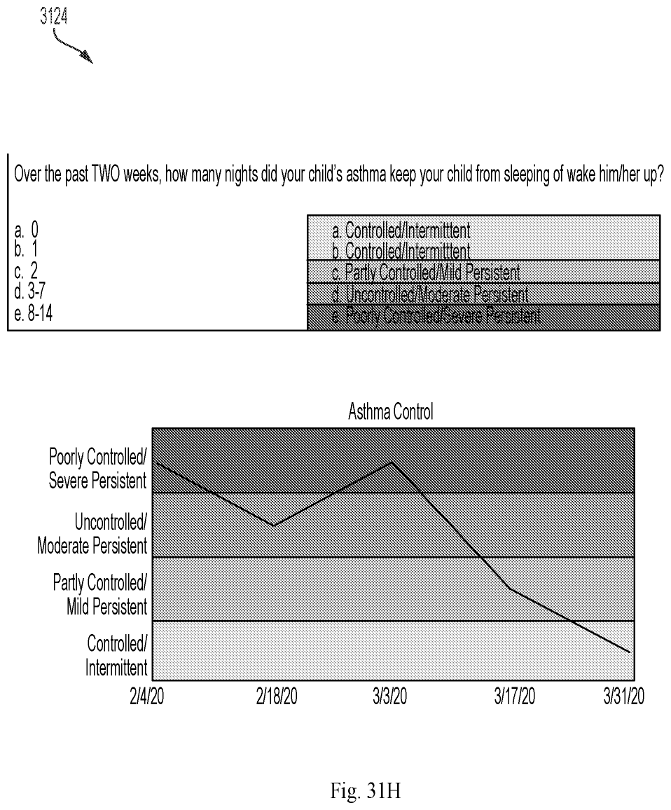

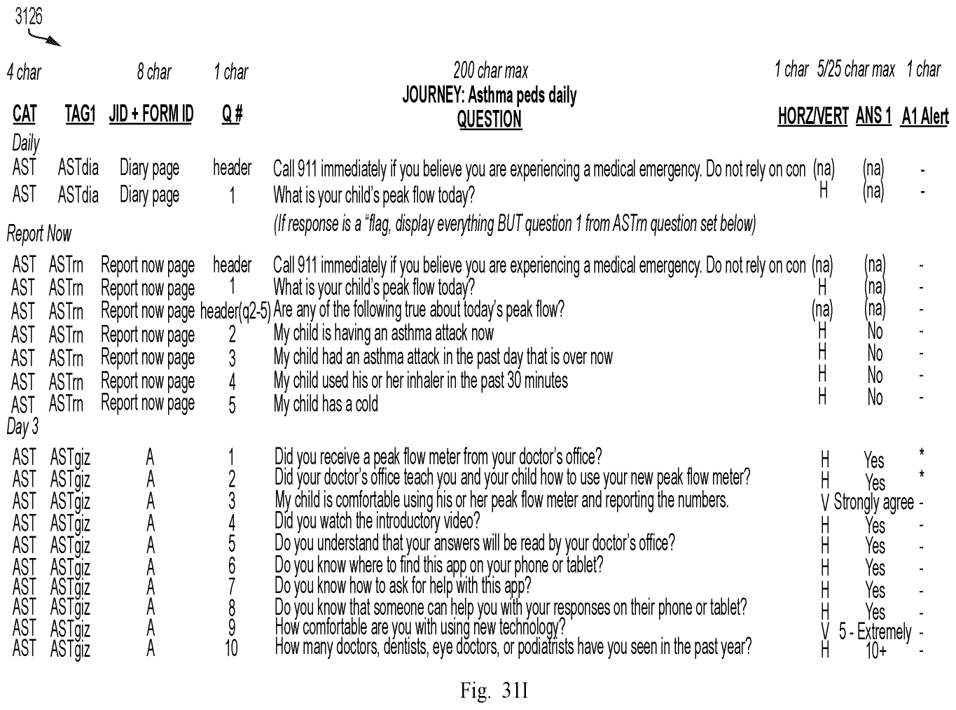

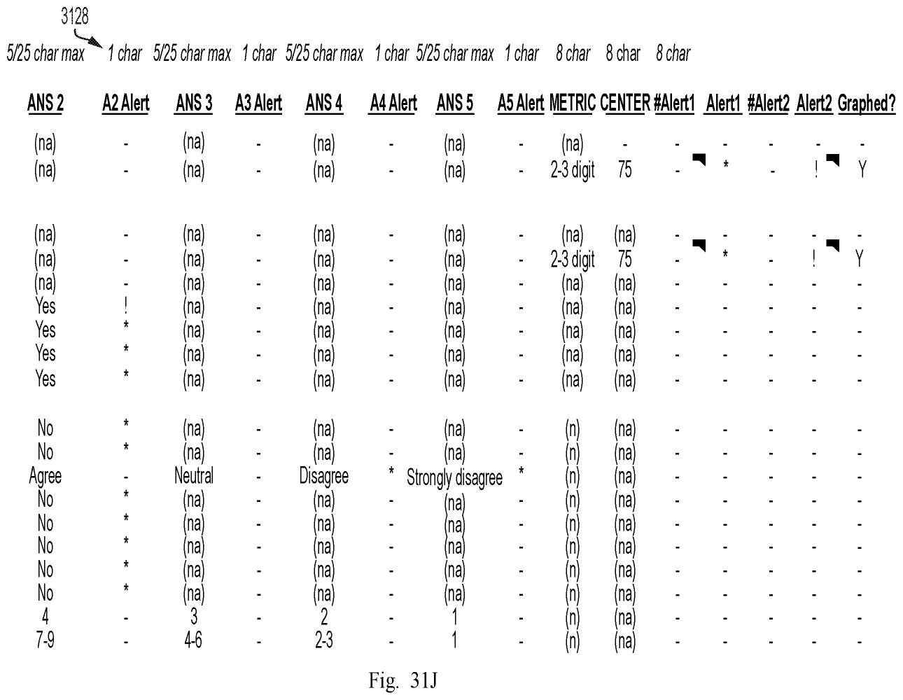

FIG. 31A depicts an example pediatric asthma journey 3110 displayed on a user interface of the application. This journey assesses the patient's and/or parent's attitudes toward his or her asthma care, and requests information regarding peak flow values, medication adherence, and control of disease/symptoms. This journey asks questions about the child's asthma, including his/her peak flow numbers, how often he/she is taking medications and if the child is having problems getting them, and whether he/she is experiencing symptoms like asthma attacks. FIG. 31B includes a user interface 3112 which shows peak flow rates for heathy children and teenagers, guardrails, and speedbumps. FIG. 31C includes a user interface 3114 that shows example report content and immediate reporting situations. FIG. 31D includes a user interface 3116, FIG. 31E includes a user interface 3118, and FIG. 31F includes a user interface 3120 which each show example alerts and questions and resources. FIG. 31G includes a user interface 3122 and FIG. 31H includes a user interface 3124 which show example scoring for control questions and asthma control chart.

FIG. 31I includes a user interface 3126 and FIG. 31J includes a user interface 3128 which show example pediatric asthma question categories, tags, form-identification, question number, question, layout of the answers whether horizontal or vertical and answers and alerts associated with the patient answers and the graphing of the answers. The figures are similar to FIGS. 29I and 29J.

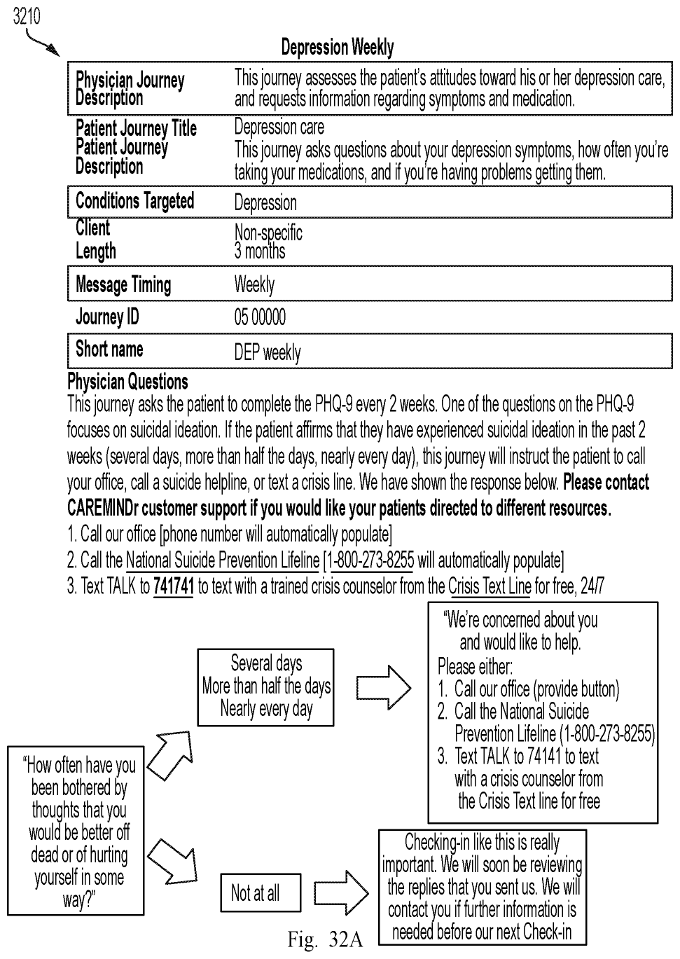

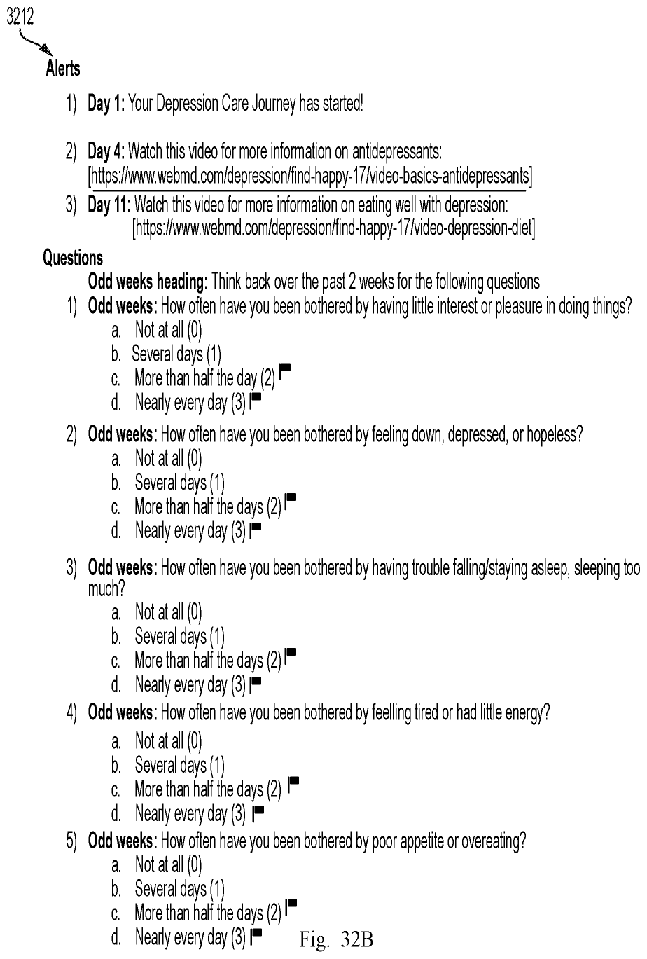

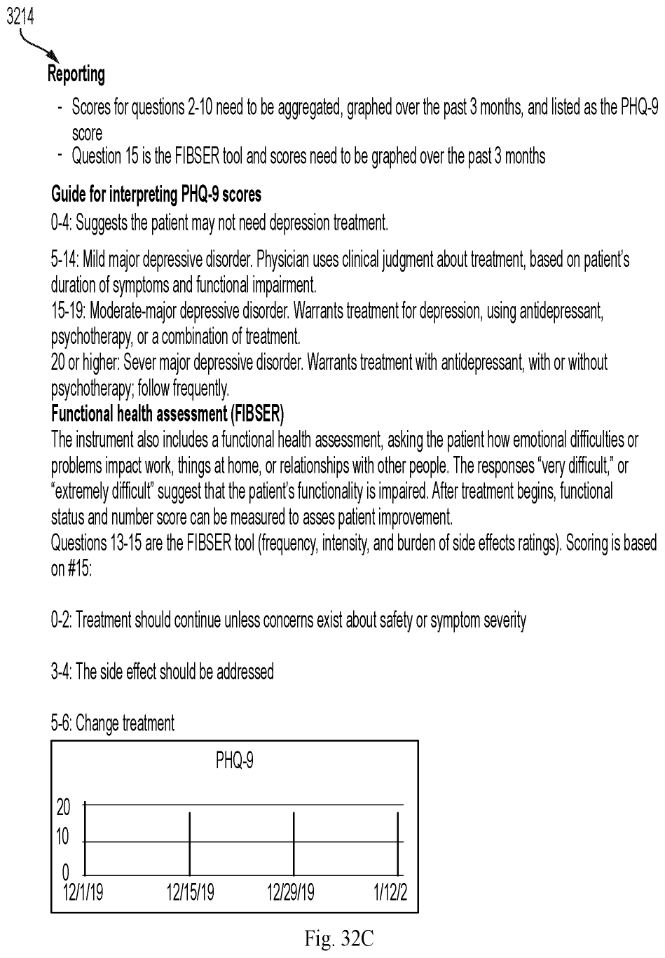

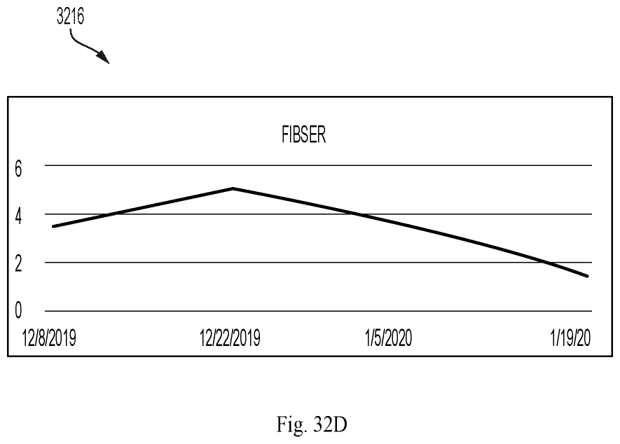

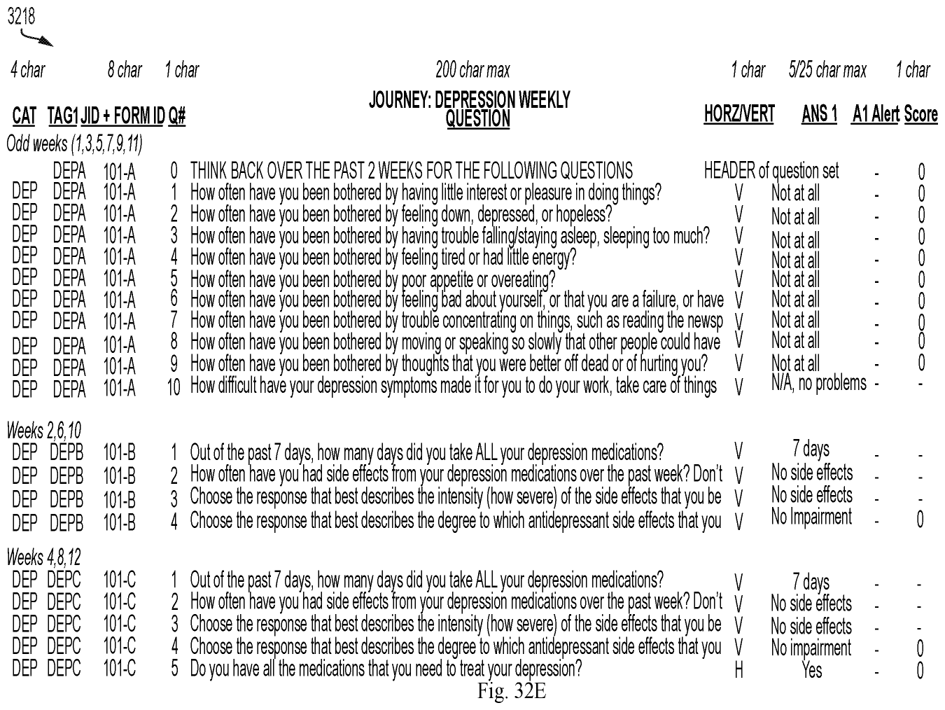

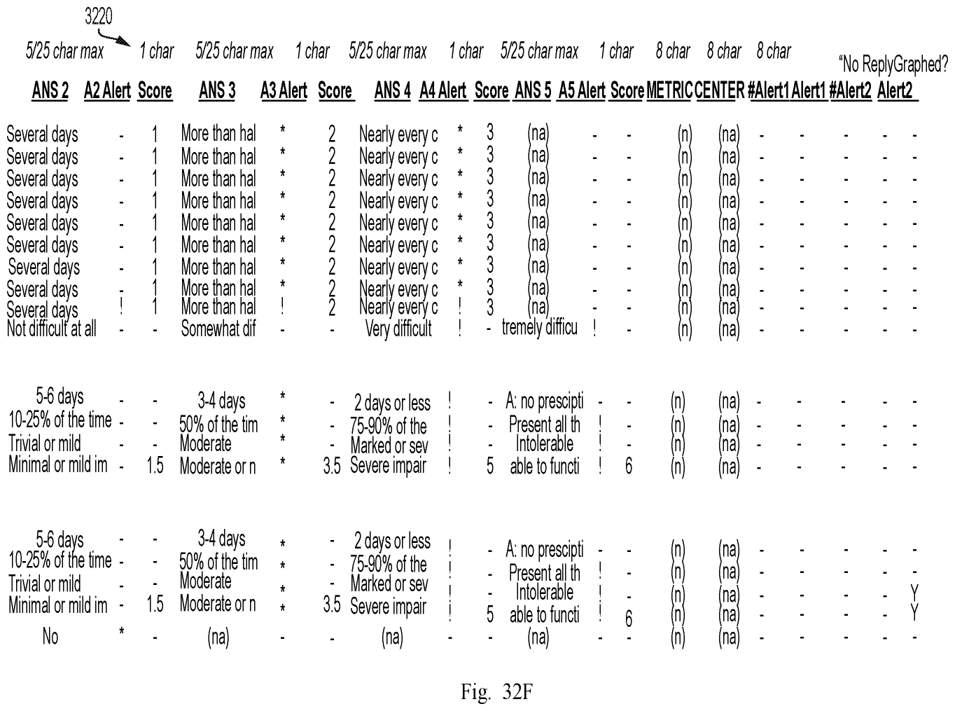

FIG. 32A depicts an example depression journey 3210. This journey assesses the patient's attitudes toward his or her depression care, and requests information regarding symptoms and medication. This journey asks questions about the patient's depression symptoms, how often the patient takes their medications, and if the patient is having problems getting them. FIG. 32B includes a user interface 3212 that depicts example depression alerts and questions. FIG. 32C includes a user interface 3214 that depicts example reporting, guides for PHQ-9 scores, functional assessments and output figures including the figures shown in user interface 3216 of FIG. 32D.

FIG. 32E includes a user interface 3218 and FIG. 32F includes a user interface 3220 which each include an example of depression journey question categories, tags, form-identification, question number, question, layout of the answers whether horizontal or vertical and answers and alerts associated with the patient answers and the graphing of the answers. The figures are similar to FIGS. 29I and 29J.

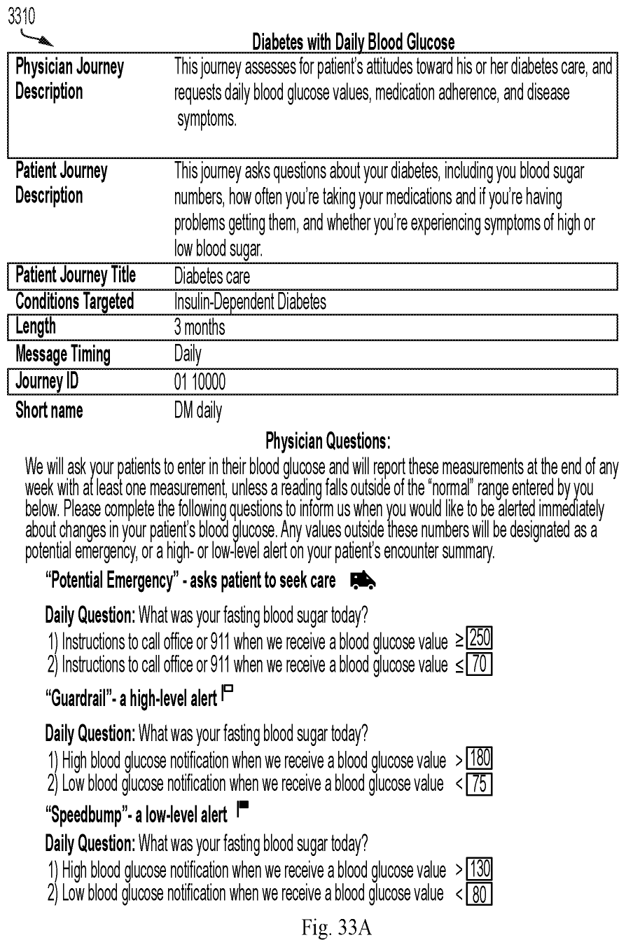

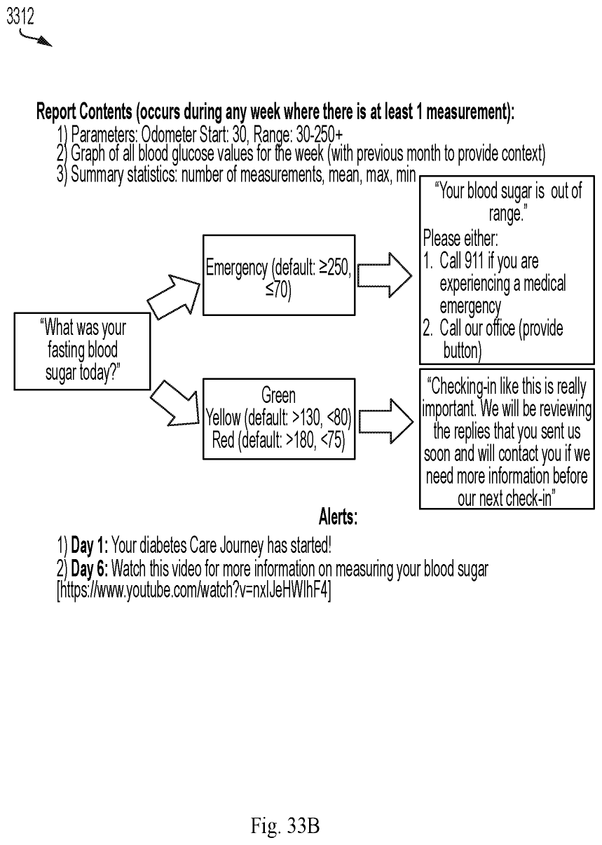

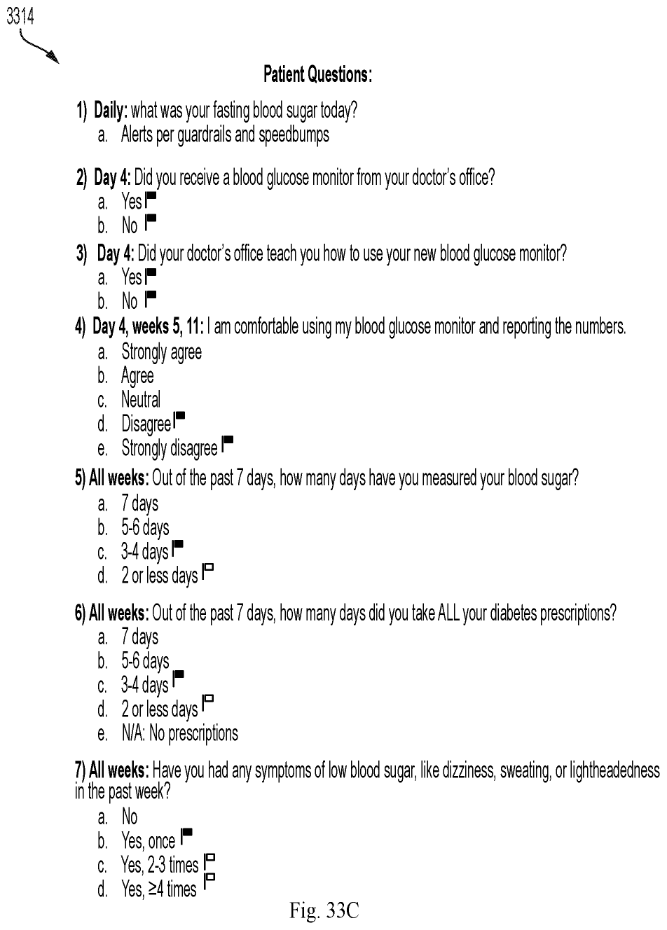

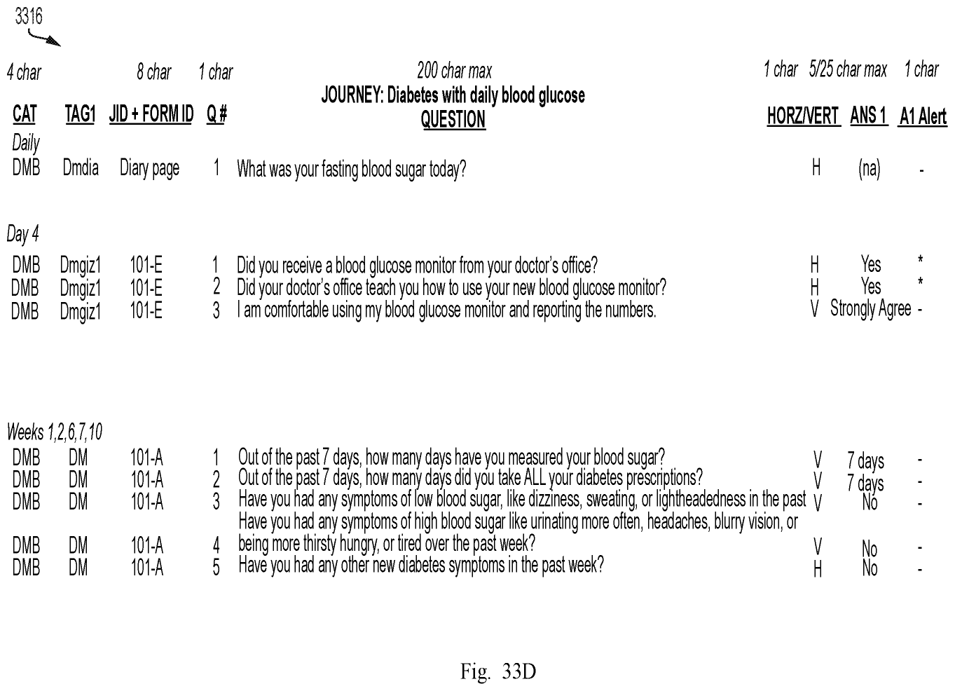

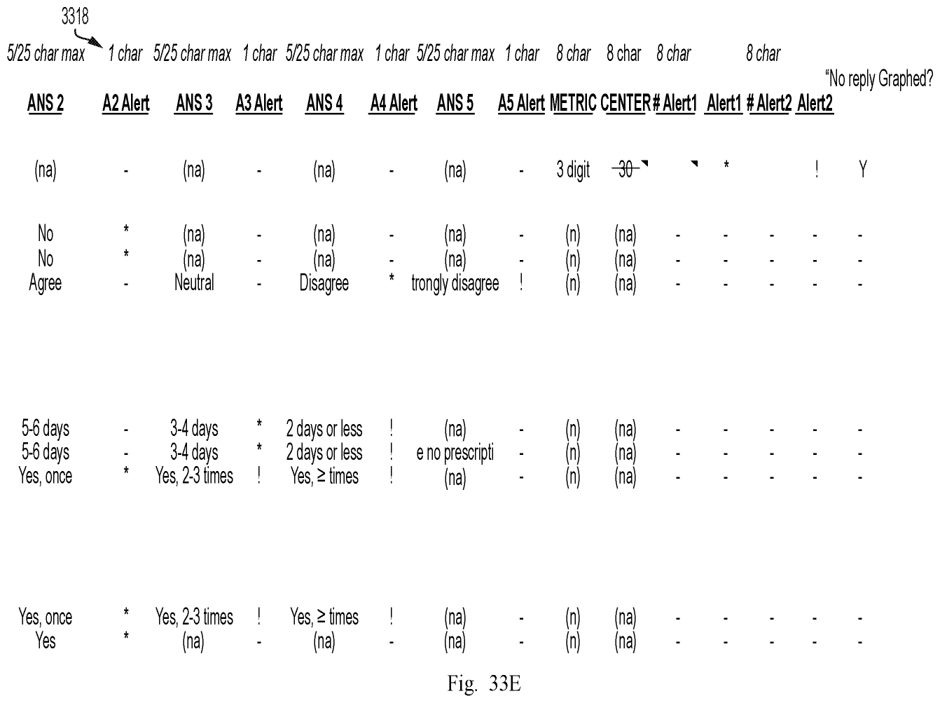

FIG. 33A depicts an example diabetes with daily blood glucose journey 3310. The diabetes journey assesses the patient's attitudes toward his or her diabetes care, and requests daily blood glucose values, medication adherence, and disease symptoms. This journey asks questions about the patient's diabetes, including blood sugar numbers, how often the patient takes his medications and if the patient is having problems getting them, and whether the patient is experiencing symptoms of high or low blood sugar. The example also indicates potential emergency situations, guardrails and speedbumps. FIG. 33B includes a user interface 3312 that depicts report contents. FIG. 33C 3314 depicts example questions.

FIG. 33D includes a user interface 3216 and FIG. 33E includes a user interface 3218 with example diabetes journey question categories, tags, form-identification, question number, question, layout of the answers whether horizontal or vertical and answers and alerts associated with the patient answers and the graphing of the answers.

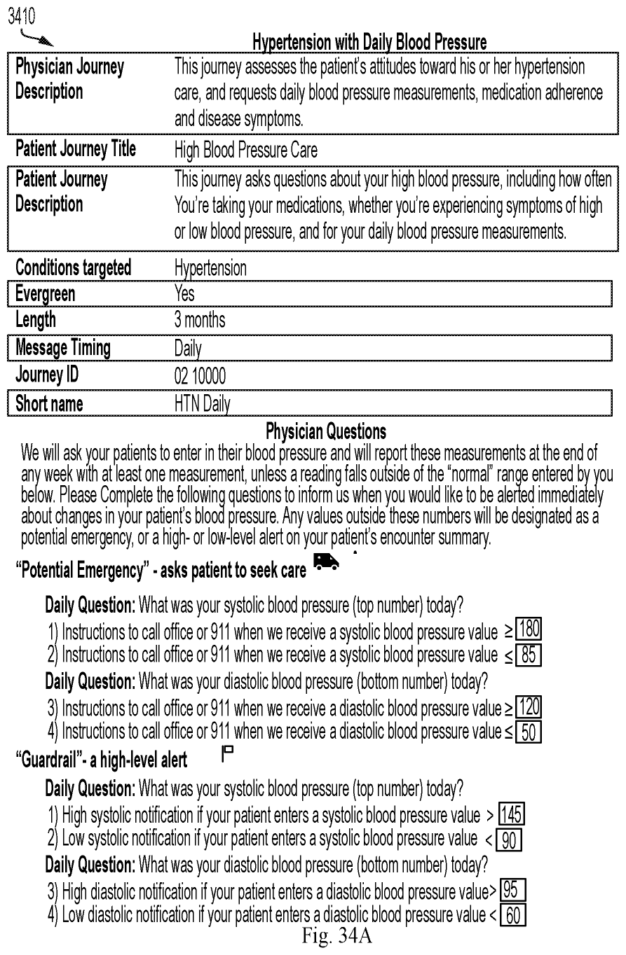

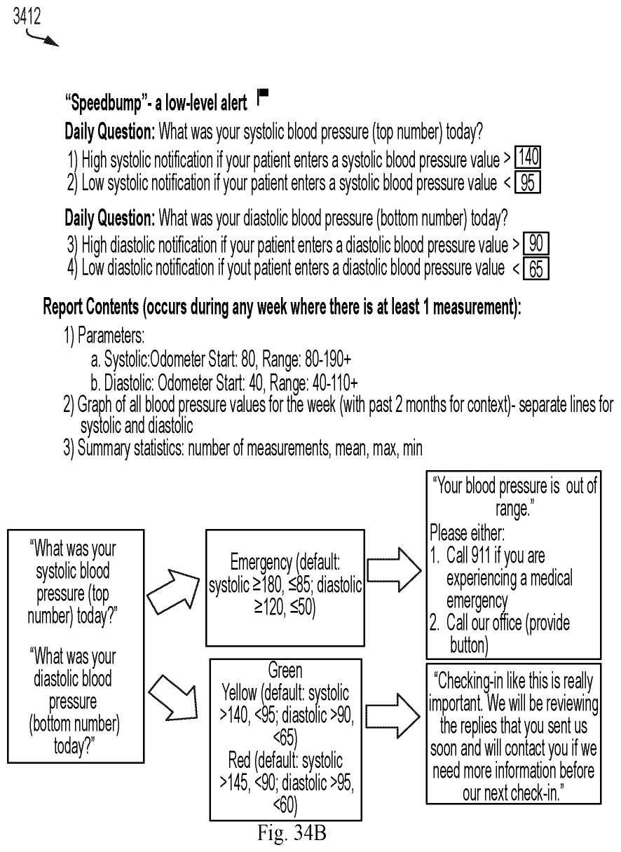

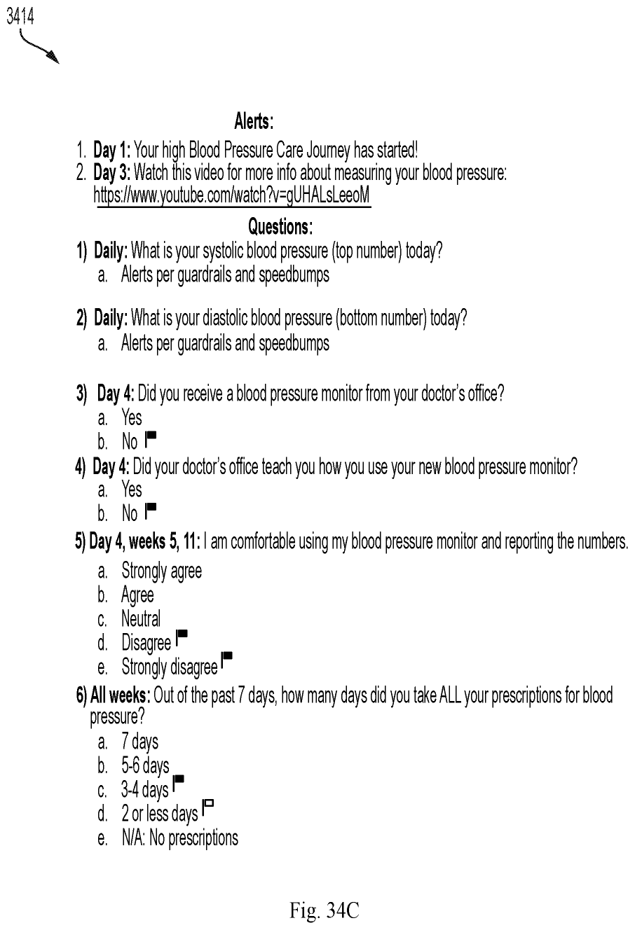

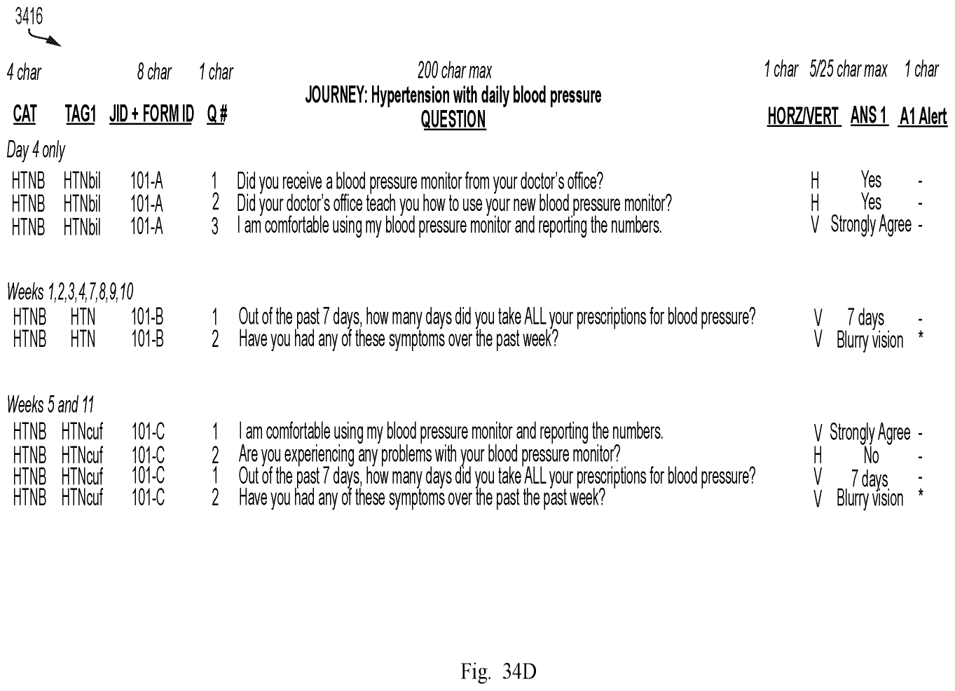

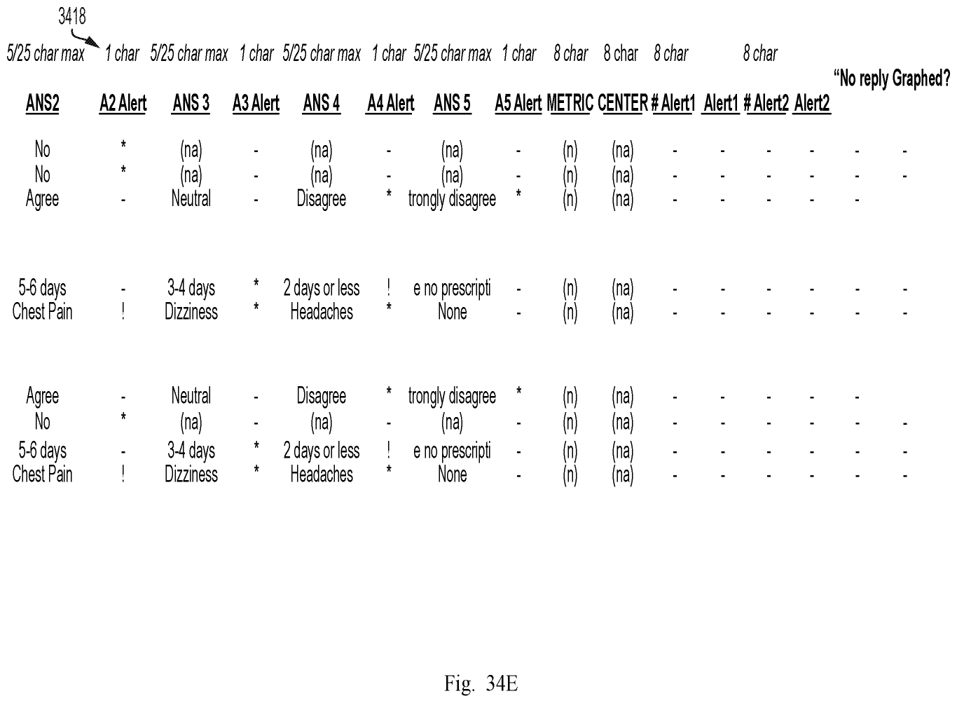

FIG. 34A depicts an example hypertension journey 3410. This journey assesses the patient's attitudes toward his or her hypertension care, and requests daily blood pressure measurements, medication adherence and disease symptoms. This journey asks questions about his or her high blood pressure, including how often the patient is taking medications, whether the patient is experiencing symptoms of high or low blood pressure, and for the patient's daily blood pressure measurements. FIG. 34A also depicts example potential emergencies and guardrails. FIG. 34B includes a user interface 3412 that depicts an example speedbump low level alert and report contents. FIG. 34C includes a user interface 3414 that depicts example alerts and questions.

FIG. 34D includes a user interface 3416 and FIG. 34E includes a user interface 3418 which are example hypertension journey question categories, tags, form-identification, question number, question, layout of the answers whether horizontal or vertical and answers and alerts associated with the patient answers and the graphing of the answers. The figures are similar to FIGS. 29I and 29J.

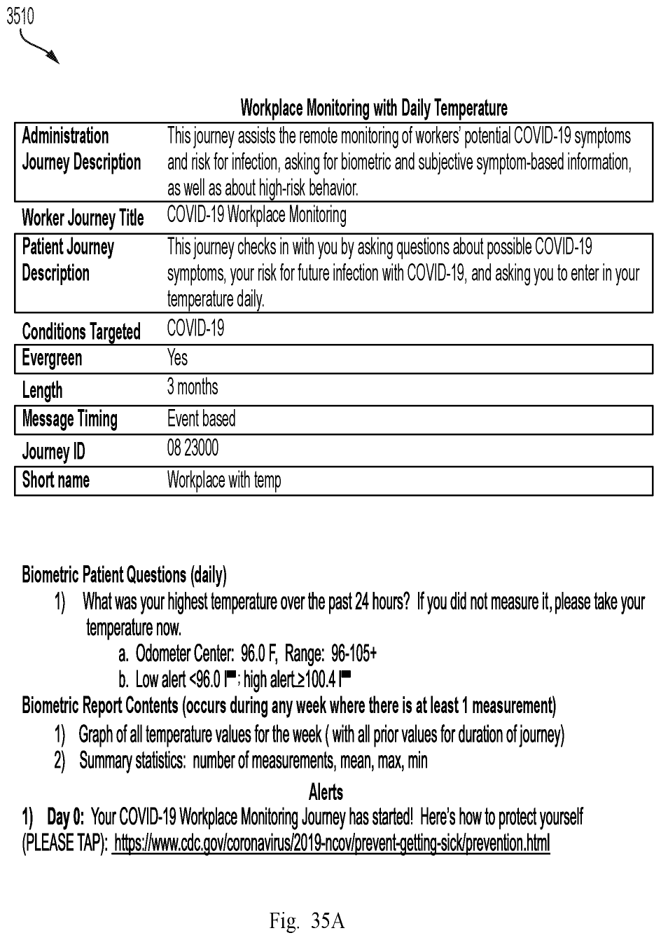

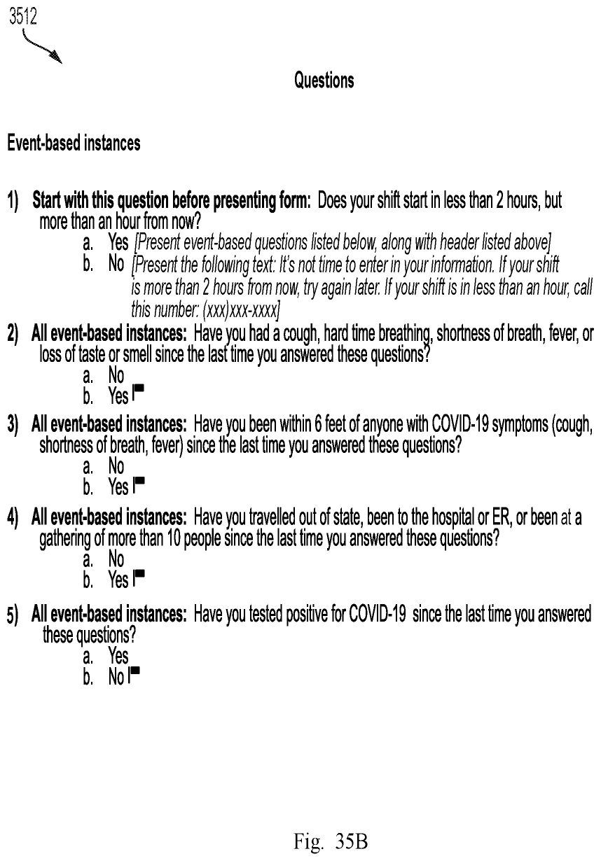

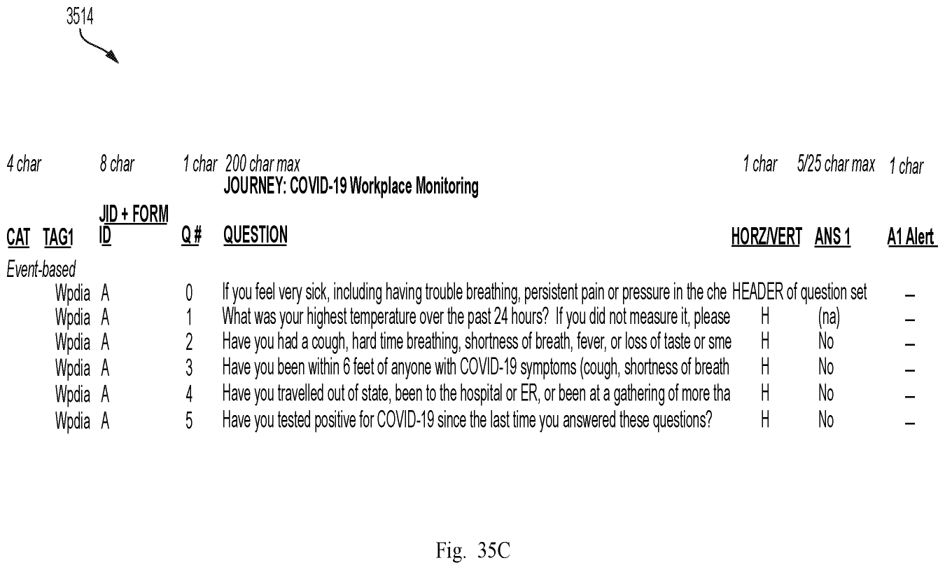

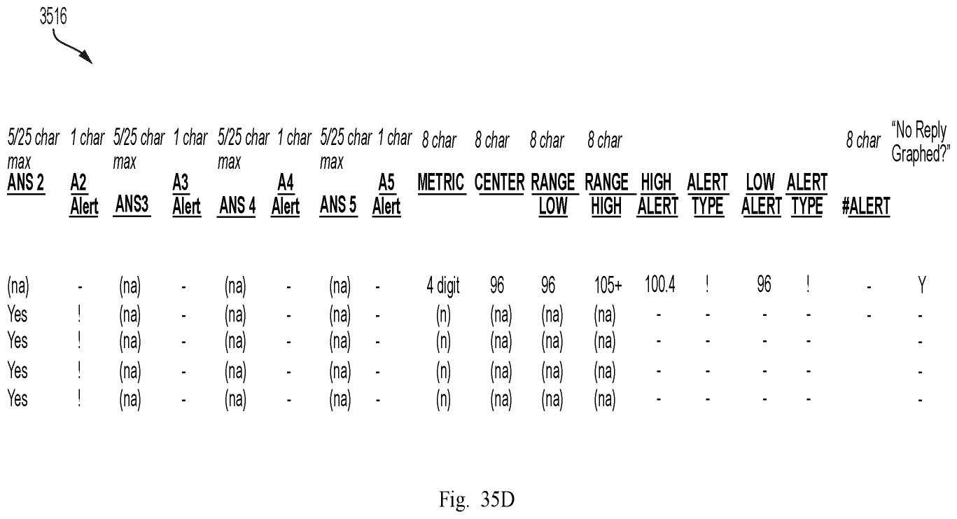

FIG. 35A depicts an example workplace monitoring journey 3510. This journey assists the remote monitoring of workers' potential COVID-19 symptoms and risk for infection, asking for biometric and subjective symptom-based information, as well as about high-risk behavior. This journey checks in with the patient by asking questions about possible COVID-19 symptoms, the patient's risk for future infection with COVID-19 and asking the patent to enter in their temperature daily. FIG. 35B depicts example questions.

FIG. 35C includes a user interface 3514 and FIG. 35D includes a user interface 3516 with example workplace monitoring journey question categories, tags, form-identification, question number, question, layout of the answers whether horizontal or vertical and answers and alerts associated with the patient answers and the graphing of the answers. The figures are similar to FIGS. 29I and 29J.

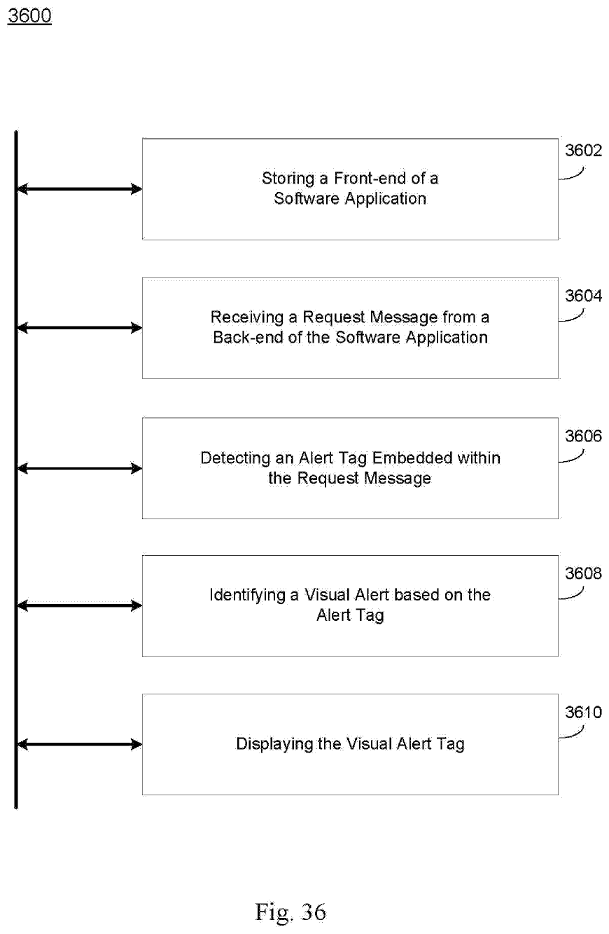

FIG. 36 illustrates a method 3600 of displaying an alert via a software application. Referring to FIG. 36, in 3602, the method may include storing a front-end of a software application. In 3604, the method may include receiving, from a host server, a request message from a back-end of the software application. In 3606, the method may include detecting an alert tag which is embedded within the request message by the host server. In 3608, the method may include identifying a visual alert based on the alert tag. In 3610, the method may include displaying data about the request message along with the visual alert via a user interface of the front-end of the software application.

In some embodiments, the request message may include a hypertext transfer protocol (HTTP) message, and the alert tag is embedded within a header of the HTTP message. In some embodiments, the identifying may include identifying the visual alert based on a mapping of the visual alert to the alert tag included in a mapping table. In some embodiments, the mapping table comprises a plurality of different alert tags corresponding to a plurality of levels of urgency, respectively, and each level of urgency is mapped to a different visual alert in the mapping table. In some embodiments, the detecting may include detecting an alert tag that indicates a lack of response from a user, and the displaying may include displaying a visual alert that indicates the lack of response from the user.

In some embodiments, the displaying may include displaying a status window and populating the status window with a plurality of bubbles corresponding to a plurality of users of the software application. In some embodiments, the displaying may include populating two status bubbles corresponding to two different users with two different visual alerts, and re-arranging the two status bubbles based on the two different visual alerts.

The operations of a method or algorithm described in connection with the embodiments disclosed herein may be embodied directly in hardware, in a computer program executed by a processor, or in a combination of the two. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

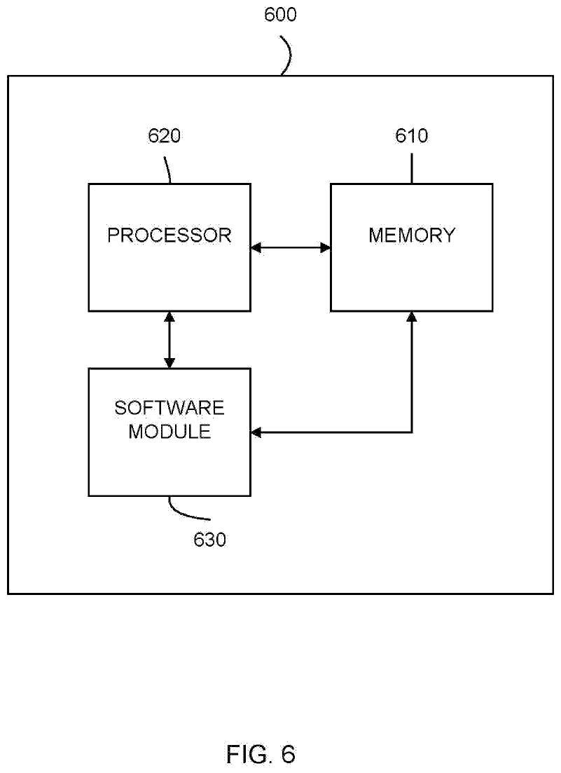

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 6 illustrates an example network element, which may represent any of the above-described network components of the other figures.

As illustrated in FIG. 6, a memory 610 and a processor 620 may be discrete components of the network entity 600 that are used to execute an application or set of operations. The application may be coded in software in a computer language understood by the processor 620, and stored in a computer readable medium, such as, the memory 610. The computer readable medium may be a non-transitory computer readable medium that includes tangible hardware components in addition to software stored in memory. Furthermore, a software module 630 may be another discrete entity that is part of the network entity 600, and which contains software instructions that may be executed by the processor 620. In addition to the above noted components of the network entity 600, the network entity 600 may also have a transmitter and receiver pair configured to receive and transmit communication signals (not shown).

Although an exemplary embodiment of the system, method, and computer readable medium of the present application has been illustrated in the accompanied drawings and described in the foregoing detailed description, it will be understood that the application is not limited to the embodiments disclosed, but is capable of numerous rearrangements, modifications, and substitutions without departing from the spirit or scope of the application as set forth and defined by the following claims. For example, the capabilities of the system of the various figures can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the present application in any way, but is intended to provide one example of many embodiments of the present application. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed but is merely representative of selected embodiments of the application.

One having ordinary skill in the art will readily understand that the application as discussed above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent, while remaining within the spirit and scope of the application. In order to determine the metes and bounds of the application, therefore, reference should be made to the appended claims.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

What is claimed is:

1. A computing system comprising:
a memory configured to store a front-end of a software application; and
a processor configured to
receive, from a host server, a request message from a back-end of the software application,
detect an alert tag which is embedded within the request message by the host server,
identify a visual alert based on the alert tag and display data about the request message along with the visual alert via a user interface of the front-end of the software application.

2. The computing system of claim 1, wherein the request message comprises a hypertext transfer protocol (HTTP) message, and the alert tag is embedded within a header of the HTTP message.

3. The computing system of claim 1, wherein the processor is configured to identify the visual alert based on a mapping of the visual alert to the alert tag included in a mapping table.

4. The computing system of claim 3, wherein the mapping table comprises a plurality of different alert tags corresponding to a plurality of levels of urgency, respectively, and each level of urgency is mapped to a different visual alert in the mapping table.

5. The computing system of claim 1, wherein the processor is configured to detect an alert tag that indicates a lack of response from a user, and display a visual alert that indicates the lack of response from the user.

6. The computing system of claim 1, wherein the processor is configured to display a status window and populate the status window with a plurality of bubbles corresponding to a plurality of users of the software application.

7. The computing system of claim 6, wherein the processor is configured to populate two status bubbles corresponding to two different users with two different visual alerts, and re-arrange the two status bubbles based on the two different visual alerts.

8. A method comprising:
storing a front-end of a software application;
receiving, from a host server, a request message from a back-end of the software application;
detecting an alert tag which is embedded within the request message by the host server;
identifying a visual alert based on the alert tag; and
displaying data about the request message along with the visual alert via a user interface of the front-end of the software application.

9. The method of claim 8, wherein the request message comprises a hypertext transfer protocol (HTTP) message, and the alert tag is embedded within a header of the HTTP message.

10. The method of claim 8, wherein the identifying comprises identifying the visual alert based on a mapping of the visual alert to the alert tag included in a mapping table.

11. The method of claim 10, wherein the mapping table comprises a plurality of different alert tags corresponding to a plurality of levels of urgency, respectively, and each level of urgency is mapped to a different visual alert in the mapping table.

12. The method of claim 8, wherein the detecting comprises detecting an alert tag that indicates a lack of response from a user, and the displaying comprises displaying a visual alert that indicates the lack of response from the user.

13. The method of claim 8, wherein the displaying comprises displaying a status window and populating the status window with a plurality of bubbles corresponding to a plurality of users of the software application.

14. The method of claim 13, wherein the displaying comprises populating two status bubbles corresponding to two different users with two different visual alerts, and re-arranging the two status bubbles based on the two different visual alerts.

15. A non-transitory computer-readable medium comprising instructions which when executed by a processor cause a computer to perform a method comprising:
storing a front-end of a software application;
receiving, from a host server, a request message from a back-end of the software application;
detecting an alert tag which is embedded within the request message by the host server;
identifying a visual alert based on the alert tag; and
displaying data about the request message along with the visual alert via a user interface of the front-end of the software application.

16. The computer-readable medium of claim 15, wherein the request message comprises a hypertext transfer protocol (HTTP) message, and the alert tag is embedded within a header of the HTTP message.

17. The computer-readable medium of claim 15, wherein the identifying comprises identifying the visual alert based on a mapping of the visual alert to the alert tag included in a mapping table.

18. The computer-readable medium of claim 17, wherein the mapping table comprises a plurality of different alert tags corresponding to a plurality of levels of urgency, respectively, and each level of urgency is mapped to a different visual alert in the mapping table.

19. The computer-readable medium of claim 15, wherein the displaying comprises displaying a status window and populating the status window with a plurality of bubbles corresponding to a plurality of users of the software application.

20. The computer-readable medium of claim 20, wherein the displaying comprises populating two status bubbles corresponding to two different users with two different visual alerts, and re-arranging the two status bubbles based on the two different visual alerts.
